(19) United States
(12) Patent Application Publication
(10) Pub. No.: US 2025/0295699 A1
(43) Pub. Date: Sep. 25, 2025

(54) METHODS AND COMPOSITIONS FOR TREATING CORTICAL HYPEREXCITABILITY AND REVERSING STROKE-INDUCED CHANGES IN GENE EXPRESSION AND PROTEIN EXPRESSION

(71) Applicant: SanBio, Inc., Oakland, CA (US)

(72) Inventors: Barbara KLEIN, Foster City, CA (US); Yaisa ANDREWS-ZWILLING, Havertown, PA (US); Zuha WARRAICH, San Francisco, CA (US); Jeanne T. PAZ, San Francisco, CA (US)

(73) Assignee: SanBio, Inc., Oakland, CA (US)

(21) Appl. No.: 19/084,488

(22) Filed: Mar. 19, 2025

Related U.S. Application Data
(60) Provisional application No. 63/568,832, filed on Mar. 22, 2024.

(51) Int. Cl. A61K 35/28; A61K 9/00; A61K 9/08; A61P 9/10; C12N 5/0775

(52) U.S. Cl. CPC A61K 35/28; A61K 9/0019; A61K 9/0085; A61K 9/08; A61P 9/10; C12N 5/0663; C12N 2501/42

(57) ABSTRACT

Disclosed are compositions and methods for treating cortical hyperexcitability and reversing stroke-induced changes in gene expression and protein expression. For example, the methods can comprise administering vandefitemcel to a region of the brain of a subject to reduce chronic cortical hyperexcitability in the subject. Also disclosed are methods and blood biomarker panels for assessing an efficacy of a stem cell treatment for a chronic condition caused by stroke.

### CROSS-REFERENCE TO RELATED APPLICATION

This application claims the benefit of U.S. Provisional Application No. 63/568,832 filed on Mar. 22, 2024, the content of which is incorporated herein by reference in its entirety.

### TECHNICAL FIELD

The present disclosure relates generally to the field of cell therapies and, more specifically, to methods and compositions for treating cortical hyperexcitability and reversing stroke-induced changes in gene expression and protein expression.

### BACKGROUND

Despite increased knowledge of the pathological mechanisms triggered by stroke, the development of acute thrombolytic therapy, and subsequent neurorehabilitation training, ischemic stroke remains a common cause of adult neurological disability (Katan and Luft, 2018; Pu et al, 2023). Only about 5% of people who survive an ischemic stroke are fully cured, and the rest suffer from mild to severe, long-term to lifelong disability including motor disorders, chronic pain, sleep disruption and epilepsy (Paolucci et al., 2016; Dhamoon et al., 2017; Hasan et al., 2021; Mishra et al., 2022; Paz et al., 2013; Paz and Huguenard, 2015).

While the brain compensates for the lost functions, some aspects of post-stroke plasticity can be maladaptive. One of the main illustrations of this adverse plasticity is the development of pathological cortical hyperexcitability (Medalla et al., 2020; Paz et al., 2013; Mishra et al., 2022), which is associated with the development of motor spasticity (Li, 2017) and post-stroke epilepsy (Mishra et al., 2022; Paz et al., 2013; Paz and Huguenard, 2015; Olsen et al., 1987; Galovic et al., 2018; Xu, 2019; Galovic et al., 2021). These disabilities develop as a consequence of ischemic injury and accrue over time, even years after stroke (Dhamoon et al., 2017). In the weeks and months following ischemic injury, the ability of the peri-infarct zone to recover function gradually decreases and recovery can reach a plateau (Grefkes and Fink, 2020). The chronic cortical hyperexcitability induced by stroke is not well characterized and no treatment is available yet to prevent it.

Over the past several decades, mesenchymal stem cell-based (MSC-based) therapies have emerged as a new strategy for treating neurological disorders and injuries (He et al., 2020; Volkman and Offen, 2017). MSCs have low immunogenicity and can be isolated from different adult and birth tissues and cultured at great expansion capacity (Berebichez-Fridman and Montero-Olvera, 2018).

Vandefitemcel, also known as SB623 cells, are a type of human bone marrow-derived MSC cells. Recent studies have shown that intracerebral implantation of vandefitemcel or SB623 cells was safe and could improve a patient's motor functions (Kawabori, Masahito, et al., 2021; Steinberg, Gary K., et al., 2018). However, little is known about the effect of MSCs on chronic cortical hyperexcitability.

Therefore, there is a need for safe and effective therapies for ameliorating the detrimental effects of chronic cortical hyperexcitability. Such therapies should also have a beneficial effect on a patient's neural plasticity. Such therapies should also be shown to be effective in animal models for stroke.

### SUMMARY

Disclosed are methods and compositions for treating cortical hyperexcitability and reversing stroke-induced changes in gene expression and protein expression. Also disclosed is a method of assessing an efficacy of a treatment for a chronic condition caused by ischemic stroke.

In some aspects, a method of treating chronic cortical hyperexcitability is disclosed. The method comprising: administering vandefitemcel to a region of the brain of a subject to reduce chronic cortical hyperexcitability in the subject, wherein the vandefitemcel are cells descended from mesenchymal stem cells (MSCs) transiently-transfected by a polynucleotide encoding a Notch intracellular domain (NICD), wherein the vandefitemcel treats the chronic cortical hyperexcitability by at least one of: increasing production of gamma-aminobutyric acid (GABA) transporters 1 (GAT1) in the brain of the subject; and increasing production of brain-derived neurotrophic factors (BDNF) in the brain of the subject.

In some aspects, the vandefitemcel treats the chronic cortical hyperexcitability by increasing the production of GAT1 in a peri-stroke cortex of the brain of the subject.

In some aspects, the vandefitemcel treats the chronic cortical hyperexcitability by increasing the production of BDNF in a peri-stroke cortex of the brain of the subject.

In some aspects, the vandefitemcel treats the chronic cortical hyperexcitability by increasing the production of BDNF in a corpus callosum of the brain of the subject.

In some aspects, the vandefitemcel treats the chronic cortical hyperexcitability by increasing the production of BDNF in a somatosensory thalamus of the brain of the subject.

In some aspects, the vandefitemcel treats the chronic cortical hyperexcitability by increasing the production of BDNF in an internal capsule of the brain of the subject.

In some aspects, the vandefitemcel treats the chronic cortical hyperexcitability by increasing the production of BDNF in a contralesional hemisphere of the brain of the subject.

In some aspects, the vandefitemcel treats the chronic cortical hyperexcitability by increasing the production of BDNF in an ipsilesional hemisphere of the brain of the subject.

In some aspects, the vandefitemcel treats the chronic cortical hyperexcitability by increasing production of doublecortin-positive (DCX⁺) neuronal progenitor cells (NPCs) in the brain of the subject.

In some aspects, the vandefitemcel treats the chronic cortical hyperexcitability by increasing production of oligodendrocyte precursor cells (OPCs).

In some aspects, the OPCs are oligodendrocyte transcription factor 2-positive (Olig2⁺) OPCs.

In some aspects, the OPCs are proliferating cell nuclear antigen-positive (PCNA⁺) OPCs.

In some aspects, the vandefitemcel treats the chronic cortical hyperexcitability by increasing production of myelin basic protein (MBP) in a contralesional cortex of the subject.

In some aspects, the vandefitemcel treats the chronic cortical hyperexcitability by increasing production of glial fibrillary acidic protein-positive (GFAP⁺) astrocytes in the brain of the subject.

In some aspects, the vandefitemcel treats the chronic cortical hyperexcitability by increasing production of ionized calcium-binding adaptor molecule 1-positive (Iba1⁺) microglia in the brain of the subject.

In some aspects, the vandefitemcel treats the chronic cortical hyperexcitability by inducing synaptogenesis in a peri-stroke cortex of the brain of the subject.

In some aspects, the vandefitemcel treats the chronic cortical hyperexcitability by reducing glutamatergic synaptic vesicles in an ipsilesional region of a peri-stroke cortex of the brain compared to a contralesional region of the peri-stroke cortex.

In some aspects, the vandefitemcel treats the chronic cortical hyperexcitability by reducing perineuronal nets in an ipsilesional region of a peri-stroke cortex of the brain compared to a contralesional region of the peri-stroke cortex.

In some aspects, administering the vandefitemcel further includes administering the vandefitemcel by intracerebral implantation.

In some aspects, administering the vandefitemcel further includes injecting the vandefitemcel at multiple sites within the region of the brain.

In some aspects, administering the vandefitemcel further includes administering the vandefitemcel stereotactically via a burr hole in a skull of the subject.

In some aspects, the region of the brain is a peri-stroke cortex.

In some aspects, administering the vandefitemcel further includes administering the vandefitemcel by parenteral administration.

In some aspects, the vandefitemcel is suspended in a sterile isotonic crystalloid solution.

In some aspects, administering the vandefitemcel further includes administering between about 1.0 million cells and about 10.0 million cells.

In some aspects, the vandefitemcel is made by a method including: providing a culture of MSCs; contacting the culture of MSCs with the polynucleotide encoding the NICD, wherein the polynucleotide does not encode a full-length Notch protein, selecting cells that include the polynucleotide; and further culturing the selected cells in the absence of selection for the polynucleotide.

In some aspects, the MSCs are human bone marrow-derived cells.

In some aspects, a method of reversing stroke-induced changes in whole-blood gene expression is disclosed. The method comprising: administering vandefitemcel to a region of the brain of a subject, wherein the vandefitemcel are cells descended from mesenchymal stem cells (MSCs) transiently-transfected by a polynucleotide encoding a Notch intracellular domain (NICD), wherein the vandefitemcel reverses stroke-induced changes in whole-blood gene expression by at least one of: downregulating CD8a gene expression; downregulating CD8b gene expression; downregulating Uchl1 gene expression; downregulating Casp3 gene expression; downregulating Ube2s gene expression; downregulating Slc18a2 gene expression; and upregulating Fcgr2b gene expression.

In some aspects, the vandefitemcel reverses stroke-induced changes in whole-blood gene expression by downregulating Scarf1 gene expression.

In some aspects, the vandefitemcel reverses stroke-induced changes in whole-blood gene expression by downregulating Dusp1 gene expression.

In some aspects, the vandefitemcel reverses stroke-induced changes in whole-blood gene expression by downregulating Csnk2a1 gene expression.

In some aspects, administering the vandefitemcel further includes administering the vandefitemcel by intracerebral implantation.

In some aspects, administering the vandefitemcel further includes injecting the vandefitemcel at multiple sites within the region of the brain.

In some aspects, administering the vandefitemcel further includes administering the vandefitemcel stereotactically via a burr hole in a skull of the subject.

In some aspects, the region of the brain is a peri-stroke cortex.

In some aspects, administering the vandefitemcel further includes administering between about 1.0 million cells and 10.0 million cells.

In some aspects, the vandefitemcel is made by a method including: providing a culture of MSCs; contacting the culture of MSCs with the polynucleotide encoding the NICD, wherein the polynucleotide does not encode a full-length Notch protein, selecting cells that include the polynucleotide; and further culturing the selected cells in the absence of selection for the polynucleotide.

In some aspects, the MSCs are human bone marrow-derived cells.

In some aspects, the vandefitemcel is suspended in a sterile isotonic crystalloid solution.

In some aspects, the changes are induced by an ischemic stroke and wherein the vandefitemcel are administered thirty days or more after the ischemic stroke.

In some aspects, a method of reversing stroke-induced changes in serum protein expression is disclosed. The method comprising: administering vandefitemcel to a region of the brain of a subject, wherein the vandefitemcel are cells descended from mesenchymal stem cells (MSCs) transiently-transfected by a polynucleotide encoding a Notch intracellular domain (NICD), wherein the vandefitemcel reverses stroke-induced changes in serum protein expression by at least one of: upregulating Filamin A protein expression; downregulating Cathepsin L protein expression; downregulating ApoE protein expression; downregulating ARHGAP1 protein expression; and downregulating TALDO1 protein expression.

In some aspects, administering the vandefitemcel further includes administering the vandefitemcel by intracerebral implantation.

In some aspects, administering the vandefitemcel further includes injecting the vandefitemcel at multiple sites within the region of the brain.

In some aspects, administering the vandefitemcel further includes administering the vandefitemcel stereotactically via a burr hole in a skull of the subject.

In some aspects, the region of the brain is a peri-stroke cortex.

In some aspects, administering the vandefitemcel further includes administering between about 1.0 million cells and 10.0 million cells.

In some aspects, the vandefitemcel is made by a method comprising: providing a culture of MSCs; contacting the culture of MSCs with the polynucleotide encoding the NICD, wherein the polynucleotide does not encode a full-length Notch protein, selecting cells that include the polynucleotide; and further culturing the selected cells in the absence of selection for the polynucleotide.

In some aspects, the MSCs are human bone marrow-derived cells.

In some aspects, the vandefitemcel is suspended in a sterile isotonic crystalloid solution.

In some aspects, the changes are induced by an ischemic stroke and wherein the vandefitemcel are administered thirty days or more after the ischemic stroke.

In some aspects, a method of assessing an efficacy of a treatment for a chronic condition caused by stroke is disclosed. The method comprising: obtaining a first sample of blood from a subject; administering vandefitemcel to a region of the brain of a subject, wherein the vandefitemcel are cells descended from mesenchymal stem cells (MSCs) transiently-transfected by a polynucleotide encoding a Notch intracellular domain (NICD); obtaining a second sample of blood from a subject at least three days (or at least seven days) after administering the vandefitemcel; comparing gene expression levels between the second sample of blood and the first sample of blood for at least one of the following biomarker genes: CD8a, CD8b, Uchl1, Casp3, Ube2s, and Slc18a2; and classifying the vandefitemcel administration as effective in ameliorating the chronic condition caused by the stroke when at least one of the CD8a gene, the CD8b gene, the Uchl1 gene, the Casp3 gene, the Ube2s gene, and the Slc18a2 gene in the second sample of blood is downregulated relative to the first sample of blood.

In some aspects, the method further comprises: comparing a gene expression level between the second sample of blood and the first sample of blood for the biomarker gene Fcgr2b; and classifying the vandefitemcel administration as effective in ameliorating the chronic condition caused by the stroke when the biomarker gene Fcgr2b in the second sample of blood is upregulated relative to the first sample of blood.

In some aspects, the method further comprises: comparing gene expression levels between the second sample of blood and the first sample of blood for at least one of the following biomarker genes: Scarf1, Dusp1, and Csnk2a1; and classifying the vandefitemcel administration as effective in ameliorating the chronic condition caused by the stroke when at least one of the Scarf1 gene, the Dusp1 gene, and the Csnk2a1 gene in the second sample of blood is downregulated relative to the first sample of blood.

In some aspects, the method further comprises: comparing protein expression levels between the second sample of blood and the first sample of blood for at least one of the following protein biomarkers: Cathepsin L, ApoE, ARHGAP1, and TALDO1; classifying the vandefitemcel administration as effective in ameliorating the chronic condition caused by the stroke when at least one of the Cathepsin L protein biomarker, the ApoE protein biomarker, the ARHGAP1 protein biomarker, and the TALDO1 protein biomarker in the second sample of blood is downregulated relative to the first sample of blood.

In some aspects, the method further comprises: comparing a protein expression level between the second sample of blood and the first sample of blood for the protein biomarker Filamin A; and classifying the vandefitemcel administration as effective in ameliorating the chronic condition caused by the stroke when the protein biomarker Filamin A in the second sample of blood is upregulated relative to the first sample of blood.

In some aspects, administering the vandefitemcel further includes administering the vandefitemcel by intracerebral implantation.

In some aspects, administering the vandefitemcel further includes injecting the vandefitemcel at multiple sites within the region of the brain.

In some aspects, administering the vandefitemcel further includes administering the vandefitemcel stereotactically via a burr hole in a skull of the subject.

In some aspects, the region of the brain is a peri-stroke cortex.

In some aspects, administering the vandefitemcel further includes administering between about 1.0 million cells and 10.0 million cells.

In some aspects, the vandefitemcel is made by a method comprising: providing a culture of MSCs; contacting the culture of MSCs with the polynucleotide encoding the NICD, wherein the polynucleotide does not encode a full-length Notch protein, selecting cells that include the polynucleotide; and further culturing the selected cells in the absence of selection for the polynucleotide.

In some aspects, the MSCs are human bone marrow-derived cells.

In some aspects, the vandefitemcel is suspended in a sterile isotonic crystalloid solution.

In some aspects, the stroke is ischemic stroke and wherein the vandefitemcel is administered thirty days or more after the ischemic stroke.

In some aspects, a composition for treating chronic cortical hyperexcitability is disclosed. The composition comprising: vandefitemcel in an amount between about 1.0 million cells and 10.0 million cells, wherein the vandefitemcel is produced by modifying mesenchymal stem cells derived from bone marrow; and one or more pharmaceutically acceptable excipients, wherein the composition treats the chronic cortical hyperexcitability by at least one of: increasing production of gamma-aminobutyric acid (GABA) transporters 1 (GAT1) in the brain of a subject; and increasing production of brain-derived neurotrophic factors (BDNF) in the brain of the subject.

In some aspects, the composition treats the chronic cortical hyperexcitability by increasing the production of GAT1 in a peri-stroke cortex of the brain of the subject.

In some aspects, the composition treats the chronic cortical hyperexcitability by increasing the production of BDNF in at least one of a peri-stroke cortex, corpus callosum, a somatosensory thalamus, an internal capsule, a contralesional hemisphere, and an ipsilesional hemisphere of the brain of the subject.

In some aspects, the composition treats the chronic cortical hyperexcitability by increasing production of doublecortin-positive (DCX⁺) neuronal progenitor cells (NPCs) in the brain of the subject.

In some aspects, the composition treats the chronic cortical hyperexcitability by increasing production of oligodendrocyte precursor cells (OPCs).

In some aspects, the OPCs are oligodendrocyte transcription factor 2-positive (Olig2⁺) OPCs.

In some aspects, the OPCs are proliferating cell nuclear antigen-positive (PCNA⁺) OPCs.

In some aspects, the composition treats the chronic cortical hyperexcitability by increasing production of myelin basic protein (MBP) in a contralesional cortex of the subject.

In some aspects, the composition treats the chronic cortical hyperexcitability by increasing production of glial fibrillary acidic protein-positive (GFAP⁺) astrocytes in the brain of the subject.

In some aspects, the composition treats the chronic cortical hyperexcitability by increasing production of ionized calcium-binding adaptor molecule 1-positive (Iba1⁺) microglia in the brain of the subject.

In some aspects, the composition treats the chronic cortical hyperexcitability by inducing synaptogenesis in a peri-stroke cortex of the brain of the subject.

In some aspects, the composition treats the chronic cortical hyperexcitability by reducing glutamatergic synaptic vesicles in an ipsilesional region of a peri-stroke cortex of the brain compared to a contralesional region of the peri-stroke cortex.

In some aspects, the composition treats the chronic cortical hyperexcitability by reducing perineuronal nets in an ipsilesional region of a peri-stroke cortex of the brain compared to a contralesional region of the peri-stroke cortex.

In some aspects, the vandefitemcel is made by a process including: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells are human bone marrow-derived cells.

In some aspects, the mesenchymal stem cells are transiently-transfected with a plasmid vector including the polynucleotide encoding the NICD.

In some aspects, the one or more pharmaceutically acceptable excipients includes at least one of buffers, proteins, stabilizers, and preservatives.

In some aspects, the vandefitemcel is suspended in a sterile isotonic crystalloid solution.

In some aspects, the one or more pharmaceutically acceptable excipients comprises carriers or diluents.

In some aspects, a composition for reversing stroke-induced changes in whole-blood gene expression is disclosed. The composition comprising: vandefitemcel in an amount between about 1.0 million cells and 10.0 million cells, wherein the vandefitemcel is produced by modifying mesenchymal stem cells derived from bone marrow; and one or more pharmaceutically acceptable excipients, wherein the composition reverses the stroke-induced changes in whole-blood gene expression by at least one of: downregulating CD8a gene expression; downregulating CD8b gene expression; downregulating Uchl1 gene expression; downregulating Casp3 gene expression; downregulating Ube2s gene expression; downregulating Slc18a2 gene expression; and upregulating Fcgr2b gene expression.

In some aspects, the composition reverses stroke-induced changes in whole-blood gene expression by downregulating Scarf1 gene expression.

In some aspects, the composition reverses stroke-induced changes in whole-blood gene expression by downregulating Dusp1 gene expression.

In some aspects, the composition reverses stroke-induced changes in whole-blood gene expression by downregulating Csnk2a1 gene expression.

In some aspects, the vandefitemcel is made by a process comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells are human bone marrow-derived cells.

In some aspects, the mesenchymal stem cells are transiently-transfected with a plasmid vector including the polynucleotide encoding the NICD.

In some aspects, the one or more pharmaceutically acceptable excipients includes at least one of buffers, proteins, stabilizers, and preservatives.

In some aspects, the vandefitemcel is suspended in a sterile isotonic crystalloid solution.

In some aspects, the one or more pharmaceutically acceptable excipients comprises carriers or diluents.

In some aspects, a composition for reversing stroke-induced changes in serum protein expression is disclosed. The composition comprising: vandefitemcel in an amount between about 1.0 million cells and 10.0 million cells, wherein the vandefitemcel is produced by modifying mesenchymal stem cells derived from bone marrow; and one or more pharmaceutically acceptable excipients, wherein the composition reverses the stroke-induced changes in serum protein expression by at least one of: upregulating Filamin A protein expression; downregulating Cathepsin L protein expression; downregulating ApoE protein expression; downregulating ARHGAP1 protein expression; and downregulating TALDO1 protein expression.

In some aspects, the vandefitemcel is made by a process comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells are human bone marrow-derived cells.

In some aspects, the mesenchymal stem cells are transiently-transfected with a plasmid vector including the polynucleotide encoding the NICD.

In some aspects, the one or more pharmaceutically acceptable excipients includes at least one of buffers, proteins, stabilizers, and preservatives.

In some aspects, the vandefitemcel is suspended in a sterile isotonic crystalloid solution.

In some aspects, the one or more pharmaceutically acceptable excipients includes carriers or diluents.

In some aspects, a method of inducing endogenous production of brain-derived neurotrophic factors (BDNFs) is disclosed. The method comprising: administering vandefitemcel to a region of the brain of a subject, wherein the vandefitemcel are cells descended from mesenchymal stem cells (MSCs) transiently-transfected by a polynucleotide encoding a Notch intracellular domain (NICD).

In some aspects, the production of BDNF is increased in a peri-stroke cortex of the brain of the subject.

In some aspects, the production of BDNF is increased in a corpus callosum of the brain of the subject.

In some aspects, the production of BDNF is increased in a somatosensory thalamus of the brain of the subject.

In some aspects, the production of BDNF is increased in an internal capsule of the brain of the subject.

In some aspects, the production of BDNF is increased in a contralesional hemisphere of the brain of the subject.

In some aspects, the production of BDNF is increased in an ipsilesional hemisphere of the brain of the subject.

In some aspects, a composition for inducing endogenous production of brain-derived neurotrophic factors (BDNFs) is disclosed. The composition comprising: vandefitemcel in an amount between about 1.0 million cells and 10.0 million cells, wherein the vandefitemcel is produced by modifying mesenchymal stem cells derived from bone marrow; and one or more pharmaceutically acceptable excipients.

In some aspects, the production of BDNF is increased in a peri-stroke cortex of the brain of a subject.

In some aspects, the production of BDNF is increased in a corpus callosum of the brain of a subject.

In some aspects, the production of BDNF is increased in a somatosensory thalamus of the brain of a subject.

In some aspects, the production of BDNF is increased in an internal capsule of the brain of a subject.

In some aspects, the production of BDNF is increased in a contralesional hemisphere of the brain of a subject.

In some aspects, the production of BDNF is increased in an ipsilesional hemisphere of the brain of a subject.

In some aspects, the vandefitemcel is made by a process comprising: providing a culture of mesenchymal stem cells; contacting the culture of mesenchymal stem cells with a polynucleotide encoding a Notch intracellular domain (NICD), wherein the polynucleotide does not encode a full-length Notch protein, selecting cells that include the polynucleotide; and further culturing the selected cells in the absence of selection for the polynucleotide.

In some aspects, the mesenchymal stem cells are human bone marrow-derived cells.

In some aspects, the mesenchymal stem cells are transiently-transfected with a plasmid vector including the polynucleotide encoding the NICD.

In some aspects, the one or more pharmaceutically acceptable excipients includes at least one of buffers, proteins, stabilizers, and preservatives.

In some aspects, the vandefitemcel is suspended in a sterile isotonic crystalloid solution.

In some aspects, the one or more pharmaceutically acceptable excipients includes carriers or diluents.

### BRIEF DESCRIPTION OF THE DRAWINGS

FIG. 1 is a schematic diagram illustrating a photothrombotic (PT) stroke induced in the right primary somatosensory cortex (S1) of a Sprague Dawley rat (top left). FIG. 1 also illustrates an approximate location of the necrotic area caused by the stroke (top middle) and of the three sites where vandefitemcel was into the peri-stroke cortex or ischemic penumbra (top right).

FIG. 2A illustrate example local field potential (LFP) recordings in response to electrical stimulation (500 μA) of the white matter from a cortical slice of a Sprague Dawley rat from the stroke vandefitemcel group (treatment group) recorded at baseline (drug-free artificial cerebrospinal fluid (ACSF)), after N-methyl-D-aspartate receptor antagonist (APV) (125 μM) and 6,7-dinitroquinoxaline-2,3-dione (DNQX) application, and in presence of the sodium channel blocker tetrodotoxin (TTX) (1 μM). All three example recordings are from the same slice (n=10 sweeps averaged for each condition).

FIG. 2B are close-ups of LFP recordings evoked in layer 4 at stimuli currents f 150, 200, 300, 400, and 500 μA averaged from 10 sweeps for each stimulus intensity. The use of APV/DNQX (blockers of postsynaptic N-methyl-d-aspartate (NMDA) and alpha-amino-3-hydroxy-5-methyl-4-isoxazole propionic acid (AMPA) glutamatergic receptors) eliminated any postsynaptic response allowing the isolation of the presynaptic response which corresponds to axonal terminal potentials due to the direct electrical stimulation of the presynaptic axons. Increasing the stimulus current intensity in the presence of APV/DNQX produced a larger amplitude of the presynaptic response without any resultant postsynaptic response. After TTX application, the presynaptic response disappeared and only stimulus artifacts remained, and intensifying of stimulus did not change field recording under the TTX perfusion.

FIG. 2C is an overlay of the LFP traces shown in FIG. 2B isolating the presynaptic response (time window between −2 ms and −6 ms after the stimulus onset), and the postsynaptic response (after 6 ms up to 50 ms post-stimulation).

FIG. 2D illustrates current source density (CSD) profiles calculated from field potentials presented in FIG. 2A.

FIG. 2E illustrate CSD contour plots computed from the laminar CSD profiles from FIG. 2D. The interlaminar CSD components as a function of time and cortical depth (relative to the electrode grid). Under the +APV/DNQX perfusion the postsynaptic sources and sinks are eliminated and under the TTX perfusion both presynaptic and postsynaptic sinks and sources are abolished (pre- and postsynaptic CSDs).

FIG. 2F illustrates overlayed traces from CSD electrodes #2 and #9 showing the effects of APV/DNQX and TTX on the pre- and postsynaptic source and sink (Baseline CSD is depicted in black, +APV/DNQX in orange and +TTX in gray). All records are from the same preparation and comparable results were observed across preparations (n=4).

FIG. 3A (left) illustrates an experimental design of how the LFP recordings are made ex vivo and showing the placement of the recording array spanning all the layers in the peri-stroke cortex and the placement of the stimulation electrode in the white matter. FIG. 3A (right) is a digital photomontage reconstructing the location of the recording electrodes in the coronal NeuN-stained section of the recorded cortex (Si cortex, Bregma −2.5 mm).

FIG. 3B is a coronal NeuN-stained brain section showing the anatomical location of the recording electrode. Electrodes were placed as follows: electrodes 1-2 in layer 1; electrodes 3-5 in layers 2-3; electrodes 6-9 in layer 4; and electrodes 10-14 in layers 5-6.

FIG. 3C illustrates representative LFP responses to a 500 μA stimulation of white matter from representative sham vehicle (Sham Veh), stroke vehicle (Stroke Veh) and stroke vandefitemcel (Stroke SB) conditions.

FIG. 3D illustrates CSD traces derived from the LFP recording in FIG. 3C.

FIG. 3E illustrates responses colored/shaded differently to highlight sinks (darker shade, defined as a negative deflection) and sources (lighter shade, defined as a positive deflection) derived from the LFP recording in FIG. 3C.

FIG. 3F are graphs illustrating close-ups on CSDs from layers 2-3 (top) and layer 4 (bottom) from FIG. 3D to illustrate sinks and sources.

FIG. 3G illustrates a distribution of evoked presynaptic and postsynaptic sinks and sources generated from CSDs in layers 2-3, 4, and 5-6 in response to a 500 μA stimulation of white matter. Each dot is a result from one subject.

FIG. 4A are graphs showing presynaptic and postsynaptic LFPs in two example channels from layers 2-3 (top graphs) and layer 4 (bottom graphs). The inset graph (rightmost) illustrates an example trace showing the APV/DNQX-sensitive LFP component (postsynaptic), and the TTX-sensitive LFP component (presynaptic).

FIGS. 4B and 4C illustrate the distribution of evoked presynaptic and postsynaptic LFPs in response to a 500 μA stimulation of white matter. Each dot is a result from one subject. For each LFP electrode, the area of the presynaptic LFP and the postsynaptic LFP was calculated.

FIG. 5A illustrates CSD traces evoked by white matter stimulation of increasing intensity in representative sham vehicle (Sham Veh), stroke vehicle (Stroke Veh) and stroke vandefitemcel cells (Stroke SB) subjects. Arrows in the top graphs of FIG. 5A indicate the traces expanded in bottom graphs labeled “Channel 7” or “Channel 8.”

FIGS. 5B and 5C are graphs illustrating the amplitude of presynaptic sinks and sources generated from CSDs in layers 2-3, 4, and 5-6 as a function of the intensity of white matter stimulation.

FIGS. 5D and 5E are graphs illustrating the amplitude of postsynaptic sinks and sources generated from CSDs in layers 2-3, 4 and 5-6 as a function of the intensity of white matter stimulation.

FIG. 6A are graphs illustrating a distribution of evoked presynaptic and postsynaptic sinks and sources generated from CSDs in layers 2-3, 4, and 5-6 in response to a 500 μA stimulation of white matter. Each dot is a result of one subject.

FIG. 6B are graphs illustrating the area of the presynaptic and postsynaptic sinks and sources generated from CSDs in layers 2-3 as a function of the intensity of white matter stimulation from the channels.

FIG. 6C are graphs illustrating the area of the presynaptic and postsynaptic sinks and sources generated from CSDs in layer 4 as a function of the intensity of white matter stimulation from the channels.

FIG. 6D are graphs illustrating the area of the presynaptic and postsynaptic sinks and sources generated from CSDs in layers 5-6 as a function of the intensity of white matter stimulation from the channels.

FIG. 7A illustrates detection of vandefitemcel cells (human STEM101/121+; arrowheads) in cortical (top image) and subcortical (bottom image) locations one week after injection into the peri-stroke cortex (treatment was administered one month post-stroke).

FIG. 7B illustrates an annotated coronal brain section of a rat five weeks post-stroke indicating the ipsilesional regions of interest (ROIs) analyzed using immunofluorescence histology stains.

FIG. 7C are images representing synaptophysin (synaptic vesicle marker) staining in the peri-stroke region.

FIG. 7D are diagrams showing percentages of each ROI's area stained for synaptophysin in the sham vehicle (Sham Veh, squares), stroke vehicle (Stroke Veh, triangles) and stroke vandefitemcel (Stroke SB, circles) groups. Synaptophysin staining in each ROI showed a significant increase in layer 4 of the peri-stroke cortex in the stroke vandefitemcel group in comparison to vehicle treatment (with similar trends in the peri-stroke cortex overall, as well as in layers 5-6).

FIG. 8A are various graphs of size measurements of analyzed brain regions showing a significant shrinkage of the ipsilesional cortex in the stroke vehicle group in comparison to the sham vehicle group as a result of stroke.

FIG. 8B are images representing staining of NeuN⁺ neurons in the peri-stroke region.

FIG. 8C are diagrams showing percentages of each ROI's area stained for NeuN in the sham vehicle (Sham Veh, squares), stroke vehicle (Stroke Veh, triangles) and stroke vandefitemcel (Stroke SB, circles) groups.

FIG. 9A are images representing vesicular GABA transporter (VGAT) staining in the peri-stroke cortex.

FIG. 9B illustrates the results of the staining illustrated in FIG. 9A, by the percentage of area occupied by VGAT staining for each ROI in the sham vehicle (Sham Veh, squares), stroke vehicle (Stroke Veh, triangles) and stroke vandefitemcel (Stroke SB, circles) groups.

FIG. 9C are images representing staining of GABA transporter 1 (GAT-1) in the peri-stroke cortex.

FIG. 9D illustrates the results of the staining illustrated in FIG. 9C, by the percentage of area occupied by GAT-1 staining for each ROI.

FIG. 10A are images representing glutamic acid decarboxylase 67 (GAD67) staining in the peri-stroke region.

FIG. 10B illustrates the results of the staining illustrated in FIG. 10A, by the percentage of each ROI's area stained for GAD67 in the sham vehicle (Sham Veh, squares), stroke vehicle (Stroke Veh, triangles) and stroke vandefitemcel (Stroke SB, circles) groups.

FIG. 10C are images representing staining of parvalbumin-positive (PV+) GABAergic interneurons in the peri-stroke cortex.

FIG. 10D illustrates the results of the staining illustrated in FIG. 10C, showing that both stroke and vandefitemcel treatment did not significantly change the percentage area of PV+ staining in any of the analyzed ROIs.

FIG. 11A are images representing brain-derived neurotrophic factor (BDNF) staining in the peri-stroke region.

FIG. 11B illustrates the results of the staining illustrated in FIG. 11A, by the percentage of each ROI's area stained for BDNF in the sham vehicle (Sham Veh, squares), stroke vehicle (Stroke Veh, triangles), and stroke vandefitemcel (Stroke SB, circles) groups.

FIG. 11C are images representing staining of doublecortin (DCX) in the ipsilesional hemisphere.

FIG. 11D illustrates the results of the staining illustrated in FIG. 11C, measuring quantification of the percentage area of DCX staining in each ROI showing a significant stroke-induced increase in the ipsilesional internal capsule.

FIG. 12A are images representing VGluT2 staining in the peri-stroke cortex.

FIG. 12B illustrates the results of the staining illustrated in FIG. 12A, by the percentage of each ROI's area stained for VGluT2 in the sham vehicle (Sham Veh, squares), stroke vehicle (Stroke Veh, triangles) and stroke vandefitemcel (Stroke SB, circles) groups. Stroke significantly lowered the percentage area of VGluT2+ glutamatergic synaptic vesicles in layer 4 of the peri-stroke cortex, with a similar trend (p=0.055) in the ipsilesional somatosensory thalamus, and a trend (p=0.064) for VGlutT2 increase in the peri-stroke corpus callosum (where VGlutT2 levels are very low).

FIG. 12C are images representing staining of wisteria floribunda agglutinin (WFA⁺) perineuronal nets (PNNs) in the peri-stroke region.

FIG. 12D illustrates the results of the staining illustrated in FIG. 12C, via an analysis of the percentage of area occupied by WFA staining which labels PNNs in each ROT. The WFA⁺ PNN percentage area was significantly lower in the ipsilesional corpus callosum in the stroke vehicle group compared to the sham vehicle group. Vandefitemcel treatment did not change the WFA⁺ PNN percentage area. An analysis of the ipsilesional:contralesional ratio of the WFA⁺ PNN percentage area showed a significantly lower ratio in layer 4 of the peri-stroke in the stroke vandefitemcel group in comparison to the stroke vehicle group.

FIG. 13A are images representing oligodendrocyte transcription factor 2 (Olig2) and proliferating cell nuclear antigen (PCNA) staining in the peri-stroke region.

FIG. 13B illustrates the results of the staining illustrated in FIG. 13A, via an analysis of the density of proliferating oligodendrocyte precursor cells (OPCs) in each ROI in the sham vehicle (Sham Veh, squares), stroke vehicle (Stroke Veh, triangles) and stroke vandefitemcel (Stroke SB, circles) groups.

FIG. 13C are images representing stains for myelin basic protein (MBP) in the ipsilesional hemisphere.

FIG. 13D illustrates the results of the staining shown in FIG. 13C, by the percentage of each ROI's area stained for MBP in the three experimental groups.

FIG. 14A are images representing BDNF staining in the contralesional hemisphere in the location corresponding to the peri-stroke region in the ipsilesional hemisphere.

FIG. 14B illustrates the results of the staining illustrated in FIG. 14A, by the percentage of each contralesional ROI's area stained for BDNF in the sham vehicle (Sham Veh, squares), stroke vehicle (Stroke Veh, triangles) and stroke vandefitemcel (Stroke SB, circles) groups.

FIG. 14C are images representing MBP staining in the contralesional hemisphere in the location corresponding to the peri-stroke region in the ipsilesional hemisphere.

FIG. 14D illustrates the results of the staining illustrated in FIG. 14C, by the percentage of each contralesional ROI's area stained for MBP in the three experimental groups.

FIG. 15A are images representing glial fibrillary acidic protein (GFAP) staining in the peri-stroke region.

FIG. 15B illustrates the results of the staining illustrated in FIG. 15A, by the percentage of each ROI's area stained for GFAP in the sham vehicle (Sham Veh, squares), stroke vehicle (Stroke Veh, triangles) and stroke vandefitemcel (Stroke SB, circles) groups.

FIG. 15C are images representing stains for RECA1 and ki67 in the ipsilesional hemisphere.

FIG. 15D illustrates the results of the staining illustrated in FIG. 15C, by an analysis of the density of proliferating OPCs in each ROI in the three groups in the experiment. Stroke led to a significantly higher density of proliferating RECA1⁺ endothelial cells in the ipsilesional internal capsule.

FIG. 16A are images representing staining for ki67⁺ and GFAP⁺ in the peri-stroke region (arrowheads within the staining plots show examples for double-positive cells).

FIG. 16B illustrates the results of the staining illustrated in FIG. 16A, by the density of proliferating GFAP⁺ astrocytes in each ROI in the sham vehicle (Sham Veh, squares), stroke vehicle (Stroke Veh, triangles) and stroke vandefitemcel (Stroke SB, circles) groups.

FIG. 16C are images representing ionized calcium-binding adapter molecule 1 (Iba1) staining in the peri-stroke region.

FIG. 16D illustrates the results of the staining illustrated in FIG. 16C, showing that compared to the sham vehicle group, the stroke vehicle group Iba1⁺ percentage area was significantly higher the ipsilesional corpus callosum, and for the ipsilesional internal capsule a similar trend (p=0.060) was found.

FIG. 17A (left panel) shows the number of whole-blood genes significantly downregulated and upregulated in the stroke vandefitemcel group (Stroke_SB) compared to the stroke vehicle (Stroke_Veh) group on Day 3 (D3) and Day 7 (D7) after treatment or post-treatment. FIG. 17A (right panel) shows the same comparison between the stroke vehicle and sham vehicle groups. Data represents 7-10 rats per group on Day 3 and 9-11 rats per group on Day 7.

FIGS. 17B and 17C are volcano plots of differentially expressed genes for the stroke vehicle group compared to sham vehicle group (Sham_Veh) and stroke vandefitemcel group compared to stroke vehicle group for Day 3 and Day 7 (post-treatment). Downregulated genes are represented in darker shading and unregulated genes are represented in lighter shading. The x-axes show the log₂ fold-change (FC) of the differentially expressed genes and the y-axes the −log₁₀ of the p value.

FIGS. 17D and 17E display the topmost relevant Gene Ontology (GO), Kyoto Encyclopedia of Genes and Genomes (KEGG), and Reactome pathways for up-regulated and down-regulated genes in the differential gene expression (DGE) analysis at Day 7, respectively, in two comparisons: stroke vehicle vs. sham vehicle (FIG. 17D) and stroke vandefitemcel vs. stroke vehicle (FIG. 17E).

FIG. 17F illustrates a correlation of fold change between the stroke effect (comparison stroke vehicle vs. sham vehicle group) on the x-axis and the vandefitemcel effect (comparison stroke vandefitemcel vs. stroke vehicle) on the y-axis in genes that are nominally significant (p<0.05) for both effects after treatment one month post-stroke. The left panel of FIG. 17F shows results for Day 3 and the right panel of FIG. 17F shows results for Day 7.

FIG. 17G is a heatmap depicting cell-type specificity of enrichment signals. Downregulated (top rows) and upregulated (bottom rows) differentially expressed features show substantial enrichment for known blood cell type markers on Day 3 and Day 7 after treatment with vandefitemcel cells or vehicle one-month post-stroke.

FIG. 17H illustrates total numbers of significantly downregulated or upregulated serum proteins on Day 7 after treatment with vandefitemcel cells or vehicle one month post-stroke.

FIGS. 17I and 17J are volcano plots of differentially expressed proteins for stroke vehicle compared to the sham vehicle group (FIG. 17I) and stroke vandefitemcel group compared to the stroke vehicle group (FIG. 17J) on Day 7 post-treatment. Downregulated genes are represented in darker shading and unregulated genes are represented in lighter shading.

FIG. 17K illustrates a list of “rescued” proteins that were upregulated or downregulated by stroke and upregulated or downregulated by vandefitemcel treatment. The color shading indicates the log₂ of the FC.

FIG. 18A (left panel) is a plot with sample outlier removal performed at Day 3 and FIG. 18A (right panel) is a plot with sample outlier removal performed at Day 7 based on Z-scores of standardized network connectivity.

FIG. 18B illustrates a pathway analysis of differentially expressed whole-blood genes on blood collection Day 3 post-treatment, with stroke vehicle vs. sham vehicle graphs (top) and stroke vandefitemcel vs. stroke vehicle graphs (bottom). The contribution of upregulated or downregulated proteins to specific GO, KEGG, or Reactome terms are shown, respectively.

FIG. 18C illustrates the top 20 genes that were induced by stroke and rescued by vandefitemcel treatment in the peri-stroke cortex, showing Day 3 (left) and Day 7 (right) examples of stroke vandefitemcel vs. stroke vehicle groups.

FIG. 19A illustrates a clustering dendrogram of genes with gene dissimilarity based on topological overlap on Day 3.

FIG. 19B illustrates the number of genes per co-expression module detected on Day 3.

FIG. 19C illustrates significant modules associated with the comparison of the stroke vandefitemcel group vs. the stroke vehicle group on blood collection Day 3 post-treatment. Modules 22 and 27 are downregulated while module 25 is upregulated for the stroke vandefitemcel group.

FIG. 19D illustrates the topmost relevant GO, KEGG, WikiPathways (WP), and Reactome upregulated and downregulated pathways for significant co-expression modules (modules 22, 25, and 27). Selected pathways have a minimum of 3 associated genes (intersect >=3).

FIG. 19E illustrates a network of the most connected genes (“eigengenes”) in module 22.

FIG. 19F illustrates a network of the most connected genes (“eigengenes”) in module 25.

FIG. 19G illustrates a network of the most connected genes (“eigengenes”) in module 27.

FIG. 20A illustrates another clustering dendrogram of genes with gene dissimilarity based on topological overlap.

FIG. 20B illustrates the number of genes per co-expression module detected.

FIG. 20C illustrates significant modules associated with the comparison stroke hMSC-SB623 group (or stroke vandefitemcel group) vs. stroke vehicle group on blood collection Day 7 post-treatment.

FIG. 20D illustrates the topmost relevant GO, KEGG, WP, and Reactome upregulated and downregulated pathways for the significant co-expression modules (modules 3, 10, 12, 13, 14, 18, 22, 23, and 27). Selected pathways have a minimum of three associated genes (intersect >=3).

FIG. 20E illustrates networks of the most connected genes (“eigengenes”) in module 3.

FIG. 20F illustrates networks of the most connected genes (“eigengenes”) in module 10.

FIG. 20G illustrates networks of the most connected genes (“eigengenes”) in module 12.

FIG. 20H illustrates networks of the most connected genes (“eigengenes”) in module 13.

FIG. 20I illustrates networks of the most connected genes (“eigengenes”) in module 14.

FIG. 20J illustrates networks of the most connected genes (“eigengenes”) in module 18.

FIG. 20K illustrates networks of the most connected genes (“eigengenes”) in module 22.

FIG. 20L illustrates networks of the most connected genes (“eigengenes”) in module 23.

FIG. 20M illustrates networks of the most connected genes (“eigengenes”) in module 27.

FIG. 21A is a plot with a sample outlier removal performed based on Z-scores of standardized network connectivity for serum samples collected on Day 7 after treatment with vandefitemcel, sham vehicle, or stroke vehicle which were administered one month post-stroke.

FIG. 21B illustrates a clustering dendrogram of proteins with protein dissimilarity based on topological overlap.

FIG. 21C illustrates modules associated with the comparison of the stroke vandefitemcel group vs. the stroke vehicle group on blood collection Day 7 post-treatment. Module 12 is downregulated for the stroke vandefitemcel group.

FIG. 21D illustrates the topmost relevant GO downregulated pathways for the significant co-expression module 12. Selected pathways have a minimum of three associated genes (intersect >=3).

FIG. 21E illustrates a network of the most connected genes (“eigengenes”) in module 12.

FIGS. 22A to 22D illustrate various examples of whole-blood genes that were differentially expressed in the stroke vandefitemcel group compared to the stroke vehicle group and significantly correlated (p<0.05, R>0.6; exact values indicated in the graphs) with BDNF expression (% area) in the brain on post-treatment Day 7 in at least one of the analyzed ROIs in the stroke vandefitemcel group. On Day 3 (FIGS. 22A and 22B) post-treatment, Uchl1 and CD8a were downregulated in blood in the stroke vandefitemcel group (compared to the stroke vehicle group) and negatively correlated with Day 7 BDNF brain histology the ipsilesional corpus callosum and peri-stroke cortex L2-3, respectively, in the stroke hMSC-SB623 group, whereas Fcgr2b was upregulated in blood and positively correlated with Day 7 BDNF brain histology in the peri-stroke corpus callosum. On Day 7 (FIGS. 22C and 22D), Casp3, Ube2s, and Slc18a2 genes were downregulated and correlated negatively with BDNF protein expression in the ipsilesional corpus callosum in the stroke vandefitemcel group, whereas Reep6 and Snx8 were upregulated and positively correlated with BDNF in the ipsilesional corpus callosum.

FIG. 22E are diagrams illustrating the overlap between the post-treatment Day 3 (top two images) and Day 7 (bottom two images) downregulated/upregulated rescued whole-blood genes (genes that were upregulated/downregulated by stroke and downregulated by vandefitemcel treatment) and the whole-blood genes that were upregulated/downregulated by vandefitemcel cells (compared to vehicle treatment) on Day 3 or Day 7 and negatively/positively correlated with BDNF protein expression in the brain on Day 7 after treatment.

### DETAILED DESCRIPTION

### Definitions

The below terms are defined as follows for purposes of this disclosure.

The terms “administration” and “administering” refer to the physical introduction of a composition comprising a therapeutic agent to a subject, using any of the various methods and delivery systems known to those skilled in the art. For example, routes of administration for vandefitemcel can include intracerebral, intrathecal, or other parenteral routes of administration, for example by injection or infusion. The phrase “parenteral administration” as used herein means modes of administration other than enteral and topical administration, usually by injection and infusion. Administering can also be performed, for example, once, a plurality of times, and/or over one or more extended periods, and can be a therapeutically effective dose or a subtherapeutic dose.

The term “chronic cortical hyperexcitability” is used to refer to a persistent state of increased neuronal activity in the cerebral cortex, rendering neurons more prone to fire action potentials in response to stimuli and leading to abnormal brain function.

The term “contralesional” or “contralesional hemisphere” refers to a brain hemisphere on the opposite side of the ischemic injury or damage.

The term “focal ischemic stroke” is used to refer to a type of ischemic stroke where blood flow blockage specifically affects a localized area of the brain, leading to damage in that particular area of the brain.

The terms “implantation” and “transplantation” are used to denote the introduction of exogenous cells (e.g., vandefitemcel or SB623 cells) into a subject or patient. Exogenous cells can be autologous (i.e., obtained from the subject) or allogeneic (i.e., obtained from an individual other than the subject).

The term “ipsilesional” or “ipsilesional hemisphere” refers to a brain hemisphere on the same side as the ischemic injury or damage.

The terms “ischemic cortex,” “peri-stroke cortex,” “peri-injury cortex,” or “ischemic penumbra” is a hypo-perfused area or section of brain tissue immediately surrounding an ischemic core or infarct core. This area of brain tissue is at risk for irreversible damage but can still be potentially salvageable. Some studies have defined this area or section of brain tissue as a region of reduced cerebral blood flow (CBF) where CBF levels are reduced to between approximately 10 and 15 ml/100 g/min and approximately 25 mL/100 g/min (Fisher and Bastan, 2012).

An “isotonic crystalloid solution” is a solution containing water-soluble electrolytes. Such a solution contains the same amount of electrolytes as plasma.

“Mesenchymal cells” refer to cells of mesenchymal tissue (e.g., chondroblasts, chondrocytes, osteoblasts, osteocytes, adipocytes) and their precursors and include, for example, fibroblasts (e.g., human foreskin fibroblasts), MSCs (as defined herein) and cells derived from MSCs such as, for example, vandefitemcel, as defined herein.

“Mesenchymal stem cells” or “MSCs” refer to adherent, non-hematopoietic pluripotent cells obtained from bone marrow. These cells are variously known as mesenchymal stem cells, mesenchymal stromal cells, marrow adherent stromal cells, marrow adherent stem cells and bone marrow stromal cells. MSCs can also be obtained from, e.g., umbilical cord blood, adipose tissue, dental pulp, Wharton's jelly, and various types of connective tissue. MSCs can be obtained by selecting (e.g., by growth in culture) adherent cells (i.e., cells that adhere to tissue culture plastic) from bone marrow. To obtain MSC populations having a sufficient number of cells for use in therapy, populations of adherent cells are expanded in culture after selecting for adherence. Expansion in culture also enriches MSCs, since contaminating cells (such as monocytes) do not proliferate under the culture conditions. Exemplary disclosures of MSCs are provided in U.S. Patent Publication No. 2003/0003090 and the literature (Jiang et al., 2002). Methods for isolating and purifying MSCs can be found, for example, in U.S. Pat. No. 5,486,359 and the literature (Pittenger et al., 1999; Dezawa et al., 2001). Human MSCs are commercially available (e.g., BioWhittaker, Walkersville, Md.) or can be obtained from donors by, e.g., bone marrow aspiration, followed by culture and selection for adherent bone marrow cells. See, e.g., WO 2005/100552. MSCs can also be isolated from umbilical cord blood (Campagnoli et al., 2001; Erices et al., 2000). Additional sources of MSCs include, for example, adipose tissue, dental pulp, and Wharton's jelly.

A “neurodegenerative disorder,” “neurological disorder,” or “neurological disease” refers to disorders or diseases in which cells of the central nervous system (CNS) stop working, die, or are compromised in some manner. These types of diseases or disorders can include, but are not limited to, Alzheimer's disease, Parkinson's disease, Huntington's disease, amyotrophic lateral sclerosis (ALS), traumatic brain injury (TBI), ischemic stroke, hemorrhagic stroke, chronic stroke, or spinal cord injury.

The “Notch protein” (e.g., Notch 1 protein) is a transmembrane receptor, found in all metazoans, that influences cell differentiation through intracellular signaling. Contact of the Notch extracellular domain (e.g., the extracellular domain of the Notch 1 protein) with a Notch ligand (e.g., Delta, Serrate, Jagged) results in two proteolytic cleavages of the Notch protein, the second of which is catalyzed by a γ-secretase and releases the Notch intracellular domain (NICD) into the cytoplasm. In the mouse Notch protein, this cleavage occurs between amino acids gly1743 and val1744. The NICD translocates to the nucleus, where it acts as a transcription factor, recruiting additional transcriptional regulatory proteins (e.g., MAM, histone acetylases) to relieve transcriptional repression of various target genes (e.g., Hes 1). Additional details and information regarding Notch signaling can be found in the literature (Artavanis-Tsakonas et al., 1995; Mumm and Kopan, 2000; Ehebauer et al., 2006).

The term “pharmaceutically acceptable excipient” refers to an excipient for administration of a pharmaceutical agent. These “excipients” refer to any substance other than the active pharmaceutical agent. Examples of pharmaceutically acceptable excipients can include, but are not limited to, buffers, proteins, carbohydrates, oleochemicals, petrochemicals, stabilizers, preservatives, fillers, diluents, binders, viscosity agents, coatings, disintegrants, colorants, and lubricants.

The terms “vandefitemcel,” vandefitemcel cells,” “SB623,” “SB623 cells,” “SB,” “hMSC-SB623,” and “hMSC-SB623 cells” all refer to populations of cells obtained following transient expression of an exogenous Notch intracellular domain (NICD) in MSCs. For example, a population of vandefitemcel or SB623 cells can be obtained by transient transfection of MSCs with a plasmid vector comprising sequences encoding a NICD (e.g., from the human Notch 1 protein) but not encoding a full-length Notch protein followed by selection (e.g., with G418). The selected cells can be further cultured in a standard culture medium, optionally supplemented with a serum, in the absence of any added growth factors or differentiation factors (other than those which may be present in the serum, if serum is present in the culture medium). Vandefitemcel are derived from human (allogeneic) bone marrow MSCs (hMSCs) by transient transfection of hMSCs with NICD (e.g., the human Notch1 intracellular domain (NICD1)), followed by selection, and subsequent expansion. This process produces a cell population that is different than the parental MSCs (Aizman et al., 2009; Tate et al., 2010). Vandefitemcel, SB623 cells, or hMSC-SB623 cells have also been referred to as descendants of NICD transiently-transfected MSCs (“DNTT-MSCs”).

The term “transiently-transfected” refers to methods of introducing foreign DNA or RNA into cells for temporary expression of the introduced gene(s). Transient transfection influences short-term gene expression, lasting for a few hours to several days.

“Therapeutically effective” means the amount of an agent required to provide a meaningful patient benefit or promote disease regression evidenced by a decrease in severity of disease symptoms, an increase in frequency and duration of disease symptom-free periods, or a prevention of impairment or disability due to the disease affliction. The therapeutically effective amount of an agent can be evaluated using a variety of methods known to the skilled practitioner such as a practitioner in the field of neurology.

### Preparation of Vandefitemcel

Vandefitemcel are modified bone marrow-derived mesenchymal stromal/stem cells developed as an allogeneic cell therapy for chronic motor deficit after traumatic brain injury and stroke. Vandefitemcel cells are generated under manufacturing practices by transient transfection with a plasmid containing the human Notch-1 intracellular domain, which is lost quickly during subsequent cell expansion and passaging. The characterization of vandefitemcel has been previously described elsewhere in detail (Aizman et al., 2009; Tate et al., 2010). Briefly, after thawing, vandefitemcel cells are washed and formulated in the vehicle solution PlasmaLyte A (pH 7.4, Multiple Electrolytes Injection, Type 1, Baxter) to a final concentration of 2.0×10∝cells/μL.

In some variations, the vandefitemcel administered are allogeneic cells descended from mesenchymal stem cells transiently-transfected by a polynucleotide encoding a Notch intracellular domain (NICD). The cells can be made by a method comprising providing a culture of the mesenchymal stem cells, contacting the culture of mesenchymal stem cells with the polynucleotide encoding an NICD (where the polynucleotide does not encode a full-length Notch protein), selecting cells that comprise the polynucleotide, and further culturing the selected cells in the absence of selection for the polynucleotide. The mesenchymal stem cells can be human bone marrow-derived cells.

As previously discussed, vandefitemcel can be obtained from marrow-adherent stromal cells, also known as MSCs, by transiently expressing the intracellular domain of the Notch protein in the MSCs. Transient expression of the Notch intracellular domain (e.g., the NICD from the human Notch 1 protein) in an MSC can be sufficient to convert a population of MSCs into a population of vandefitemcel. Additional treatment with growth and/or differentiation factors is not required. Thus, a population of MSCs can be converted to a population of vandefitemcel by transient transfection of MSCs with a plasmid vector comprising sequences encoding an NICD (but not encoding full-length Notch protein), followed by selection for cells comprising the vector and further culture of the selected cells in serum-containing medium, in the absence of exposure to additional growth and/or differentiation factors. See, for example, U.S. Pat. Nos. 7,682,825; 8,945,919; and WO 2009/023251; the contents of which are incorporated herein by reference in their entireties for the purposes of describing isolation of mesenchymal stem cells and conversion of mesenchymal stem cells to vandefitemcel (also referred to as “neural precursor cells” and “neural regenerating cells” in those documents).

In this disclosure, any polynucleotide encoding a Notch intracellular domain (e.g., a plasmid vector) can be used, and any method for the selection and enrichment of transfected cells can be used. For example, MSCs can be transfected with a vector containing sequences encoding a Notch intracellular domain (e.g., the human Notch 1 intracellular domain) and also containing sequences encoding a selection marker (e.g., drug resistance; e.g., resistance to G418). In some instances, two plasmid vectors, one containing sequences encoding a Notch intracellular domain and the other containing sequences encoding a drug resistance marker, can be used for transfection of MSCs. In these instances, selection is achieved, after transfection of a cell culture with the vector or vectors, by adding a selective agent (e.g., G418) to the cell culture in an amount sufficient to kill cells that do not comprise the vector but spare cells that do. Absence of selection entails the removal of said selective agent or reduction of its concentration to a level that does not kill cells that do not comprise the vector. Following selection (e.g., for seven days) the selective agent can be removed and the cells can be further cultured (e.g., for two passages) in serum-containing culture medium.

It is also possible, depending on the nature of the selection marker and/or the concentration of the selective agent used, that not every cell that lacks a vector encoding a selection marker will be killed during the selection process. For example, a selective agent may inhibit growth of a cell not comprising the selection marker and, after removal of the selective agent, the cell may recover and resume growth.

Preparation of vandefitemcel thus involves transient expression of an exogenous Notch intracellular domain in an MSC. To this end, MSCs can be transfected with a plasmid vector comprising sequences encoding a Notch intracellular domain (e.g., the human Notch 1 intracellular domain) wherein said sequences do not encode a full-length Notch protein. All such sequences are known and readily available to those of skill in the art (Mumm and Kopan, 2000).

Similar information is available for Notch proteins and nucleic acids from additional species, including rat, Xenopus, Drosophila, and human (Weinmaster et al., 1991; Schroeter et al., 1998). Additional information can also be found in NCBI Reference Sequence No. NM_017167, SwissProt P46531, SwissProt Q01705, and GenBank CAB40733. The foregoing references are incorporated by reference in their entireties for the purpose of disclosing the amino acid sequence of the full-length Notch protein and the amino acid sequence of the Notch intracellular domain in a number of different species.

In some instances, vandefitemcel can be prepared by introducing, into MSCs, a nucleic acid comprising sequences encoding a Notch intracellular domain such that the MSCs do not express exogenous Notch extracellular domain. Such can be accomplished, for example, by transfecting MSCs with a plasmid vector comprising sequences encoding a Notch intracellular domain wherein said sequences do not encode a full-length Notch protein.

Additional details on the preparation of vandefitemcel or SB623 cells, and methods for making cells with properties similar to those of vandefitemcel or SB623 cells which can be used in the methods disclosed herein, can be found in the literature (Dezawa et al., 2004) and U.S. Pat. Nos. 7,682,825; 8,945,919; and 9,441,199, the contents of which are incorporated herein by reference in their entireties for the purposes of describing alternative methods for the preparation of, vandefitemcel, and for providing methods for making cells with properties similar to those of vandefitemcel.

The vandefitemcel used for the experiments disclosed herein were stored in liquid nitrogen. For cocultures, except where indicated, the vandefitemcel cells were thawed, washed, and plated directly. For glutaminase inhibition assays, vandefitemcel (or SB623) cells were cultured after thawing in minimum essential medium-alpha (a-MEM) (from CORNING, Corning, NY) supplemented with 10% of fetal bovine serum (FBS). In cocultures with neurons or for glutamate assay, vandefitemcel (or SB623) cells were kept in BrainPhys™ medium supplemented with NeuroCult™ SM1 neuronal supplement. To assess the levels of glutamate release by the vandefitemcel cells into conditioned medium, Gibco GlutaMAX™ (100×; 200 mM L-alanyl-L-glutamine from Thermo Fisher Scientific, Waltham, MA) was added (final concentration 2 mM) to vandefitemcel cells cultured in BrainPhys™ medium supplemented with NeuroCult™ SM1 neuronal supplement and levels of glutamate were assessed after two days.

### Transfection

Methods for introduction of exogenous DNA into cells (i.e., transfection), and selection of transfected cells, are known in the art (Brent, R., et al., 1994; Bardy, C. et al., 2015).

### Experimentational Protocols and Animal Models

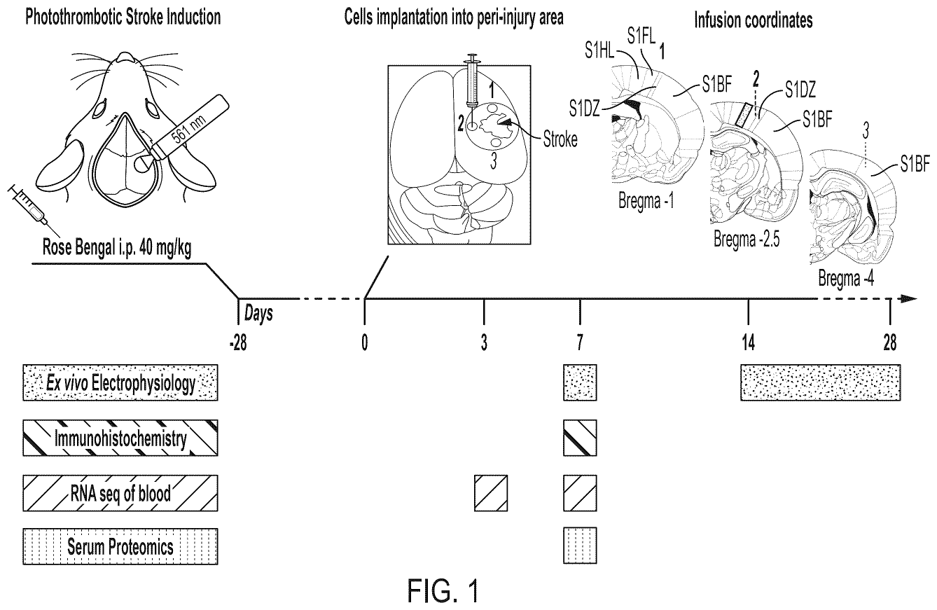

All experiments were conducted per protocols approved by the Institutional Animal Care and Use Committee at the University of California, San Francisco and the Gladstone Institutes. Precautions were taken to minimize stress and the number of animals used in each set of experiments. Adult, male Sprague Dawley rats from Charles River (CRL:CD(SD) #001) were used for the experiments. The rats weighed 70 g±10 g at the time of stroke/sham surgery. Two cohorts of animals were utilized to assess electrophysiological (cohort 1) and brain histology and peripheral blood (cohort 2) changes after cortical implantation of vandefitemcel cells in a chronic, focal ischemic stroke model as seen in FIG. 1. Each rat in the experiment received either a total dose of 1.8×10\hat{ }⁵ vandefitemcel cells in a 9 μL vehicle solution or 9 μL of the vehicle solution without cells.

Sprague Dawley rats are clinically established and translationally relevant models for investigating stroke treatments in human subjects (Biose, I. J., et al., 2022; Markgraf, Carrie G., et al, 1993).

FIG. 1 is a schematic diagram illustrating a photothrombotic (PT) stroke induced in the right primary somatosensory cortex (Si) of a Sprague Dawley rat (top left). FIG. 1 also illustrates an approximate location of the necrotic area caused by the stroke (top middle) and of the three sites where vandefitemcel was into the peri-injury cortical area (top right).

The rectangle shown in the infusion coordinates diagram in FIG. 1 (top right) illustrates the location of the multichannel Neuronexus probe used for columnar local field potential (LFP) recordings. The top right diagram also depicts coronal neocortical sections showing the sites of cell injections. The vandefitemcel cells were implanted 28 days (4 weeks) after stroke induction and the rats were euthanized for ex vivo electrophysiology (“cohort 1”) or immunohistochemistry and blood analyses (“cohort 2”). In cohort 1, ex vivo LFP was recorded at seven or 14-28 days after cell implantation. In cohort 2, blood was collected from live animals three days after cell implantation, then the animals underwent terminal blood collection and perfusion for immunohistochemical analysis at Day 7 post-cell implantation.

### Photothrombotic Cortical Stroke Induction

The Sprague Dawley rats were weighed and anesthetized with 2-5% isoflurane, after which they were placed in a stereotactic or stereotaxic frame. Photothrombosis (Paz et al., 2010; Paz et al., 2013) was performed after anesthesia. The Sprague Dawley rats were injected with a light-sensitive Rose Bengal dye (40 mg/kg) (Sigma-Aldrich) intraperitoneally and a 0.6 W light from a 3-mm-diameter Fiber-Lite MI-150 fiber optic cable was focused on the skull for 5 minutes. The optical system was designed to have an emission spectrum that encompassed the in vivo absorption range of Rose Bengal (maximum absorbance at 562 nm). To induce a focal photothrombotic ischemic stroke in the cortex, the light beam was centered using the following coordinates: −2.5 mm from bregma and 5.0 mm lateral from midline (target area: S1 Barrel Cortex). Sprague Dawley rats belonging to the control group received the same injection of Rose Bengal and identical anesthesia but were not photo-stimulated. All strokes were induced in the right hemisphere.

### Intra-Cortical Delivery of Vandefitemcel

One month post-stroke, randomly selected animals were anesthetized with 2-5% isoflurane, placed in a stereotactic or stereotaxic frame (Kopf Instruments), and implanted with vandefitemcel or a vehicle solution directly into the peri-injury cortex or peri-stroke cortex at three injection sites at the following stereotactic coordinates relative to bregma and dura: anterior injection site [antero-posterior (AP) −1.0 mm; mediolateral (ML) 4.0 mm, and dorsoventral (DV) 1.8 mm]; middle injection site [AP −2.5 mm, ML+3.5 mm, DV 1.4 mm] and posterior injection site [AP −4.0 mm, ML 5.0 mm, DV 1.3 mm]. Cell implantation or transplantation surgery was carried out within two (2) hours of thawing the vandefitemcel. A 26-gauge beveled needle attached to a manual microinjector (Kopf Instruments) was used for the infusion. The infusion rate was 1.0 μL/min. The total injected volume was 9 μL per Sprague Dawley rat (3 μL per injection site).

### Brain Slice Preparation in Rats

The Sprague Dawley rats were anesthetized with 5% isoflurane and perfused with ice-cold sucrose buffer containing (in mM): 234 sucrose, 2.5 KCl, 1.25 NaH₂PO₄, 10 MgSO₄, 0.5 CaCl₂, 26 NaHCO₃ and 11 glucose, equilibrated with 95% O₂ and 5% CO₂. The Sprague Dawley were decapitated, the brains of the rats were extracted, and coronal slices (400 m) containing the ischemic cortex were collected (n=2-4 slices per animal) using a Leica VT1200S vibratome and were placed in a humidified, oxygenated interface chamber and perfused at a rate of 2 mL/min at 34° C. with oxygenated artificial cerebrospinal fluid (ACSF) prepared as described above and supplemented with 300 M glutamine for cellular metabolic support (Ritter-Makinson et al., 2019; Cho et al., 2017; Ferguson et al., 2023).

### Extracellular Recordings of Cortical Local Field Potentials

Extracellular LFP recordings were obtained with a linear 16-channel multi-electrode array with 100 m spacing between the electrodes (Neuronexus, A16×1-2 mm-100-703) placed in the peri-stroke cortex (or the corresponding cortical region in the control group of rats, see FIG. 1). The orientation of the probe is such that the first electrode (e.g., #1) is placed in layer 1 of the cortex and the last electrode (e.g., #16) is placed at the bottom of layer 6/white matter. Electrode anatomical locations were as follows: 1-2 in layer 1; 3-5 in layers 2-3; 6-9 in layer 4; and 10-14 in layers 5-6. Electrodes #15 and 16 are localized in white matter (and were excluded from the analysis). To evoke synaptic responses, electrical stimulation is delivered in the white matter with a concentric bipolar tungsten electrode (50-100 kΩ, FHC Inc.) at intensities increasing from 10 μA to 600 μA (10 intensities total). Stimulation pulses (100 μs duration) were delivered every 8 seconds and were repeated 10-20 times in a single recording. Signals from all sixteen channels were digitized at 24.414 kHz, band-pass filtered between 100 Hz and 6 kHz, amplified at 10,000×, and stored using the RZ5 processor workstation (Tucker-Davis Technologies). To distinguish the presynaptic and postsynaptic components of the evoked response, after baseline recordings in ACSF, blockers of glutamatergic synapses were added, 6,7-dinitroquinoxaline-2,3-dione (DNQX, 10 μM; Sigma), which is an antagonist of alpha-amino-3-hydroxy-5-methyl-4-isoxazole propionic acid (AMPA) receptors and 2-amino-5-phosphonopentanoic acid (APV, 125 μM, Sigma), which is an antagonist of N-methyl-d-aspartate (NMDA) receptors, and continued recording for 10 minutes while increasing the intensity of the stimulus from 150 μA to 500 μA. The perfusion was then switched back to ACSF. After a 30-minute ACSF wash, the sodium channel blocker tetrodotoxin (TTX 1 μM) was perfused to abolish the action potential discharges, and recordings were continued for 10 minutes with increasing stimuli up to 500 μA. The concentration of each drug was chosen based on previous reports (Nugent et al., 2008; Mikulec et al., 1998). The APV/DNQX-sensitive response was identified as postsynaptic, and the remaining TTX-sensitive response was defined as presynaptic. To better examine the location, direction, and strength of currents evoked in response to electrical stimulation, a current source density (CSD) analysis was performed by calculating the second spatial derivative of the LFP (Ferguson et al., 2023; Freeman and Nicholson, 1975). When net positive current enters a cell, this creates an extracellular negativity that is reflected in a current sink, and appears as a negative deflection in the CSD. In contrast, current sources indicate net negative current flowing into a cell and appear as a positive deflection in the CSD. To facilitate visualization of CSD profiles along the depth axis (cortical layers), color image plots with blue and red were generated representing current sinks and sources respectively. LFP and CSD plots were generated using MATLAB scripts kindly provided by the Huguenard lab (see Ferguson et al., 2023).

Analysis of Local Field Potential Responses and Current Source Density with Exclusion Criteria

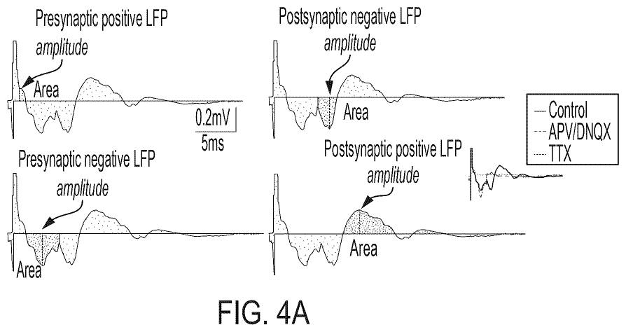

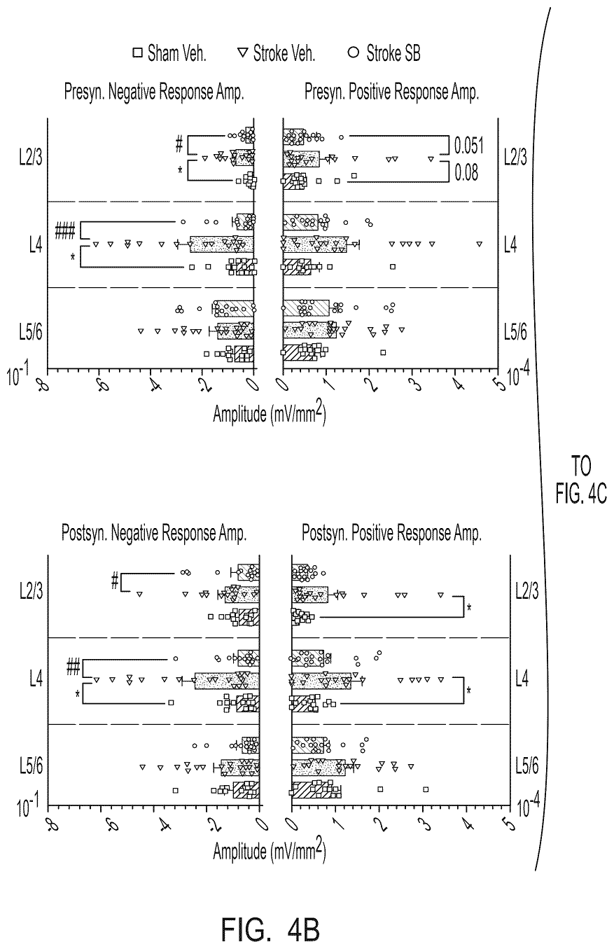

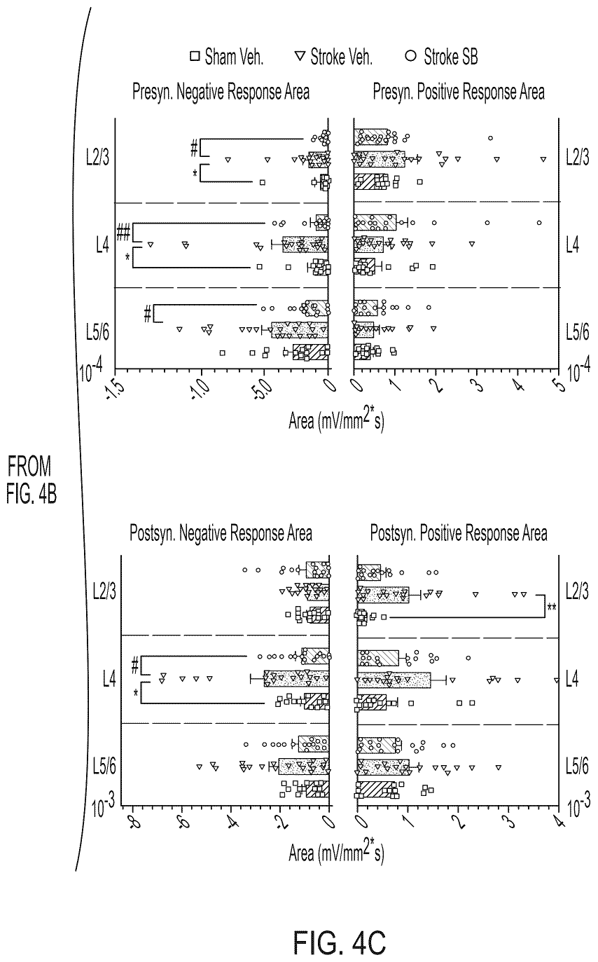

LFP signals were imported into MATLAB software, version R2019a and processed using a combination of custom and modified routines from the freely available MATLAB packages. At each stimulus intensity, 10-20 recorded sweeps were averaged from each channel and each slice after excluding sweeps with occasional noise and small signal-to-noise ratio. The presynaptic response was measured between 0.3 and 6 ms after stimulation, and the postsynaptic response from 6 ms up to 50 ms. The amplitude of the negative and positive components of presynaptic and postsynaptic responses were analyzed by measuring the baseline-to-peak value; and the area above or below the curve (as seen in FIGS. 4A-4C). Presynaptic and postsynaptic responses obtained from electrodes 3-5 were averaged and expressed as LFP of layers 2-3; presynaptic and postsynaptic responses obtained from electrodes 7-9 were averaged and expressed as LFP of layer 4; and presynaptic and postsynaptic responses obtained from electrodes 10-12 were averaged and expressed as LFP of layers 5-6. LFP channels with noise were excluded from the analysis.

The one-dimensional current source density (CSD) profiles were calculated as described by Ferguson et al., 2023. Since signals from adjacent electrodes constitute LFPs separated by 0.1 mm in the cortex, the CSD is calculated as the second derivative over space using adjacent electrodes. (Additional information can be found in Hodgkin and Huxley, 1952; Mitzdorf, 1985). In cases where LFPs had to be excluded, an assumed LFP was recovered by linearly interpolating between the LFPs from the two adjacent electrodes.

Presynaptic and postsynaptic responses obtained from electrodes 3-5 were averaged and expressed as CSD of layers 2-3; presynaptic and postsynaptic responses obtained from electrodes 7-9 were averaged and expressed as CSD of layer 4; and presynaptic and postsynaptic responses obtained from electrodes 10-12 were averaged and expressed as CSD of layers 5-6. For statistical analysis, n=12-15 animals per group were included (1-2 slices per animal).

### Brain Tissue Preparation for Histology

The Sprague Dawley rats were anesthetized seven days post-treatment with 2-5% isoflurane inhalation and transcardially perfused with phosphate-buffered saline (PBS) followed by 4% paraformaldehyde in PBS using a peristaltic pump. The brains of the rats were removed and post-fixed in 4% paraformaldehyde in PBS for at least 24 hours at 4° C., then the 4% paraformaldehyde solution was replaced with PBS and the brains were stored at 4° C.

### Tissue Embedding and Sectioning

Fixed brains were processed at NeuroScience Associates (NSA, Knoxville, TN) using their Multibrain Technology (NeuroScience Associates, Knoxville, TN). Up to 16 rat brains were randomly assigned to and embedded in a gelatin matrix block in a coronal sectioning configuration, frozen, and sectioned into 30 μm thick slices with a sliding microtome. Multibrain sections were collected in a cycled series of 24 cups, submerged in antigen preserve solution and shipped back for immunohistochemistry to SanBio, Inc.

### Immunostaining for Histology

Free-floating sections were washed with TBS, incubated in pre-heated Citrate Buffer in an 80° C. water bath for antigen retrieval for 20 minutes, washed with TBS, followed by 1 hour blocking in 10% Normal Donkey Serum (NDS) diluted in Fish Skin Gelatin Buffer solution (FSGBT: 1% Bovine Albumin Serum (BSA), 0.3% Triton X-100, 0.2% fish skin gelatin, in TBS) at room temperature (RT). Sections were incubated overnight at 4° C. with the primary antibodies (see Table 1) in 3% NDS diluted in FSGBT solution. On the subsequent day, sections were washed with TBS and incubated at RT for 1 hour with the appropriate secondary antibodies (see Table 1) in 3% NDS diluted in FSGBT solution, and counterstained with DAPI nuclear label (1:2000, Millipore Sigma, D9542). Sections were washed with TBS and mounted on gelatin-coated slides (NeuroScience Associates, Knoxville, TN) and cover-slipped with ProLong™ Gold Antifade Mountant media (Invitrogen, P36930) and left to dry at RT in a dark environment overnight.

The STEM101/121 DAB staining to detect vandefitemcel cells, in a set of serial images that allowed analysis of every 32nd serial section in the whole brain, was done by the service lab NeuroScience Associates (NSA, Knoxville, TN) according to their protocols.

TABLE 1
Primary and Secondary Antibodies

| Primary Antibody | Secondary Antibody |
|---|---|
| BDNF (1:300, guinea | Donkey anti-guinea pig AF 488 (1:1000, |
| pig, Alomone, AGP- | Jackson ImmunoResearch, 706-545-148) |
| 021) | |
| DCX (1:1000, rabbit, | Donkey anti-rabbit AF 647 (1:1000, Jackson |
| Cell Signaling, 4604S) | ImmunoResearch, 711-606-152) |
| GAD67 (1:250, mouse, | Donkey anti-mouse AF 647 (1:1000, Jackson |
| Millipore Sigma, | ImmunoResearch, 715-605-151) |
| MAB5406) | |
| GAT-1 (1:200, rabbit, | Donkey anti-rabbit AF 647 (1:1000, Jackson |
| Alomone, AGT-001) | ImmunoResearch, 711-606-152) |
| GFAP (1:5000, chicken, | Donkey anti-chicken AF 488 (1:1000, |
| BioLegend, 829401) | Jackson ImmunoResearch, 703-545-155) |
| GFAP (1:5000, chicken, | Donkey anti-chicken AF 488 (1:1000, |
| Invitrogen, PA1-2004) | Jackson ImmunoResearch, 703-545-155) |
| Ibal (1:1000, goat, | Donkey anti-goat AF 594 (1:1000, Jackson |
| abcam, ab5076) | ImmunoResearch, 705-585-147) |
| | Donkey anti-goat AF 488 (1:1000, Jackson |
| | ImmunoResearch, 705-546-147) |
| Ki67 (1:800, rabbit, | Donkey anti-rabbit AF 555 (1:1000, |
| Abcam, AB15580) | Invitrogen, A32794) |
| Ki67 (1:800, rabbit, | Donkey anti-rabbit AF 594 (1:1000, Jackson |
| Cell Signaling, 9129) | ImmunoResearch, 711-585-152) |
| MBP (1:1000, mouse, | Donkey anti-mouse AF 647 (1:1000, Jackson |
| R&D Systems, | ImmunoResearch, 715-605-151) |
| MAB42282) | |
| NeuN (1:2000, guinea | Donkey anti-guinea pig AF 488 (1:1000, |
| pig, Millipore Sigma, | Jackson ImmunoResearch, 706-545-148) |
| ABN90) | |
| Olig2 (1:400, goat, | Donkey anti-goat AF 647 (1:1000, Jackson |
| R&D Systems, AF2418) | ImmunoResearch, 705-606-147) |
| | Donkey anti-goat AF 488 (1:1000, Jackson |
| | ImmunoResearch, 705-546-147) |
| PCNA (1:500, mouse, | Donkey anti-mouse AF 594 (1:1000, Jackson |
| Cell Signaling, 2586S) | ImmunoResearch, 715-586-151) |
| PV (1:3000, chicken, | Donkey anti-chicken AF 594 (1:1000, |
| EnCor, CPCA-Pvalb) | Jackson ImmunoResearch, 703-585-155) |
| RECA1 (1:600, mouse, | Donkey anti-mouse AF 647 (1:1000, Jackson |
| Abcam, AB9774) | ImmunoResearch, 715-605-151) |
| STEM101 (1:500 mouse, | Horse anti-mouse biotinylated |
| Takara, Y40400) | (1:500 Vector, BA-2001); chromogen NiDAB |
| | black |
| STEM121 1:500, mouse, | Horse anti-mouse biotinylated |
| Takara, Y40410) | (1:500 Vector, BA-2001); chromogen NiDAB |
| | black |
| Synaptophysin | Donkey anti-mouse AF 555 (1:1000, |
| (1:2000, mouse, Cell | Invitrogen, A32773) |
| Signaling, 9020S) | |

### Microscopy Imaging

Immunofluorescent images were acquired with a 10× objective and stitched to composite whole-brain coronal images with Leica DMi8 microscope and Leica LAS X software. All images of a specific marker combination were acquired with the same imaging settings.

Brightfield images for STEM101/121 were acquired with a 20× objective and stitched to composite whole-brain coronal images with an Olympus VS200 Research Slide Scanner (Olympus/Evident, Center Valley, PA) with a Hamamatsu Orca-Fusion camera (Hamamatsu Photonics, Skokie, IL). Individual images were created with the OlyVIA Viewer software (Olympus/Evident, Center Valley, PA). Imaging was performed by the University of Chicago Integrated Light Microscopy Core Facility using consistent settings for all images in the experiment.

### Image Analysis

All image analysis was done fully blinded to the treatment groups. Tracing of regions of interest (ROIs) and image analysis was performed using Fiji/ImageJ software. The following ROIs were traced for each rat in both hemispheres (in sections ranging approx. from anterior-posterior (AP) −1.92 mm to AP −4.36 mm coordinates according to Paxinos and Watson's rat brain atlas, 7th edition): whole cortex, whole corpus callosum, and hippocampal dentate gyrus. In addition, the somatosensory thalamus and internal capsule, as well as peri-stroke ROIs which contained the area in which the multielectrode arrays, were placed for electrophysiology recordings in previous experiments were traced (in sections ranging approx. from AP −1.92 mm to AP −4.08 mm). The peri-stroke ROIs were designed to match the layers probed by the multielectrode in the electrophysiology experiments and subdivided as follows: peri-stroke cortex (spanning all cortical layers), peri-stroke cortical layers 2-3, layer 4 and layers 5-6, and peri-stroke corpus callosum. For each immunofluorescent marker combination, at least 3 multi-brain sections in the AP target range (containing 3 sections per rat in the experiment) were stained.

For each immunohistology marker, all images that met the location and quality inclusion criteria were analyzed with the same ImageJ macro, which generated automated thresholds for percentage of stained area and which also measured the co-localization of cell type-specific markers with proliferation markers (PCNA or ki67) and density of double-positive cells where applicable. To assess loss of brain volume after stroke, the size of the traced ROIs was analyzed in the MBP staining in at least two sections per rat within the target region for each ROI. Midline thickness measurements (in the MBP staining) and STEM101/121+ vandefitemcel counts (using strict criteria for color and morphology of the cells) were done manually in the Fiji/ImageJ software. To estimate the total number of STEM101/121+ vandefitemcel cells in the brain, all STEM101/121+ cells were counted in serial sections representing a 1/32 of the brain, then the resulting cell count was multiplied by 32.

### Statistical Analysis of Electrophysiology Data and Histology Results

Statistical analyses were done using Graphpad Prism (version 10.1.0). First, a Grubb's outlier test was done to exclude maximal one outlier per group, then a Shapiro-Wilk test was used to test for normal distribution. This was followed either by a one-way ANOVA with Sidak's multiple comparisons test for normally distributed data or by a Kruskal-Wallis test combined with Dunn's multiple comparisons test. Adjusted p-values for the two planned comparisons (stroke vandefitemcel vs. stroke vehicle; and stroke vehicle vs. sham vehicle; for electrophysiology data comparison between indicated groups was done for each stim separately) are reported in the graphs.

For all analyses, values of p<0.05 were considered significant, and p values <0.1 are reported as trends. The specific n for each graph can be found in the figure legends. Numerical values are given as mean±standard error of the mean (SEM) unless stated otherwise.

### Blood Collection

Blood was collected at two time points: on Day 3 and Day 7 after treatment or post-treatment, which was administered one-month post-stroke. On Day 3, rats were anesthetized with isoflurane inhalation during blood collection from the femoral artery, and 100 μL was allocated into the RNAprotect Animal Blood Tube (Qiagen, #76544, Germantown, MD, USA) for RNA isolation (stored at −20° C.). On Day 7, with a syringe, blood was collected from the left ventricle/atrium of the heart right before transcardial perfusion. 500 μL was allocated into the RNAprotect Animal Blood Tube (Qiagen, #76554, Germantown, MD, USA) for RNA isolation (stored at −20° C.) and approximately 1,500 μL was allocated into a separate microcentrifuge tube for serum preparation.

### Serum Preparation

Blood samples from Day 7 post-treatment were left at RT for at least 20 minutes and then centrifuged at 1.6× g for 12 minutes. Serum was collected, aliquoted, and transferred into clean tubes and stored at −80° C.

### RNA Isolation

Total RNA was isolated utilizing the RNeasy Protect Animal Blood Kit (Qiagen, #73224, Germantown, MD, USA) and performed according to manufacturer instructions. Blood samples collected in the RNAprotect Animal Blood Tubes were thawed overnight at RT and centrifuged, the supernatant was discarded and the pellet washed with RNase-free water and centrifuged, and then dissolved with buffer solution. Samples were digested with proteinase K on an incubating shaker followed by homogenization through spin columns and then isolation of the supernatant. RNA samples were bonded to another type of spin column followed by digestion of DNA and washed with buffer solutions. RNA samples were released from spin columns, checked for RNA concentrations on a NanoDrop spectrophotometer with NanoDrop software (NanoDrop Technologies, Inc, #NC-1000, Wilmington, DE, USA), aliquoted, and stored at −80° C.

### RNA Sequencing

RNA sequencing was done at Novogene Co., Ltd. In short, globin mRNA was depleted using an Invitrogen GLOBINclear kit according to manufacturer instructions. After checking RNA quality, purity, and integrity, a total amount of 1 g RNA for each sample was used for the following steps. Sequencing libraries were generated using NEBNext Ultra™ RNA Library Prep Kit for Illumina (NEB, USA) and index codes were added for sample identification. Library fragments were purified with the AMPure XP system (Beckman Coulter, Beverly, USA). PCR was performed with Phusion High-Fidelity DNA polymerase, universal PCR primers and Index (X) Primer, followed by purification (AMPure XP system) and assessment of library quality on the Agilent Bioanalyzer 2100 system. After cluster generation on a cBot Cluster Generation System using PE Cluster Kit cBot-HS (Illumina), the library preparations were sequenced to a depth of 20 M reads per sample on an Illumina NovaSeq6000 platform and paired-end reads (length 150 bp) were generated. Raw FASTQ reads were processed using fastp, to obtain clean reads and check data quality.

Reference genome and annotation files were downloaded directly from NCBI/UCSC/Ensembl. HISAT2 software was used for mapping paired-end clean reads to the reference genome. Stringtie and gffcompare were used for novel gene prediction. Read numbers for each mapped gene were quantified using Featurecounts before calculating RPKM of each gene.

### RNA-Seq Quality Control

Expressed genes were defined as genes with FPKM>0.5 in at least 80% of samples from each group (sham vehicle, stroke vehicle, and stroke vandefitemcel group) and time point (Day 3 and Day 7 post-treatment). Expressed genes were included into the analysis. A total of 10,194 and 10,618 expressed genes from D3 and D7 respectively were used in the downstream analysis.

### Differential Gene Expression

Differential Gene Expression (DGE) analyses were performed using DESeq2. The RNA Integrity Number (RIN) was included as a covariate in the model as follows: RIN+Group where ‘Group’ represents all the different conditions of the study. Genes with p values <0.05 were considered as significantly differentially expressed. The biomaRt (Durinck et al., 2005) v3.12 package in R with the GENECODE V19 annotation was used to extract gene names, gene biotypes, and gene descriptions.

### Enrichment Analysis of Gene Sets

Enrichment for Gene Ontology (GO; biological process, cellular component, and molecular function), Kyoto Encyclopedia of Genes and Genomes (KEGG) pathways, WikiPathways (WP) and Reactome were performed using the gprofiler2 R v0.2.0 package (Raudvere et al., 2019). Background was restricted to the expressed set of genes. Only pathways containing less than 1,000 genes were assessed. An ordered query was used, ranking genes by FDR-corrected p-value for DGE analyses.

### Co-Expression Analysis

Network analysis was performed with the Weighted Gene Correlation Network Analysis (WGCNA) (Langfelder and Horvath, 2008) package using signed networks. A soft-threshold power of 22 in D3 and 7 in D7 groups was used to achieve approximate scale-free topology (R2>0.8). The ‘blockwiseModules’ function was used to construct the networks. The network dendrogram was created using average linkage hierarchical clustering of the topological overlap dissimilarity matrix (1-TOM). The hybrid dynamic tree-cutting method was used to define modules. Modules were summarized by their first principal component (ME, module eigengene), and modules with eigengene correlations >0.9 were merged together. Modules were defined using biweight midcorrelation (bicor), with a minimum module size of 10. Genes outside of any modules (indicating low co-expression) were combined together in a grey module. Genes within each module were ranked based on their module membership (kME), defined as correlation to the module eigengene. Detected modules were correlated to the groups under study.

### Cell Type Enrichment Analysis

Differentially expressed genes were employed to conduct cell type enrichment analysis. The g:Orth function from the g:Profiler package was utilized to identify orthologous genes between the rat and human genomes. These human Ensembl orthologous gene identification numbers were subsequently employed to perform cell type enrichment analyses using the R package ‘clustermole’ v.1.1.0 (Dolgalev, 2021). Tissue types encompassing blood, blood vessels, and the immune system were focused on. Following filtering, CellMarker genes were selected for visualization.

### Serum Proteomics Analysis

A semi-quantitative analysis of serum proteins was done at RayBiotech Life, Inc., with the biotin-label-based antibody array RayBio Rat L1500. Differential protein expression was calculated by fitting a liner model for each of the 1,500 proteins, using the lm function from the ‘stats’ package (version 4.2.2) in R. Pathway enrichment analyses were performed as described previously for differential gene expression (DGE) with the background restricted to the 1,500 proteins.

### Correlation Analysis Between Differentially Expressed Whole-Blood Genes/Serum Proteins and BDNF Brain Histology

Spearman's correlations were calculated between the percentage area of BDNF staining in all analyzed regions of interest (ROIs) in brain tissue collected for histology on Day post-treatment and the log₁₀(FPKM) whole-blood RNA expression values on post-treatment Day 3 and Day 7 or serum protein expression results on Day 7 for each group without exclusion of any outliers. Correlations with p<0.05 and absolute R>=0.5 were considered significant.

### REPRESENTATIVE EXAMPLES, EXPERIMENTS, AND RESULTS

The following representative examples, experiments, and results discussed below are only for illustrative purposes and are not intended to limit or define the entire scope of this disclosure. It should be appreciated that the disclosure is not limited to the particular examples or embodiments described and illustrated herein but includes all modifications and variations falling within the scope of this disclosure.

### Vandefitemcel Reduces Chronic Cortical Hyperexcitability

Chronic cortical hyperexcitability has been associated with maladaptive outcomes in patients after stroke such as motor spasticity and epilepsy (Medalla et al., 2020; Li, 2017; Olsen et al., 1987; Mishra et al., 2022). Chronic cortical hyperexcitability can also be associated with other neurological disorders or diseases including Amyotrophic Lateral Sclerosis (ALS) and traumatic brain injury (Menon et al., 2020; Koenig et al., 2019).

As previously discussed, adult Sprague Dawley rats were subjected to photothrombotic (PT) stroke or sham surgery and the effects of vandefitemcel cell transplants on cortical network excitability at chronic time points (>1 month) after stroke were measured. This stroke animal model was chosen because it causes consistent and highly reproducible ischemic injuries in the cortex (reviewed, for example, in: Labat-gest and Tomasi, 2013; Uzdensky, 2018). Also, Sprague Dawley rats were chosen because they are clinically established and translationally relevant models for investigating stroke treatments in human subjects (Biose, I. J., et al., 2022; Markgraf, Carrie G., et al, 1993).

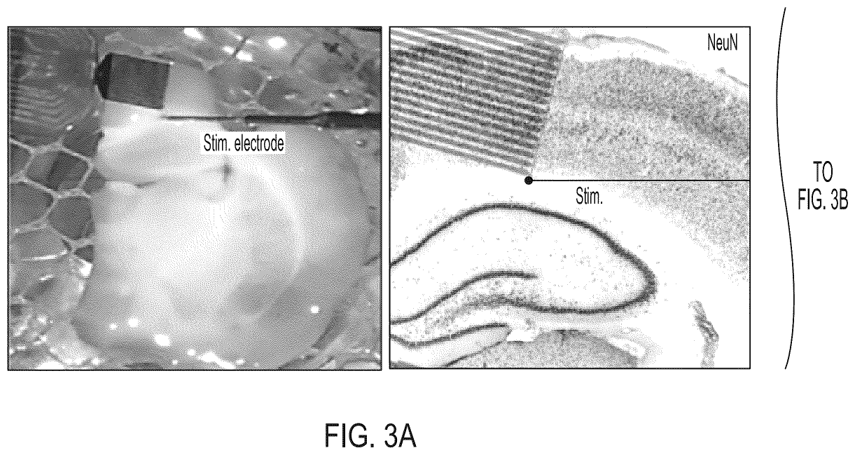

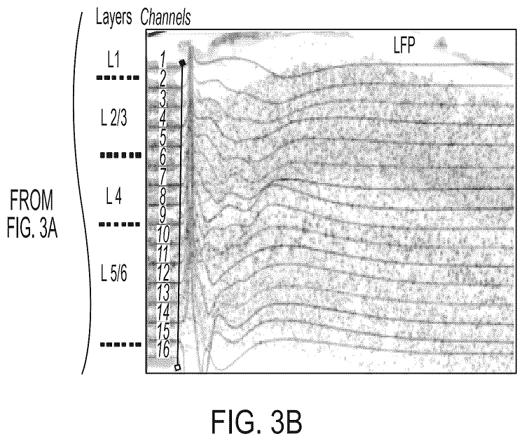

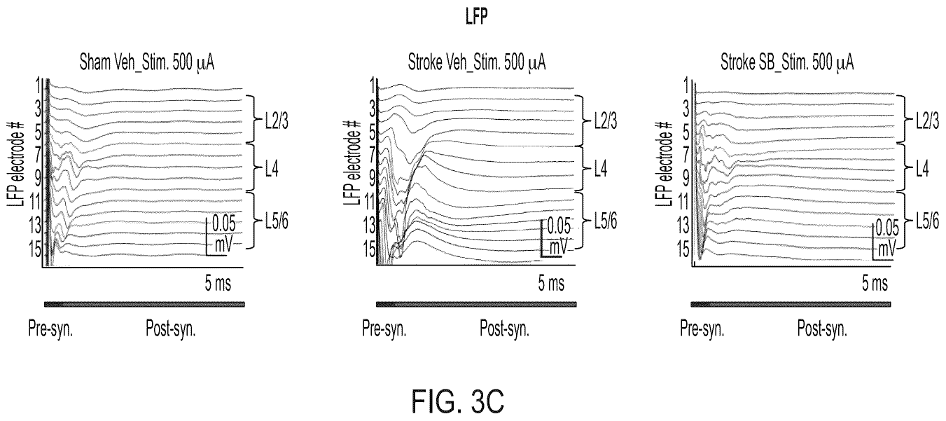

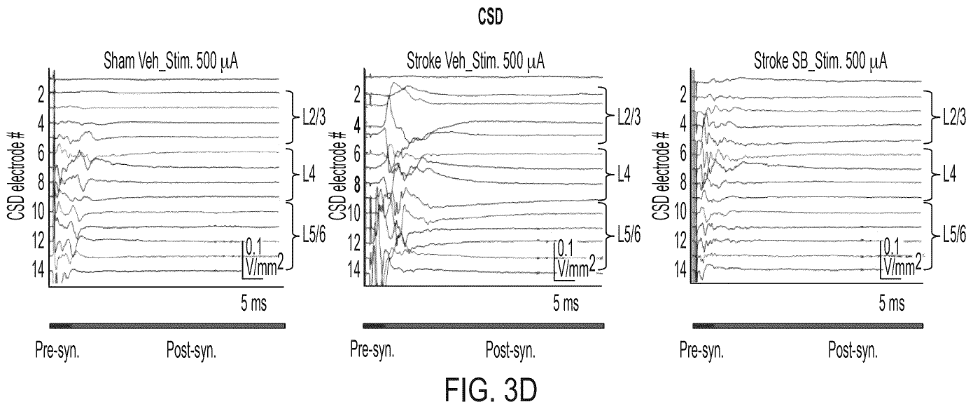

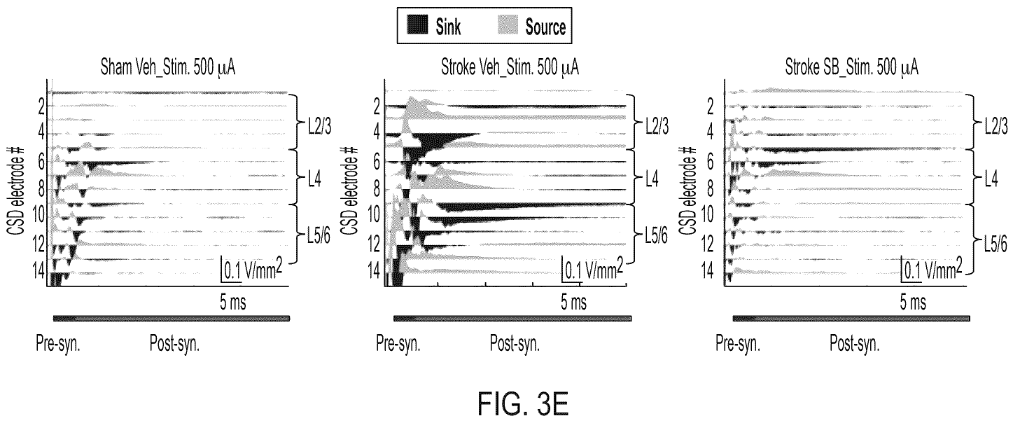

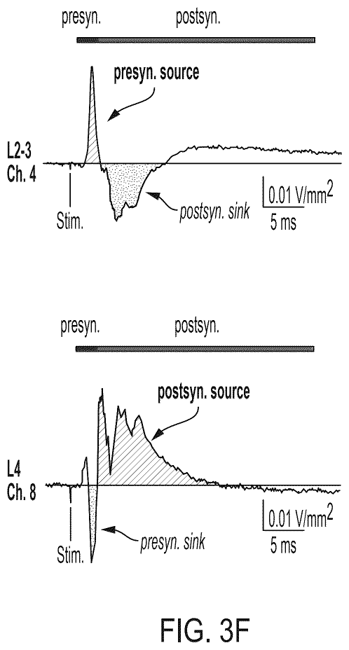

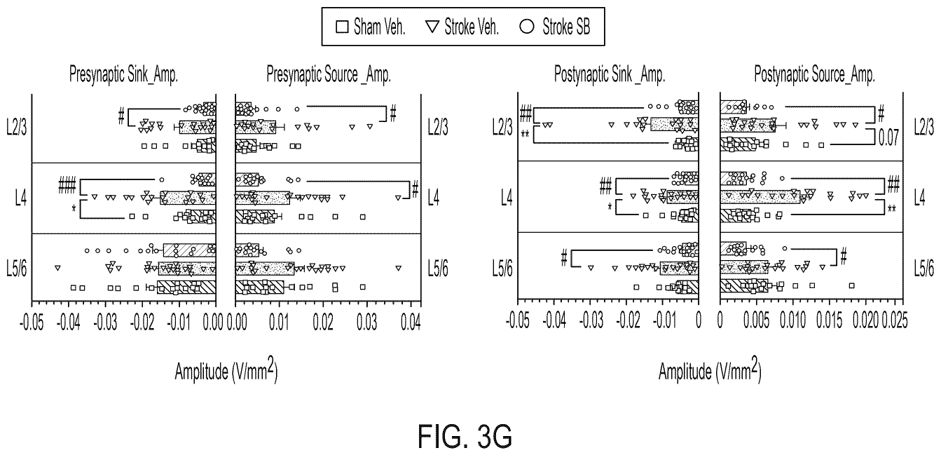

After the adult Sprague Dawley rats were subjected to either stroke or sham surgery, rats were randomly selected to receive either intra-cortical injections of vandefitemcel or a vehicle solution (see FIG. 1). One to two weeks after these injections, acute neocortical coronal slices were prepared and local field potentials (LFPs) were recorded across all cortical laminae in response to increased electrical stimulation of the white matter tract (as seen in FIGS. 3A-3C).

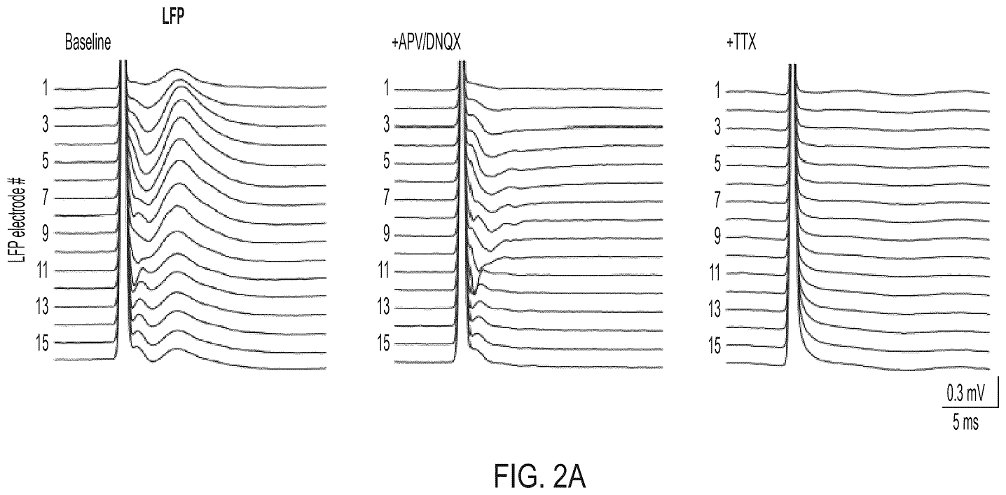

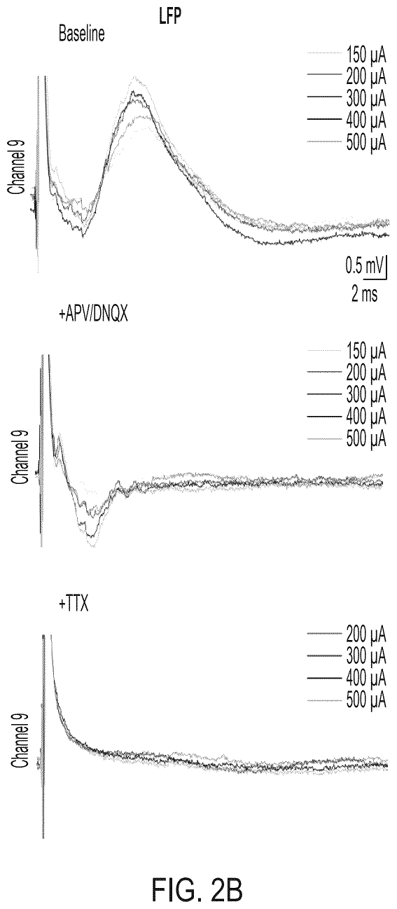

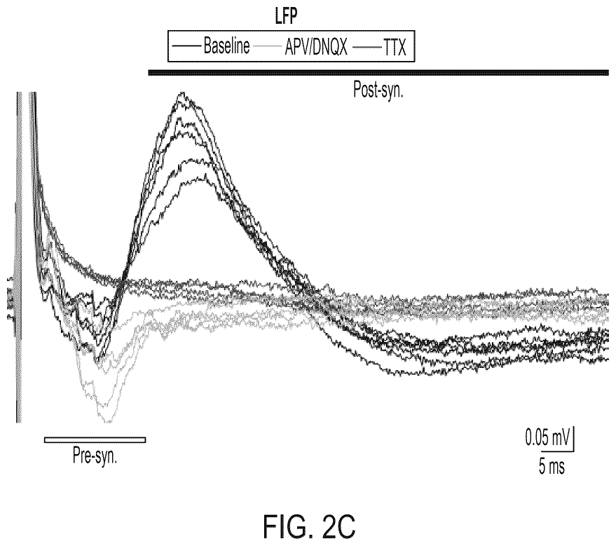

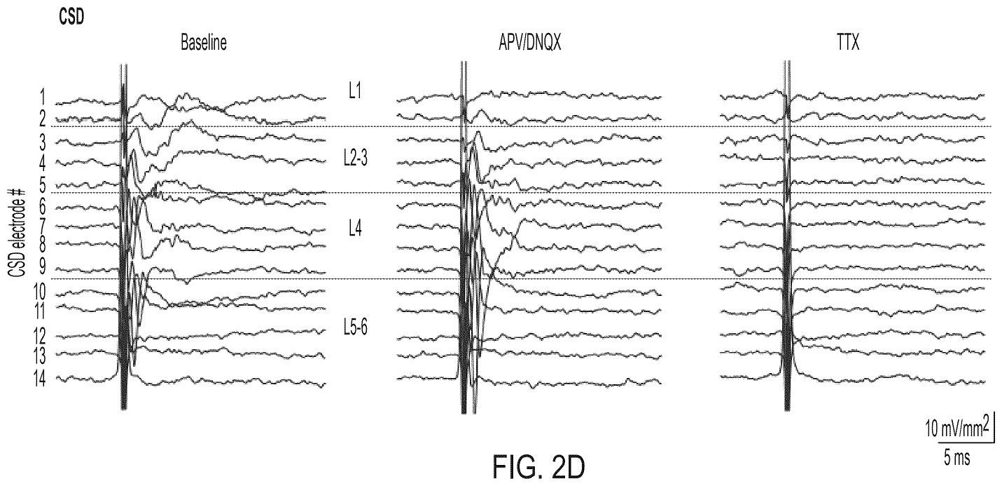

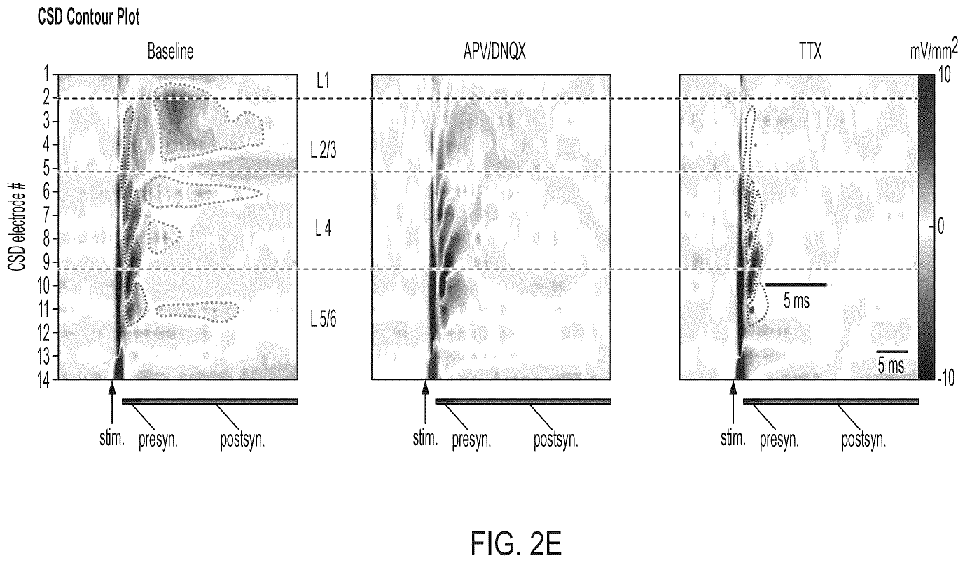

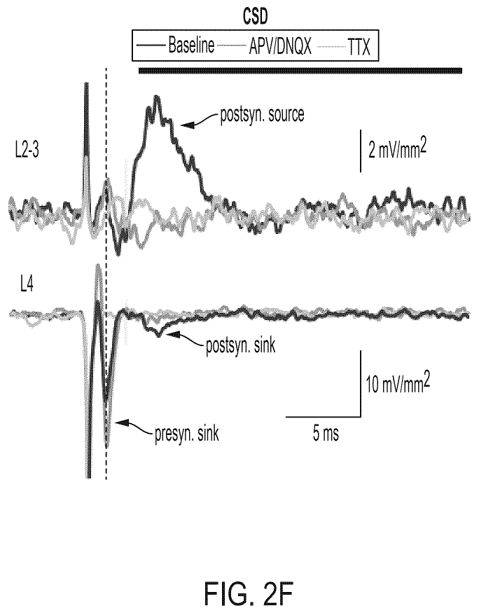

A pharmacological approach was used to identify presynaptic and postsynaptic components of the evoked response: the postsynaptic response was identified by its sensitivity to postsynaptic blockers of glutamatergic neurotransmission APV and DNQX, and the presynaptic response was identified by its sensitivity to the sodium channel blocker TTX (see FIGS. 2A-2F).

FIG. 2A illustrate example local field potential (LFP) recordings in response to electrical stimulation (500 μA) of the white matter from a cortical slice of a Sprague Dawley rat from the stroke vandefitemcel group (treatment group) recorded at baseline (drug-free artificial cerebrospinal fluid (ACSF)), after N-methyl-D-aspartate receptor antagonist (APV) (125 μM) and 6,7-dinitroquinoxaline-2,3-dione (DNQX) application, and in presence of the sodium channel blocker tetrodotoxin (TTX) (1 μM). All three example recordings are from the same slice (n=10 sweeps averaged for each condition).

FIG. 2B are close-ups of LFP recordings evoked in layer 4 at stimuli currents f 150, 200, 300, 400, and 500 μA averaged from 10 sweeps for each stimulus intensity. The use of APV/DNQX (blockers of postsynaptic N-methyl-d-aspartate (NMDA) and alpha-amino-3-hydroxy-5-methyl-4-isoxazole propionic acid (AMPA) glutamatergic receptors) eliminated any postsynaptic response allowing the isolation of the presynaptic response which corresponds to axonal terminal potentials due to the direct electrical stimulation of the presynaptic axons. Increasing the stimulus current intensity in the presence of APV/DNQX produced a larger amplitude of the presynaptic response without any resultant postsynaptic response. After TTX application, the presynaptic response disappeared and only stimulus artifacts remained, and intensifying of stimulus did not change the field recording under TTX perfusion.

FIG. 2C is an overlay of the LFP traces shown in FIG. 2B isolating the presynaptic response (time window between −2 ms and −6 ms after the stimulus onset), and the postsynaptic response (after 6 ms up to 50 ms post-stimulation).

FIG. 2D illustrates current source density (CSD) profiles calculated from field potentials presented in FIG. 2A.

FIG. 2E are CSD contour plots computed from the laminar CSD profiles from FIG. 2D. The interlaminar CSD components as a function of time and cortical depth (relative to the electrode grid). Under the +APV/DNQX perfusion the postsynaptic sources and sinks are eliminated and under the TTX perfusion both presynaptic and postsynaptic sinks and sources are abolished (pre- and postsynaptic CSDs).

FIG. 2F illustrates overlayed traces from CSD electrodes #2 and #9 showing the effects of APV/DNQX and TTX on the presynaptic and postsynaptic source and sink (Baseline CSD is depicted in black, +APV/DNQX in orange and +TTX in gray). All records are from the same preparation and comparable results were observed across preparations (n=4).

FIG. 3A (left) illustrates an experimental design of how the LFP recordings are made ex vivo and show the placement of the recording array spanning all the layers in the peri-stroke cortex and the placement of the stimulation electrode in the white matter. FIG. 3A (right) is a digital photomontage reconstructing the location of the recording electrodes in the coronal NeuN-stained section of the recorded cortex (Si cortex, Bregma −2.5 mm).

FIG. 3B is a coronal NeuN-stained brain section showing the anatomical location of the recording electrode. Electrodes were placed as follows: electrodes 1-2 in layer 1; electrodes 3-5 in layers 2-3; electrodes 6-9 in layer 4; and electrodes 10-14 in layers 5-6.

FIG. 3C illustrates representative LFP responses to a 500 μA stimulation of white matter from representative sham vehicle (Sham Veh), stroke vehicle (Stroke Veh) and stroke vandefitemcel (Stroke SB) conditions.

FIG. 3D illustrates CSD traces derived from the LFP recording in FIG. 3C.

FIG. 3E illustrates responses colored to highlight sinks (darker, defined as a negative deflection) and sources (lighter, defined as a positive deflection) derived from the LFP recording in FIG. 3C.

FIG. 3F are graphs illustrating close-ups on CSDs from layers 2-3 (top) and layer 4 (bottom) from FIG. 3D to illustrate sinks and sources.

FIG. 3G illustrates a distribution of evoked presynaptic and postsynaptic sinks and sources generated from CSDs in layers 2-3, 4, and 5-6 in response to a 500 μA stimulation of white matter. Each dot represents one subject.

To better determine the location, direction, and strength of currents evoked in response to electrical stimulation, a current source density (CSD) analysis (see Aizenman et al., 1996; Pai et al., 2019; Ferguson et al., 2023, Freeman et al., 1975 for discussions of CSD) was performed by calculating the second spatial derivative of the LFP (see FIGS. 3D and 3E). The CSD analysis allows for more localized measures of neuronal activity because the CSD analysis reveals current ‘sinks’ indicating the regions where a net positive current flows into the cells and out of the extracellular space (such as at excitatory synapses), thus creating an extracellular negativity reflected as a downward deflection of CSD (CSD<0). Conversely, current ‘sources’ are regions where the net positive current flows out of the cells back to the extracellular space, thus creating an extracellular positivity reflected as an upward deflection of CSD (CSD>0). To facilitate visualization of CSD profiles along the depth axis (cortical layers), sinks are represented by darker shades in FIG. 3E or stippled patterns in FIG. 3F, and sources are represented by lighter shades in FIG. 3E or cross-hatched patterns in FIG. 3F.

Several processes can be the origin of a current sink or source (Gold et al., 2006; Pettersen et al., 2006; Einevoll et al., 2013). Typically, a current sink in the CSD profile reflects the location and magnitude of synaptic excitation (Steinschneider et al., 1992, Cruikshank et al., 2002). A current source may reflect an outflowing return current resulting from excitatory synaptic input elsewhere on the neuron or may reflect an inhibitory synaptic current (Buzsiki et al., 2012).

As shown in FIG. 3G, stroke had its strongest effect in layer 4, where it increased the sinks and sources of the presynaptic responses relative to the sham control. Also, as shown in FIG. 3G, these metrics were largely normalized in the treatment group that received the vandefitemcel. As seen in FIG. 3G, neither stroke nor vandefitemcel had a significant effect in layers 5-6.

Moreover, FIG. 3G illustrates that stroke had its strongest effect in layer 4, where it increased the sinks and sources of the postsynaptic responses relative to the sham control, and these metrics were largely normalized in the group that had received the vandefitemcel cell transplants. Also, as shown in FIG. 3G, in layers 2-3, stroke increased the postsynaptic sink which was also restored to sham levels in the cell-treated group.

FIG. 4A are graphs showing presynaptic and postsynaptic LFPs in two example channels from layers 2-3 (top graphs) and layer 4 (bottom graphs). The inset graph (rightmost) illustrates an example trace showing the APV/DNQX-sensitive LFP component (postsynaptic), and the TTX-sensitive LFP component (presynaptic).

FIGS. 4B and 4C illustrate the distribution of evoked presynaptic and postsynaptic LFPs in response to a 500 μA stimulation of white matter. Each dot is a result from one subject. For each LFP electrode, the area of the presynaptic LFP and the postsynaptic LFP was calculated.

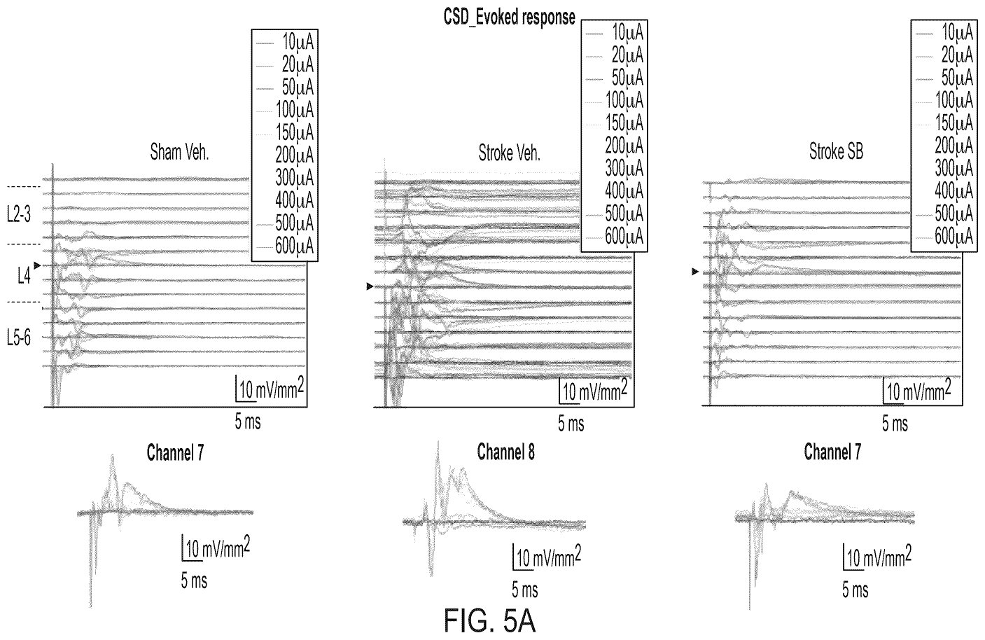

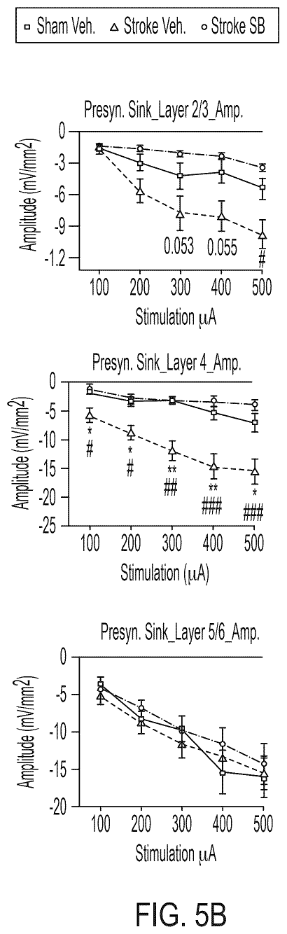

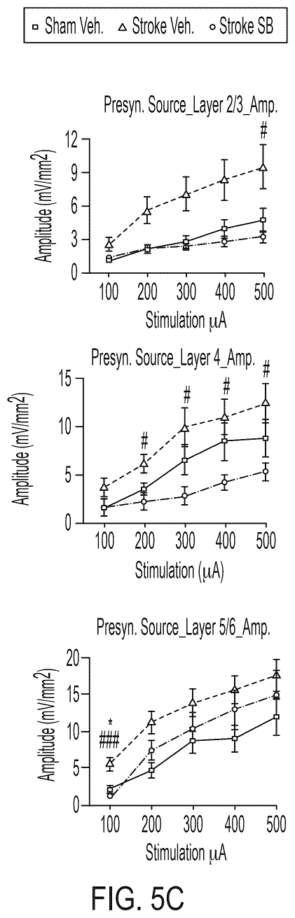

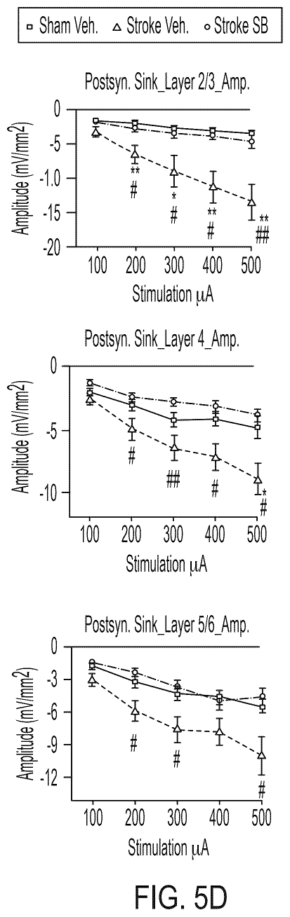

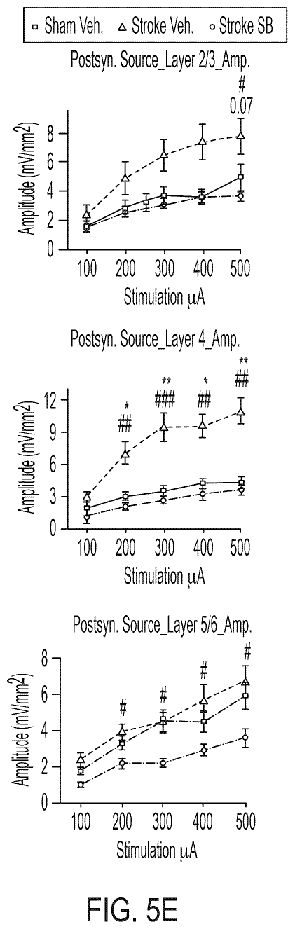

FIG. 5A illustrates CSD traces evoked by white matter stimulation of increasing intensity in representative sham vehicle (Sham Veh), stroke vehicle (Stroke Veh), and stroke vandefitemcel cells (Stroke SB) subjects. Arrows in the top graphs of FIG. 5A indicate the traces expanded in bottom graphs labeled “Channel 7” or “Channel 8.”

FIGS. 5B and 5C are graphs illustrating the amplitude of presynaptic sinks and sources generated from CSDs in layers 2-3, 4, and 5-6 as a function of the intensity of white matter stimulation.

FIGS. 5D and 5E are graphs illustrating the amplitude of postsynaptic sinks and sources generated from CSDs in layers 2-3, 4 and 5-6 as a function of the intensity of white matter stimulation.

Also measured was the evolution of LFPs and CSDs in response to increasing stimulation intensity of white matter. FIGS. 5B and 5C show that stroke had the largest effect on presynaptic sinks in layer 4 where it increased the strength of the CSDs at most stimulation intensities, and these effects were all normalized by cell treatment.

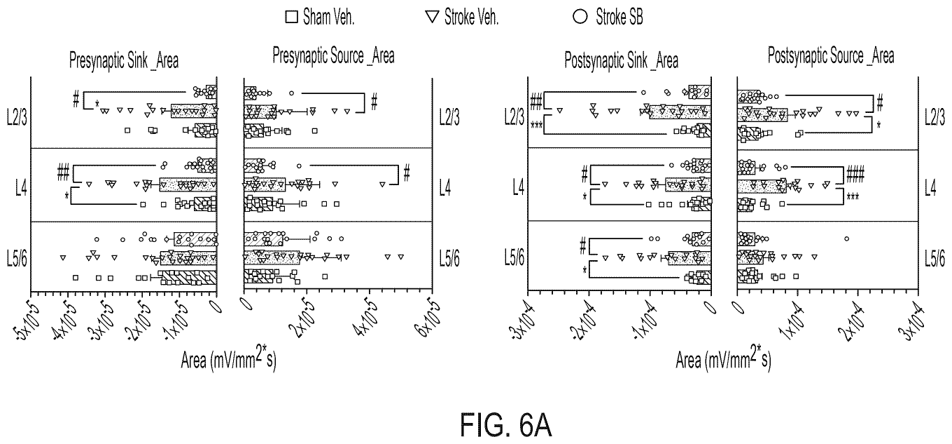

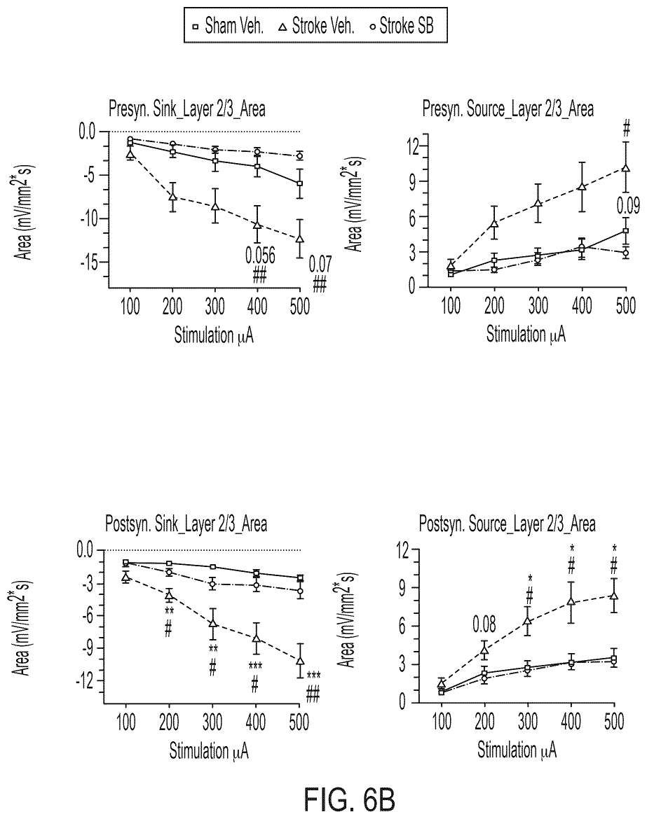

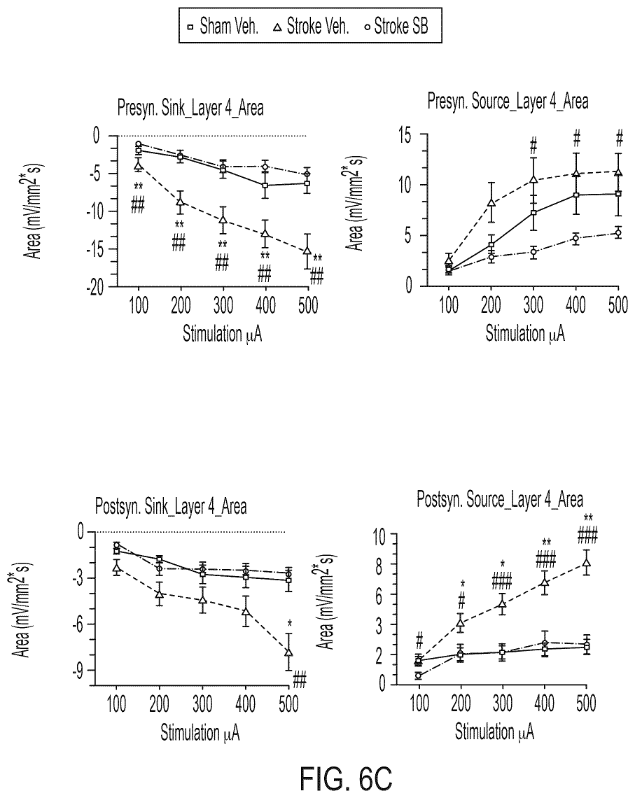

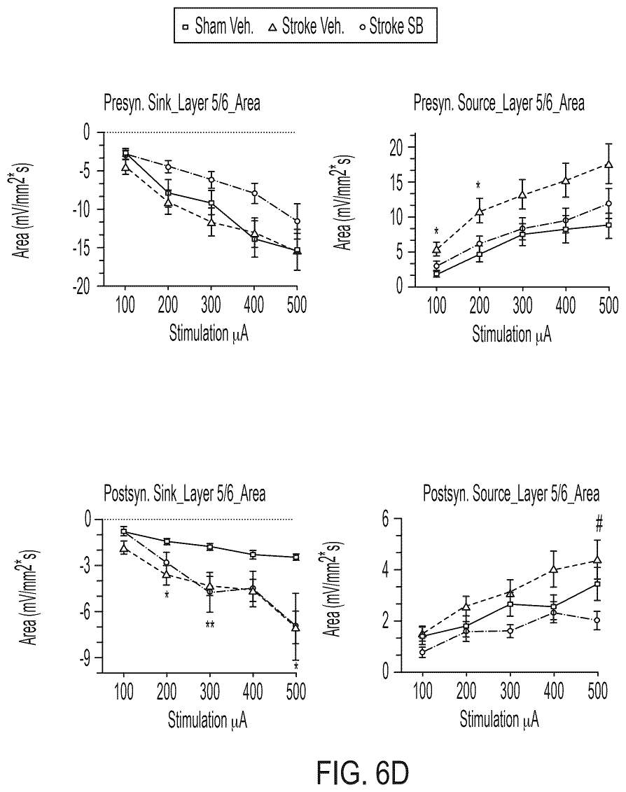

In response to increasing stimulation intensity of white matter, stroke had the largest effect on postsynaptic sinks in layers 2-3 and postsynaptic source in layer 4, where it consistently increased CSDs at various intensities. These effects were all normalized by cell treatment, as seen in FIGS. 5D and 5E and FIGS. 6A-6D.

FIG. 6A are graphs illustrating a distribution of evoked presynaptic and postsynaptic sinks and sources generated from CSDs in layers 2-3, 4, and 5-6 in response to a 500 μA stimulation of white matter. Each dot is a result of one subject.

FIG. 6B are graphs illustrating the area of the presynaptic and postsynaptic sinks and sources generated from CSDs in layers 2-3 as a function of the intensity of white matter stimulation from the channels.

FIG. 6C are graphs illustrating the area of the presynaptic and postsynaptic sinks and sources generated from CSDs in layer 4 as a function of the intensity of white matter stimulation from the channels.

FIG. 6D are graphs illustrating the area of the presynaptic and postsynaptic sinks and sources generated from CSDs in layers 5-6 as a function of the intensity of white matter stimulation from the channels.

Accordingly, the LFP/CSD analysis provides evidence that stroke results in cortical circuit hyperexcitability, which appears to be the most pronounced in cortical layer 4, and that vandefitemcel transplantation restores normal cortical network excitability.

### Survival of Vandefitemcel Post-Transplantation

To investigate which cellular and molecular changes can explain the observed effect of vandefitemcel cell treatment, the brains of a separate cohort were harvested on Day 7 after treatment, which was the earliest time point at which a clear amelioration of post-stroke cortical hyperexcitability by the transplant in previous electrophysiological experiments was observed.

The first step for the histology analysis was to confirm the successful delivery of the vandefitemcel cells.

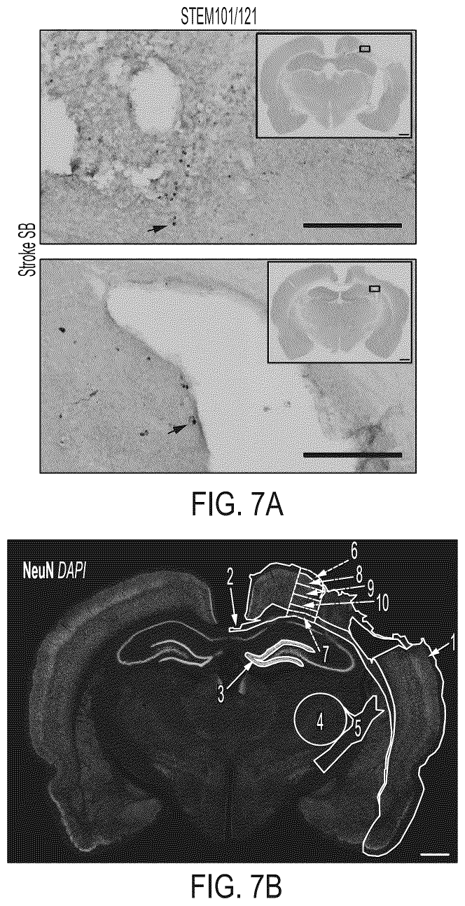

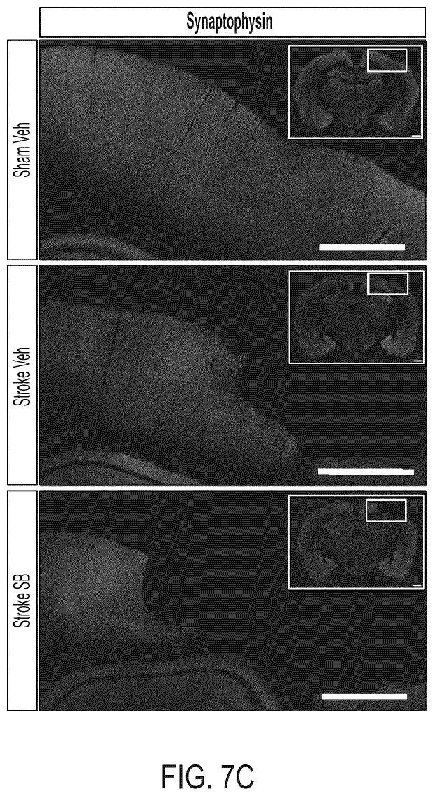

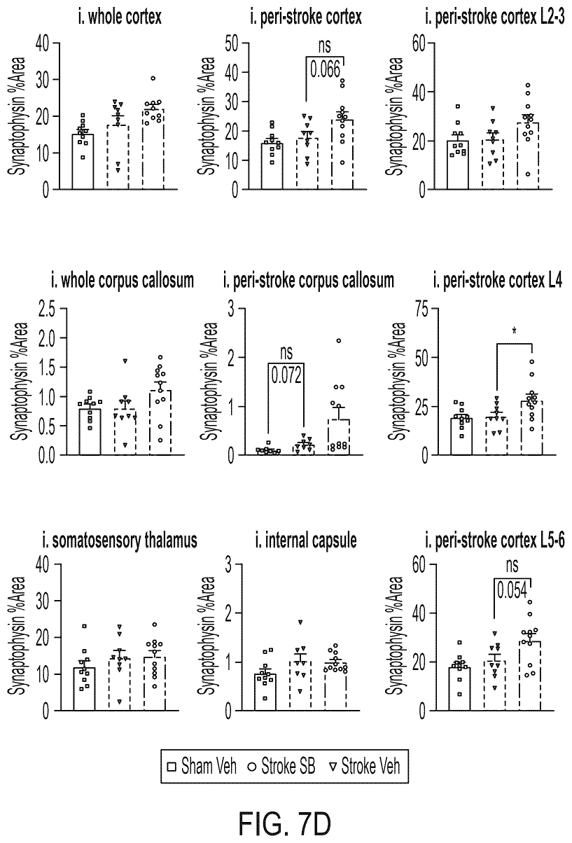

FIG. 7A illustrates detection of vandefitemcel cells (human STEM101/121+; arrowheads) in cortical (top image) and subcortical (bottom image) locations one week after injection into the peri-stroke cortex (treatment was administered one month post-stroke).

Analysis of staining for STEM101 and STEM121, two markers of human cells, showed the presence of 332±282 total vandefitemcel cells per brain (mean±SD) seven days after administration into the peri-stroke cortex. This corresponds to 0.18±0.16% (mean±SD) surviving vandefitemcel cells relative to the originally injected dose of 1.8×10\hat{ }⁵ cells. STEM101/121⁺ vandefitemcel cells were located not only in the cortex, but also in subcortical areas, and in one case even in the corpus callosum of the contra-lesional hemisphere. These results show that vandefitemcel cells exert their beneficial effects even if the majority of the transplanted cells do not survive longer than a few days after transplantation.

### Vandefitemcel's Effect on Brain Volume

The rat brain cells were then observed to determine which changes in the brain tissue are connected to the observed changes in cortical excitability after vandefitemcel treatment.

FIG. 7B illustrates an annotated coronal brain section of a rat five weeks post-stroke indicating the ipsilesional regions of interest (ROIs) analyzed using immunofluorescence histology stains. The illustration comprises the following labels: lighter shading is NeuN, darker shading is DAPI, 1 is the whole cortex, 2 is the whole corpus callosum, 3 is the hippocampal dentate gyrus, 4 is the somatosensory thalamus, 5 is the internal capsule, 6 is the peri-stroke cortex, 7 is the peri-stroke corpus callosum, 8 is the peri-stroke cortex layers 2-3, 9 is the peri-stroke cortex layers 4, and 10 is the peri-stroke cortex layers 5-6.

For these analyses, the ipsilesional brain hemisphere was broken down into nine regions of interest (ROIs) (see FIG. 7B), focusing on the peri-stroke area, but also including other, functionally connected brain regions, to detect potential remote effects.

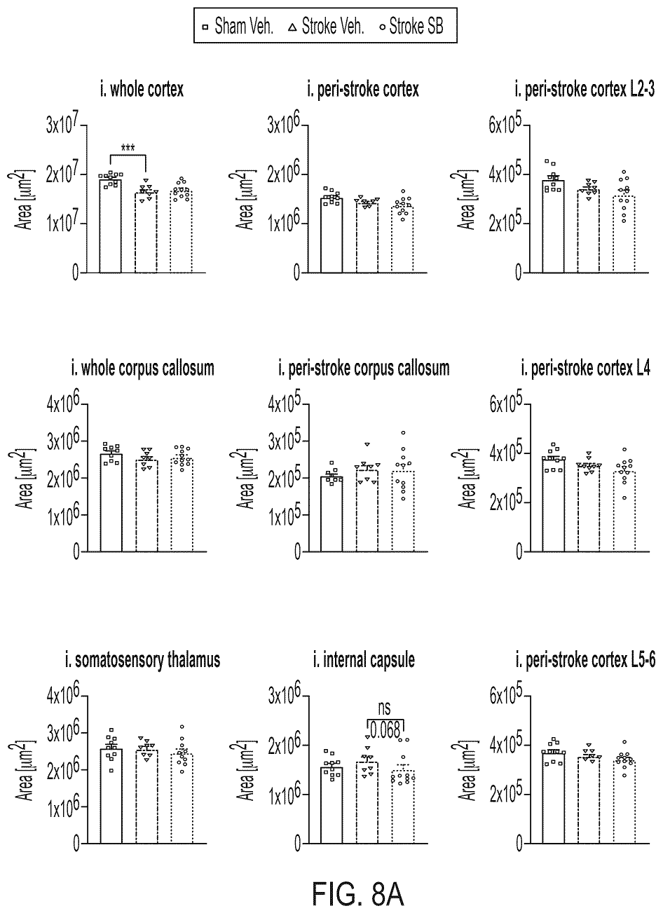

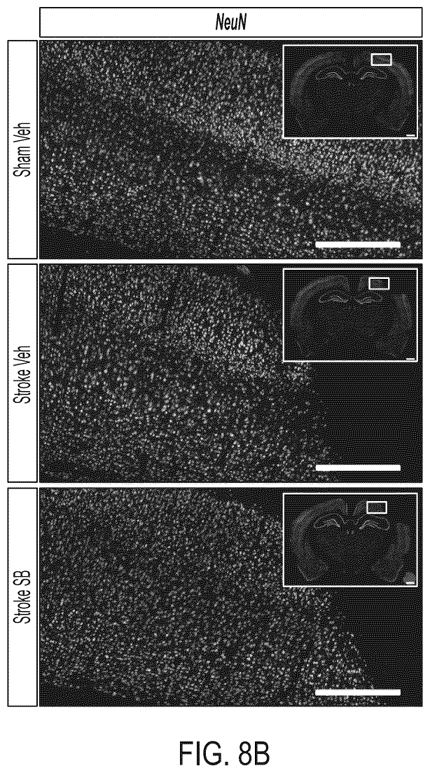

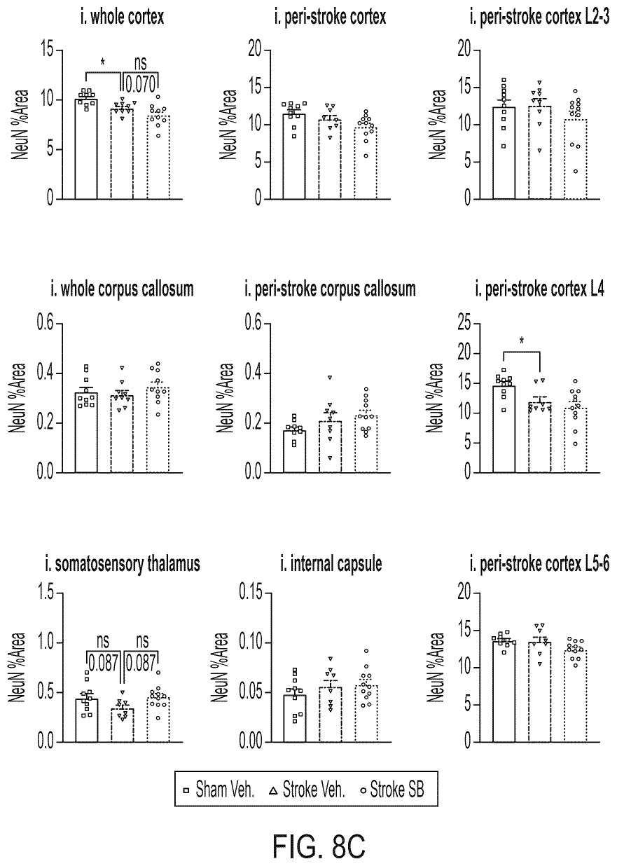

FIG. 8A shows various graphs of size measurements of analyzed brain regions showing a significant shrinkage of the ipsilesional cortex in the stroke vehicle group in comparison to the sham vehicle group as a result of stroke.

FIG. 8B are images representing staining of NeuN⁺ neurons in the peri-stroke region.

FIG. 8C are diagrams showing percentages of each ROI's area stained for NeuN in the sham vehicle (Sham Veh, squares), stroke vehicle (Stroke Veh, triangles), and stroke vandefitemcel (Stroke SB, circles) groups. Stroke significantly decreased the percentage area of NeuN staining in the ipsilesional cortex, and also in layer 4 of the peri-stroke cortex.

As expected, stroke caused a significant loss of volume and neurons (NeuN⁺ cells) in the ipsilesional cortex. The decrease in NeuN signal was also significant in cortical layer 4 of the peri-stroke region, and a similar trend (p=0.087) was observed in the ipsilesional somatosensory thalamus. Vandefitemcel treatment did not significantly affect the volume of the analyzed brain regions or NeuN area in comparison to the stroke vehicle group. There were trends for a decrease of the volume of the ipsilesional internal capsule (p=0.068), a decrease in NeuN area in the ipsilesional cortex (p=0.070), and an NeuN area increase in the ipsilesional somatosensory thalamus (p=0.087) (see FIGS. 8A-8C).

### Vandefitemcel Transplantation Modulates Synapses and Increases the Amount of the GABA Reuptake Transporter GAT-1

Since the electrophysiological experiments showed that both presynaptic and postsynaptic neuronal signaling was increased after stroke and normalized by vandefitemcel treatment, a histological investigation was undertaken that focused on synaptic vesicles, which are at the center of neurotransmission, and whose numbers are influenced by presynaptic signaling and in turn change postsynaptic excitability. Rat brain sections were first stained for synaptophysin, an integral membrane protein of presynaptic vesicles (see FIG. 7C for images representing synaptophysin (synaptic vesicle marker) staining in the peri-stroke region). To quantify synaptophysin staining, the area covered by synaptophysin signal was measured and expressed as a percentage of the ROI's area.

FIG. 7D are diagrams showing percentages of each ROI's area stained for synaptophysin in the sham vehicle (Sham Veh, squares), stroke vehicle (Stroke Veh, triangles), and stroke vandefitemcel (Stroke SB, circles) groups. Synaptophysin staining in each ROI showed a significant increase in layer 4 of the peri-stroke cortex in the stroke vandefitemcel group in comparison to vehicle treatment (with similar trends in the peri-stroke cortex overall, as well as in layers 5-6).

No significant effects of stroke on synaptophysin staining was found from these measurements. There was only a trend (p=0.072) for an increase of synaptophysin in the peri-stroke corpus callosum in the stroke vehicle group compared to the sham group. Therefore, presynaptic and postsynaptic hyperexcitability observed after the PT stroke does not appear to result from increased synaptic vesicle formation.

By contrast, vandefitemcel cell treatment/transplantation induced a significant increase of synaptophysin in layer 4 of the peri-stroke S1 cortex relative to vehicle treatment. Similar trends were found in the peri-stroke cortex overall (p=0.066), as well as in layers 5-6 (p=0.054) (see FIG. 7D). This increase in synaptophysin⁺ synaptic vesicles suggests that vandefitemcel administration/transplantation modulates synaptic activity either by stimulating the formation of additional synapses (synaptogenesis) or by increasing the signaling strength of existing synaptic connections (synaptic plasticity). Others have observed induction of synaptic plasticity after treatment with MSCs in a vascular dementia model (Wang et al., 2019), and after treatment with exosomes derived from rat BM-MSCs in the acute phase after ischemic stroke in a rat middle cerebral artery occlusion (MCAO) model (Xin et al., 2013). The increase in synaptophysin⁺ synaptic vesicles in layer 4 (where a strong effect of stroke and vandefitemcel cells on neuronal excitability was detected) suggests that modulation of synaptic activity is partly responsible for the vandefitemcel-induced changes in cortical excitability. The next step was to try to narrow down the neurotransmitters contained in the synaptic vesicles that were increased by vandefitemcel treatment.

A possible cause of stroke-induced cortical hyperexcitability could be a decrease in inhibitory, GABAergic signaling, and it was hypothesized that an increase in GABAergic signaling might explain the normalizing effect of vandefitemcel treatment on cortical excitability. Rat brain sections were stained for VGAT, the vesicular GABA transporter.

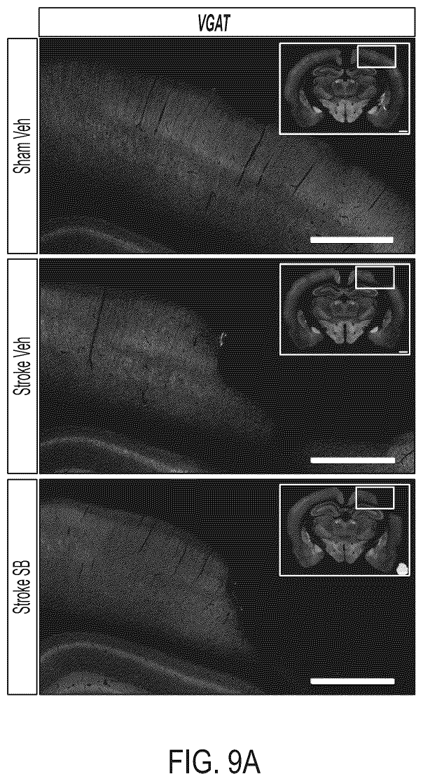

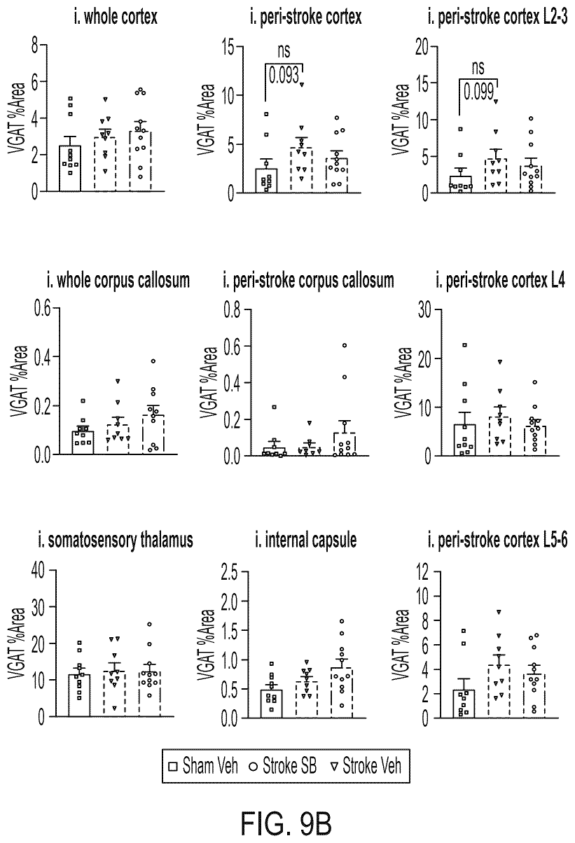

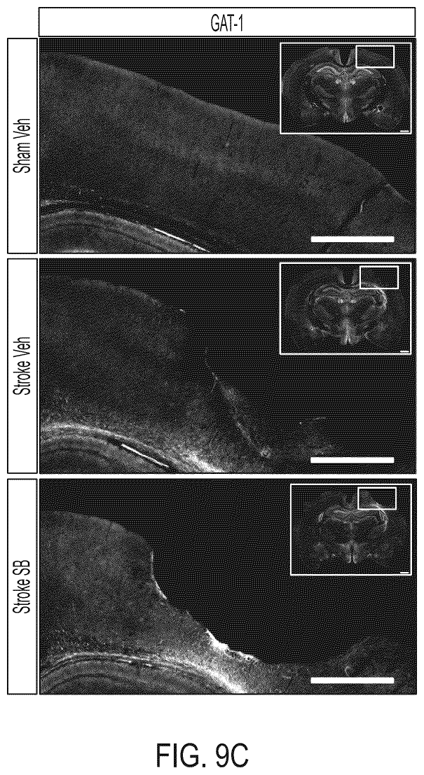

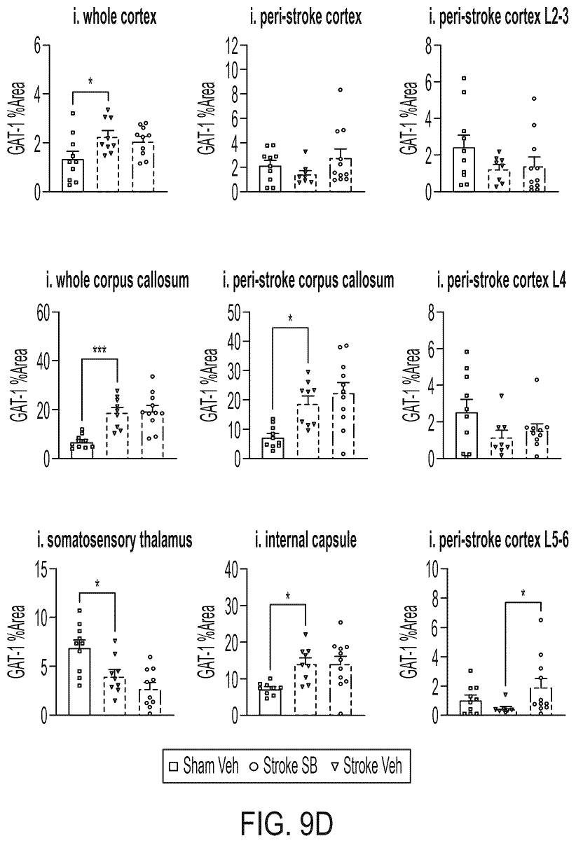

FIG. 9A are images representing vesicular GABA transporter (VGAT) staining in the peri-stroke cortex.

FIG. 9B illustrates the results of the staining illustrated in FIG. 9A, by the percentage of area occupied by VGAT staining for each ROI in the sham vehicle (Sham Veh, squares), stroke vehicle (Stroke Veh, triangles), and stroke vandefitemcel (Stroke SB, circles) groups.

An analysis of the VGAT⁺ signal did not show significant changes induced by stroke, only slight trends for an increase in VGAT⁺ area in the peri-stroke cortex (p=0.093) and in this region also specifically in layer 2-3 (p=0.099). Vandefitemcel cell treatment/transplantation did not have any significant effects on the expression of VGAT (see FIGS. 9A and 9B).

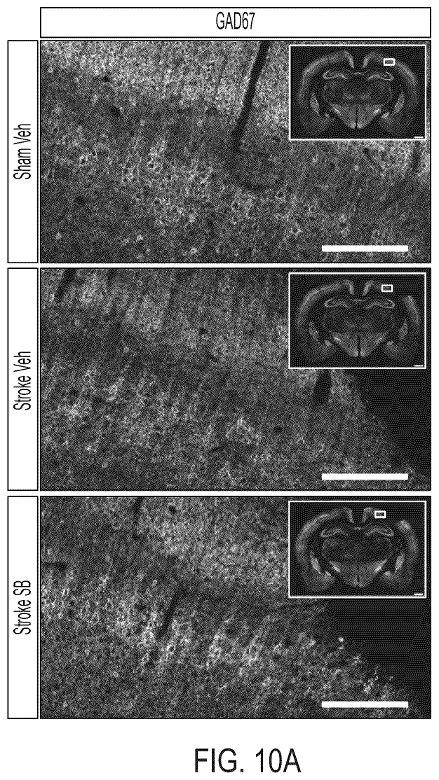

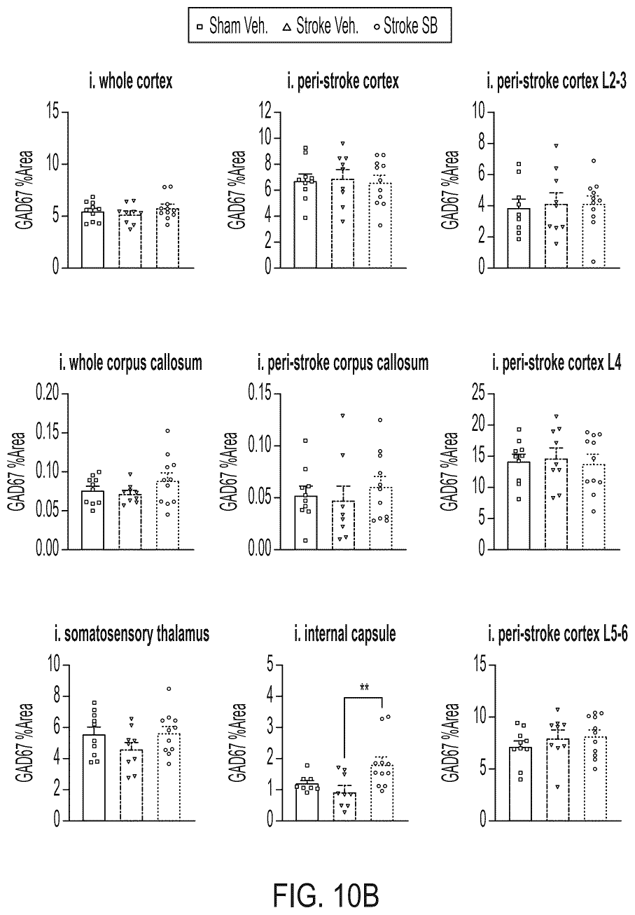

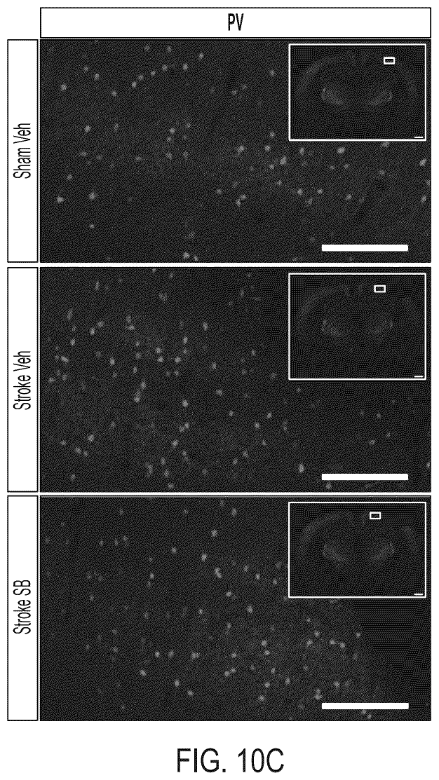

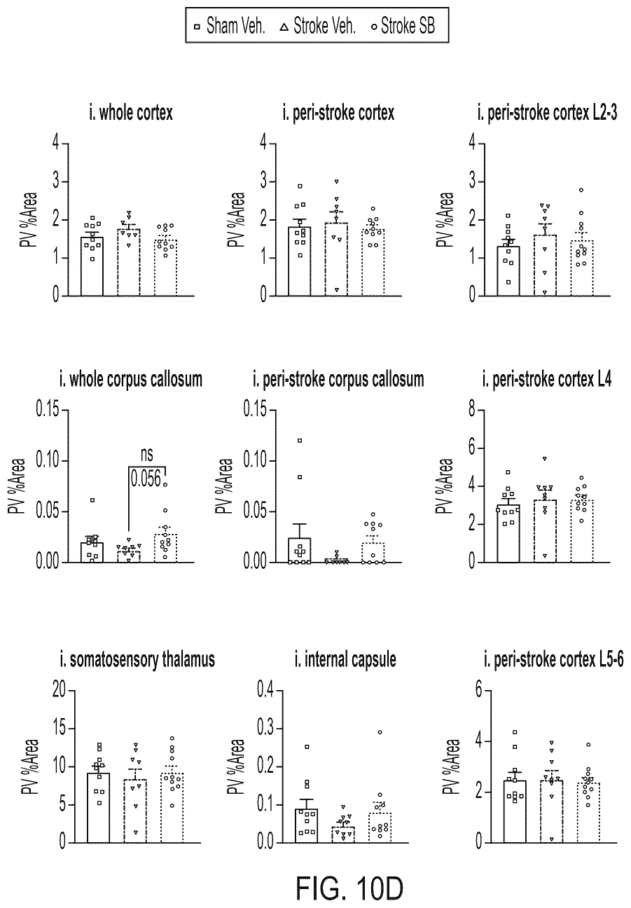

To further analyze the effects of stroke and vandefitemcel cell treatment/transplantation on GABAergic neurons such as glutamic acid decarboxylase 67 (GAD67), rat brains were stained for a marker for this class of neurons. FIG. 10A are images representing GAD67 staining in the peri-stroke region.

FIG. 10B illustrates the results of the staining illustrated in FIG. 10A, by the percentage of each ROI's area stained for GAD67 in the sham vehicle (Sham Veh, squares), stroke vehicle (Stroke Veh, triangles) and stroke vandefitemcel (Stroke SB, circles) groups.

Analysis of the GAD67 area showed no significant effects of stroke, when compared to the sham group. Compared to the stroke vehicle group, vandefitemcel cell treatment/transplantation significantly increased GAD67 signal only in the ipsilesional internal capsule (a region in which GAD67 expression is generally low, and where this change might have limited functional impact) (see FIG. 10B).

FIG. 10C are images representing stains for parvalbumin-positive (PV+) GABAergic interneurons in the peri-stroke cortex.

FIG. 10D illustrates the results of the staining illustrated in FIG. 10C, showing that both stroke and vandefitemcel treatment did not significantly change the percentage area of PV+ staining in any of the analyzed ROIs.

Similarly, no changes in the number of PV⁺ GABAergic interneurons were detected (see FIG. 10C) by either stroke or vandefitemcel cell treatment/transplantation. Only in the ipsilesional corpus callosum, where PV expression is very low and where few, if any, PV⁺ interneurons should be located, was there a trend (p=0.056) for vandefitemcel cell treatment/transplantation to increase PV⁺ area in comparison to the stroke vehicle group (see FIG. 10D).

Since neither changes in the presence of GABAergic synaptic vesicles nor of GAD67⁺ or PV⁺ GABAergic neurons seemed responsible for the stroke-induced or vandefitemcel-induced changes in cortical excitability, the focus was shifted to transporters that regulate the availability of GABA. GABA reuptake transporters, such as GAT-1, remove GABA from the synaptic gap and are located in neurons and glia. An analysis of GAT-1 (see FIG. 9C) showed that stroke significantly increased the GAT-1+ area in the ipsilesional cortex and corpus callosum (here also specifically in the peri-stroke region), and the internal capsule, whereas a significant decrease was observed in the ipsilesional somatosensory thalamus (compared to the sham vehicle group). FIG. 9C are images representing stains for GABA transporter 1 (GAT-1) in the peri-stroke cortex.

While vandefitemcel cell treatment/transplantation did not affect these stroke-induced effects on GAT-1 expression, vandefitemcel cells did significantly increase GAT-1 in the peri-stroke cortex in layers 5-6 (see FIG. 9D).

FIG. 9D illustrates the results of the staining illustrated in FIG. 9C, by the percentage of area occupied by GAT-1 staining for each ROI. In this example, a significant stroke-induced increase was observed in the ipsilesional cortex, corpus callosum (also specifically in the peri-stroke region), and the internal capsule, whereas a significant decrease was observed in the somatosensory thalamus (in the comparison of stroke vehicle vs. sham vehicle).

GABA transporters play important roles in regulating neuronal network excitability by maintaining a balance of available GABA not only in the synaptic cleft but also for extrasynaptic, tonic GABA signaling (Bhatt et al., 2023). Thus, the vandefitemcel transplantation/treatment-mediated increase of GAT-1 in the peri-stroke cortical layer 5-6 observed here may change the availability of GABA in the extracellular milieu, which might play a part in the observed modulation of cortical excitability by vandefitemcel treatment. Similarly, a study in another PT ischemic stroke model found that an increase of GAT-1 in the peri-infarct cortex has a positive impact on recovery after stroke (Lin et al., 2021).

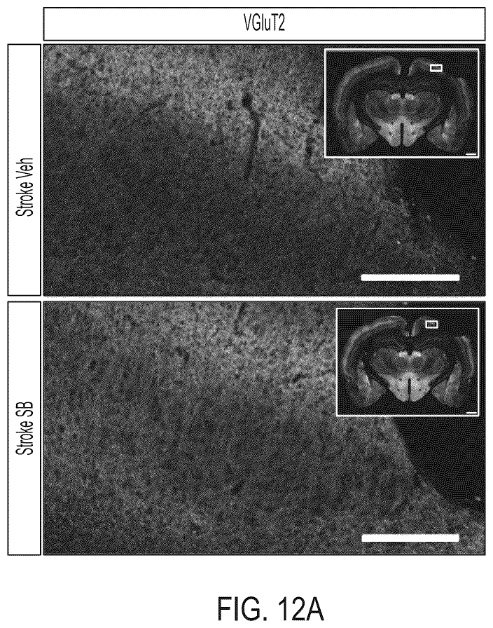

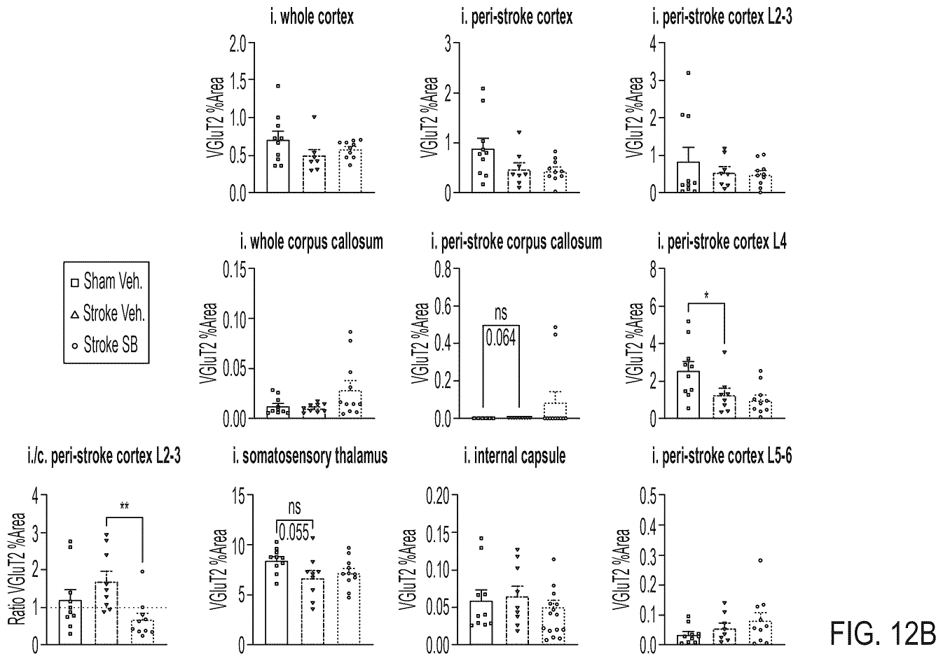

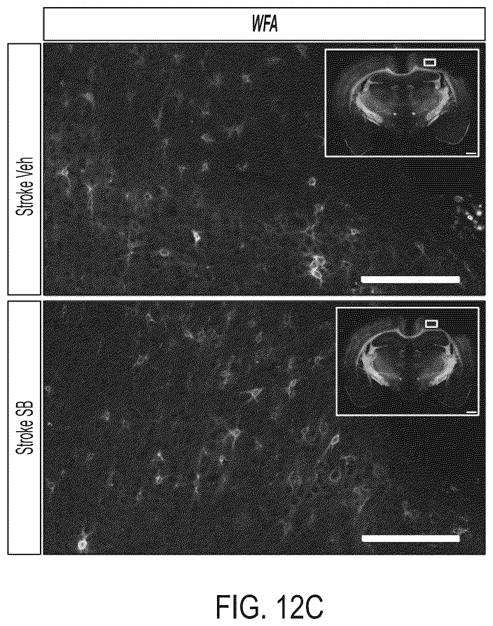

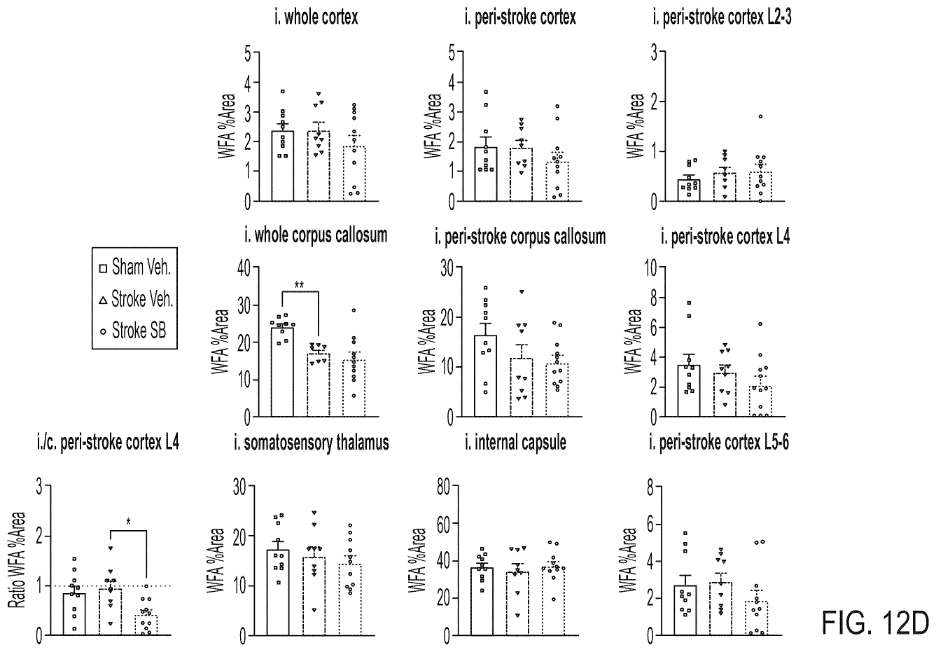

Increased circuit excitability after a stroke could also reflect an increase in excitatory, glutamatergic signaling. To test this hypothesis, rat brains were stained for VGluT2, a marker of glutamatergic vesicles (see FIG. 12A for images representing VGluT2 staining in the peri-stroke cortex). Compared to the sham vehicle group, stroke significantly decreased VGluT2 signal in the peri-stroke cortex in layer 4. There was also a trend (p=0.055) for a stroke-induced decrease in VGlutT2+ area in the ipsilesional somatosensory thalamus, and a trend (p=0.064) for increased VGlutT2 in the peri-stroke corpus callosum (where hardly any VGlutT2 is expressed). Vandefitemcel cell treatment/transplantation did not have any significant effects on VGluT2⁺ area compared to the stroke vehicle group. However, an analysis of the ratio of VGluT2 expression between the corresponding regions of the ipsilesional and contralesional hemispheres showed a significantly lower ratio after vandefitemcel cell treatment/transplantation compared to vehicle treatment in the peri-stroke cortex in layer 2-3 (see FIG. 12B).

FIG. 12B illustrates the results of the staining illustrated in FIG. 12A, by the percentage of each ROI's area stained for VGluT2 in the sham vehicle (Sham Veh, squares), stroke vehicle (Stroke Veh, triangles) and stroke vandefitemcel (Stroke SB, circles) groups. Stroke significantly lowered the percentage area of VGluT2⁺ glutamatergic synaptic vesicles in layer 4 of the peri-stroke cortex, with a similar trend (p=0.055) in the ipsilesional somatosensory thalamus, and a trend (p=0.064) for VGlutT2 increase in the peri-stroke corpus callosum (where VGlutT2 levels are very low).

In an in vitro experiment, neither MSCs nor vandefitemcel cells affected the presence of VGluT⁺ synaptic vesicles in rat embryonic neural cells (Aizman et al., 2014), which is consistent with observations that vandefitemcel do not alter VGluT2 expression. The stroke-induced reduction of the VGluT2 signal in the peri-stroke cortex was surprising. Based on these findings, one conclusion that can be made is that stroke-induced cortical hyperexcitability is not caused by an increase in glutamatergic signaling. The lower VGluT2 ratio between the ipsilesional and contralesional peri-stroke region after vandefitemcel treatment indicates a reduction of ipsilesional glutamatergic synaptic vesicles that contributes to the normalization of cortical excitability.

Neuronal excitability and post-stroke recovery are also influenced by perineuronal nets (PNNs), extracellular matrix structures preferentially surrounding inhibitory PV+ interneurons that are thought to stabilize synaptic connections (see for example, Quattromani et al., 2018; Dzyubenko et al., 2023). Rat brain sections were stained with Wisteria floribunda agglutinin (WFA), which binds to PNNs (see FIG. 12C for images representing staining of wisteria floribunda agglutinin (WFA⁺) perineuronal nets (PNNs) in the peri-stroke region). A significant reduction in WFA⁺ signal in the stroke compared to the sham group was discovered, but only in the ipsilesional corpus callosum, a region which should only contain few PNNs and neurons, so the functional relevance of this finding is not clear. Vandefitemcel cell treatment/transplantation did not affect WFA⁺ PNN area. However, an analysis of the ratio of WFA⁺ PNN staining between the ipsilesional and contralesional hemispheres showed a significantly lower ratio after vandefitemcel cell treatment/transplantation in the peri-stroke cortex in layer 4 (see FIG. 12D), where the ratio of the mean size of WFA⁺ PNNs was also significantly reduced. These results indicate subtle changes in PNN structure after vandefitemcel treatment.

FIG. 12D illustrates the results of the staining illustrated in FIG. 12C, via an analysis of the percentage of area occupied by WFA staining which labels PNNs in each ROI. The WFA⁺ PNN percentage area was significantly lower in the ipsilesional corpus callosum in the stroke vehicle group compared to the sham vehicle group. Vandefitemcel treatment did not change the WFA⁺ PNN percentage area. An analysis of the ipsilesional:contralesional ratio of the WFA⁺ PNN percentage area showed a significantly lower ratio in layer 4 of the peri-stroke in the stroke vandefitemcel group in comparison to the stroke vehicle group.

A transient loosening of PNNs in the post-stroke motor cortex has been observed in the subacute phase after stroke in an MCAO model and has been linked to post-stroke synaptic remodeling and neuroplasticity facilitating motor recovery (Dzyubenko et al., 2023). The subtle vandefitemcel-induced changes in PNNs in the peri-stroke layer 4 cortex that were detected by analyzing the ipsilesional vs. contralesional ratio might indicate that while this treatment did not affect the overall presence of PV⁺ interneurons, their signaling properties may be altered, which might contribute to the restoration of cortical excitability after vandefitemcel treatment.

To summarize, the causes for chronic cortical hyperexcitability one month after stroke appear to be multifactorial and complex. Stroke led to a significant loss of glutamatergic synaptic vesicles in the peri-stroke cortex. Stroke did not affect GAD67⁺ GABAergic neurons, PV⁺ GABAergic interneurons, or PNNs in the peri-stroke cortex, but caused a marked change in expression of the GABA transporter GAT-1 in different brain regions.

Vandefitemcel treatment seems to exert its normalizing effects on cortical hyperexcitability by modulating synaptic activity (as indicated by the increase in synaptophysin staining; though not specifically in GABAergic synaptic vesicles), and by increasing GAT-1 in the peri-stroke cortex. Additionally, even though vandefitemcel treatment did not rescue the stroke-induced loss of glutamatergic vesicles in the peri-stroke cortex, a comparison between the ipsilesional and contralesional hemispheres indicated a relative reduction of glutamatergic synaptic vesicles in the peri-injury region, and a similar analysis showed changes in the level of PNNs in the peri-stroke cortex which both might contribute to the modulation of cortical excitability.

### Vandefitemcel Transplantation Increases the Endogenous Production of BDNF

The previously discussed experimental results revealed that ischemic strokes led to chronic cortical network hyperexcitability. For example, ischemic stroke caused a loss of brain tissue, neurons, and myelin. It was hypothesized that these structural changes contribute to cortical hyperexcitability as well as stroke-induced reduction of the growth factor brain-derived neurotrophic factor (BDNF).

Previous students have shown that reduced levels of BDNF can drive hyperexcitability by reducing GABA inhibition in other brain regions such as the hippocampus (Tanaka et al., 1997; Frerking et al., 1998) and amygdala (Meis et al., 2019).

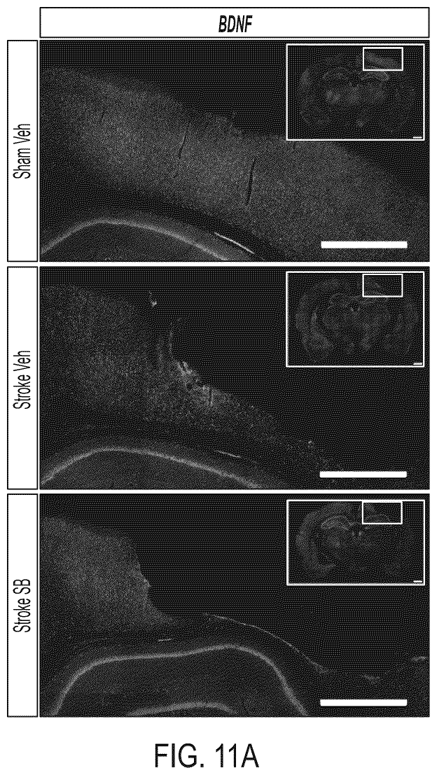

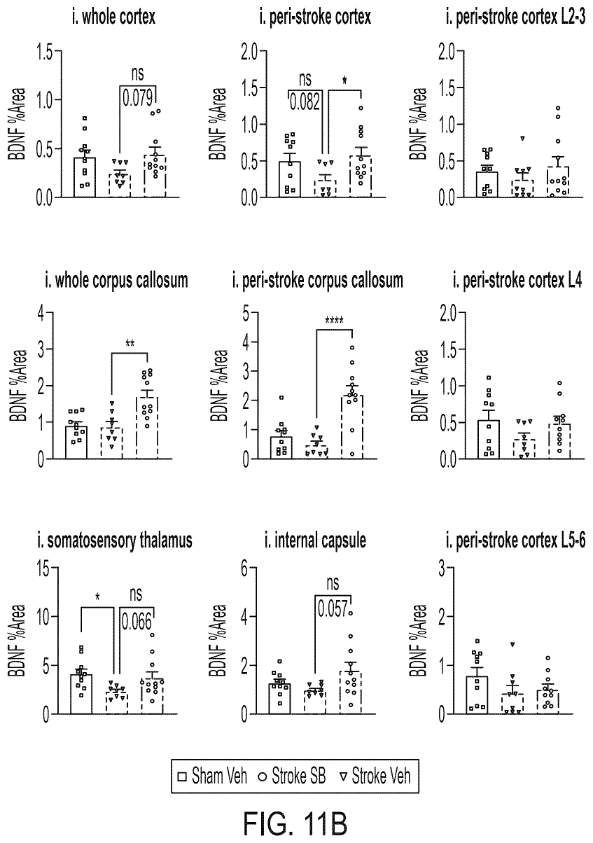

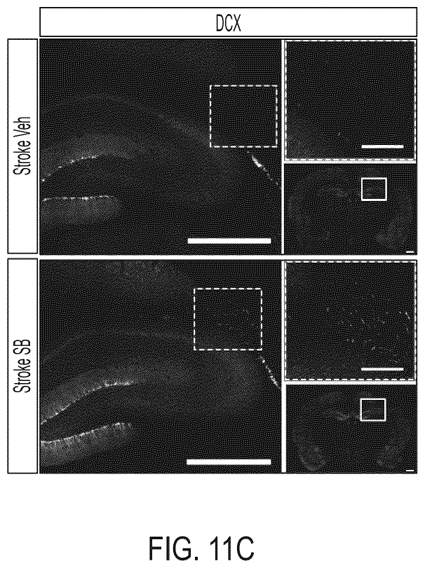

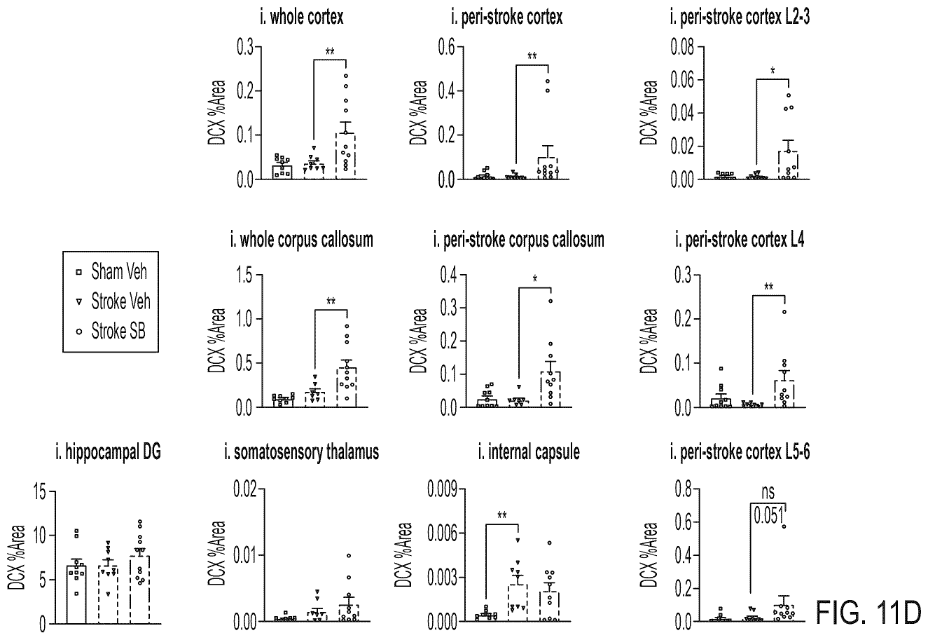

BDNF is also known to stimulate GAT-1-mediated GABA transport (Vaz et al., 2011). As a result, rat brain sections were also stained for BDNF (see FIG. 11A for images representing BDNF staining in the peri-stroke region).

FIG. 11B illustrates the results of the staining illustrated in FIG. 11A, by the percentage of each ROI's area stained for BDNF in the sham vehicle (Sham Veh, squares), stroke vehicle (Stroke Veh, triangles), and stroke vandefitemcel (Stroke SB, circles) groups. Analysis of the percentage area of BDNF staining showed a significant stroke-induced BDNF decrease in the ipsilesional somatosensory thalamus, and a similar trend (p=0.082) in the peri-stroke cortex. Vandefitemcel treatment significantly increased BDNF in the ipsilesional corpus callosum, and the peri-stroke region of both the cortex and the corpus callosum.

It was discovered that stroke significantly decreased BDNF signal in the ipsilesional somatosensory thalamus, and to a less significant extent (p=0.082) in the peri-stroke cortex in comparison to the Sham Veh group. Vandefitemcel cell treatment/transplantation significantly increased BDNF in the ipsilesional corpus callosum and the peri-stroke region of both the cortex and the corpus callosum. Similar trends were observed in the ipsilesional hemisphere in the cortex (p=0.079), the somatosensory thalamus (p=0.066), and the internal capsule (p=0.057) (see FIG. 11B).

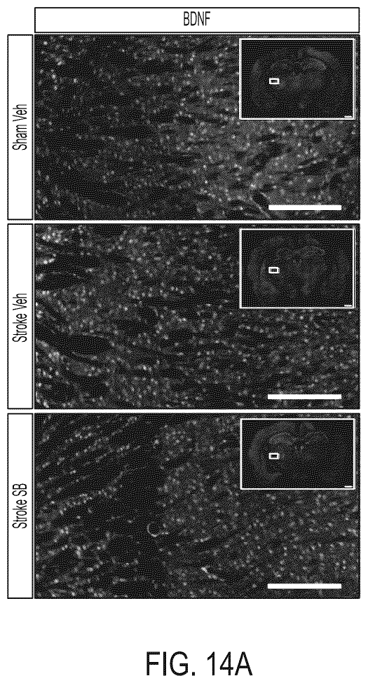

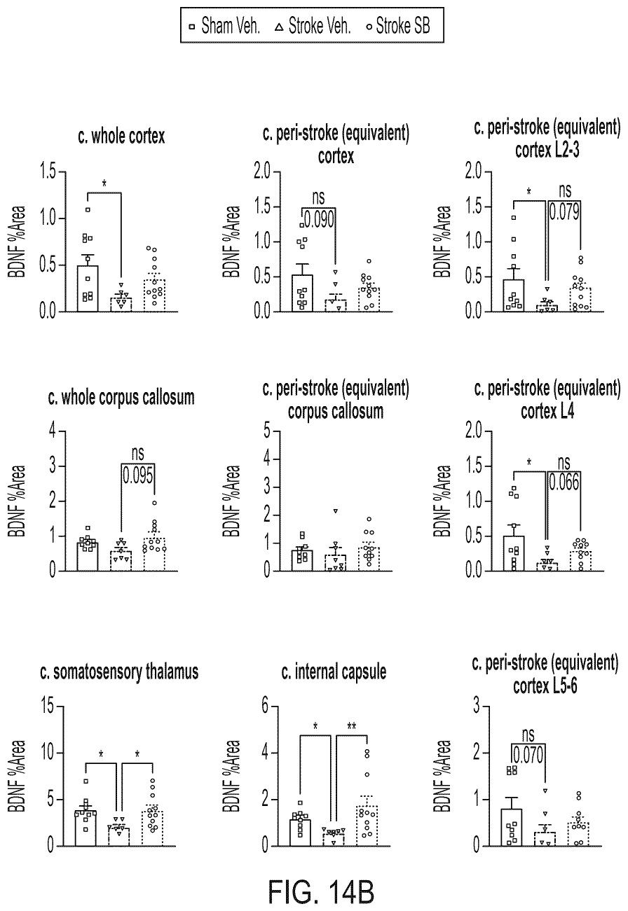

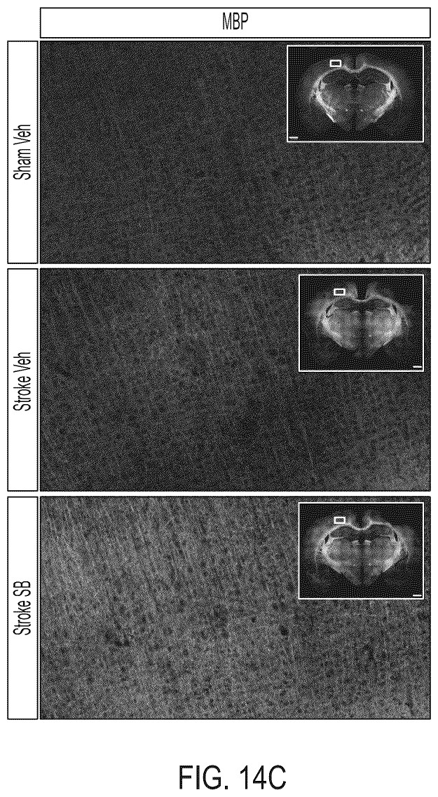

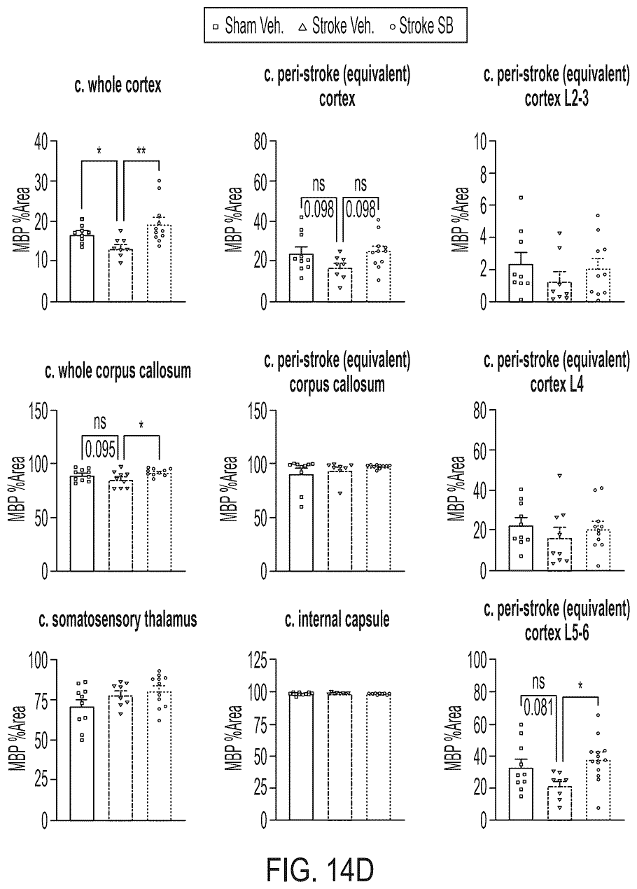

Interestingly, similar effects of stroke and vandefitemcel cell treatment/transplantation on BDNF expression/endogenous BDNF production were also observed in the contralesional hemisphere including the contralesional somatosensory thalamus (see FIGS. 14A and 14B), which shows that a local injection of the cells in the peri-stroke cortex also has remote effects.

In summary, the experimental results also showed that BDNF production was downregulated by stroke and upregulated by vandefitemcel in the peri-stroke cortex and in remote brain areas, such as the contralesional somatosensory thalamus.

FIG. 14A are images representing BDNF staining in the contralesional hemisphere in the location corresponding to the peri-stroke region in the ipsilesional hemisphere.

FIG. 14B illustrates the results of the staining illustrated in FIG. 14A, by the percentage of each contralesional ROI's area stained for BDNF in the sham vehicle (Sham Veh, squares), stroke vehicle (Stroke Veh, triangles) and stroke vandefitemcel (Stroke SB, circles) groups. Stroke significantly decreased the BDNF percentage area in the contralesional cortex (specifically also in the location corresponding to peri-stroke cortical layers 2-3 and 4), as well as the somatosensory thalamus and the internal capsule (with similar trends in the location corresponding to the whole cortical peri-stroke region, p=0.090, and therein also layers 5-6, p=0.071). Compared to the stroke vehicle group, after vandefitemcel treatment, there was significantly more BDNF in the contralesional somatosensory thalamus and the internal capsule (with similar trends in the whole corpus callosum, p=0.095, peri-stroke cortical layers 2-3, p=0.079, and layer 4, p=0.066).

BDNF also plays a role in synaptic plasticity, myelin repair, and other processes important for post-stroke recovery (reviewed in: Berretta et al., 2014; Liu et al., 2020a). A recent study in a rat MCAO model for ischemic stroke found that vandefitemcel cells administered at an acute time point post-stroke increased BDNF mRNA levels in the ischemic border zone, and even more so when combined with exercise (Yabuno et al., 2023). What was surprising from the results was that increased BDNF production was discovered not only in the peri-stroke area but also in more remote brain regions and at a chronic time point post-stroke. These findings are consistent with the hypothesis that vandefitemcel cell transplants stimulate synaptic plasticity via upregulation of BDNF production.

### Vandefitemcel Transplantation Induces Cellular Plasticity in Brain Tissue by Increasing Neuronal Progenitor Cells

One source of plasticity in the brain, which can potentially influence neuronal signaling, is neural progenitor cells. Doublecortin⁺ (DCX⁺) neuronal progenitor cells are generated from adult neural stem cells in the classic neurogenic niches in the subventricular zone and the hippocampal dentate gyrus, where some of them differentiate into mature neurons. Additionally, immature DCX⁺ cells can migrate. Their presence increases at sites of central nervous system (CNS) injury, and there is some evidence that this cell type is involved in neuro-recovery processes, even in the absence of neurogenesis, via release of neuroprotective and pro-regenerative factors (Jin et al., 2010; Ceanga et al., 2021; Campero-Romero et al., 2023).

FIG. 11C shows rat brain sections stained for DCX and FIG. 11D illustrates the results of the staining in FIG. 11C. FIG. 11D quantifies the percentage area of DCX staining in each ROI showing a significant stroke-induced increase in the ipsilesional hemisphere.

In comparison to vehicle injection, vandefitemcel cell treatment/transplantation significantly increased DCX staining in the ipsilesional cortex and corpus callosum as a whole and also in the peri-stroke region. In the neurogenic niche in the hippocampal dentate gyrus, neither stroke nor vandefitemcel cell treatment/transplantation induced significant DCX expression changes (see FIG. 11D).

In summary, vandefitemcel treatment increased the presence of DCX signal in the ipsilesional cortex and corpus callosum (also in the peri-stroke region). No significant DCX expression changes were found in the neurogenic niche in the hippocampal dentate gyrus.

### Vandefitemcel Transplantation Induces Cellular Plasticity in Brain Tissue by Increasing Oligodendrocyte Precursor Cells

Oligodendrocyte precursor cells (OPCs) are an abundant immature cell type in the adult CNS that can proliferate and differentiate to mature, post-mitotic, myelinating oligodendrocytes and thus contribute to myelin repair. OPCs also influence neuronal network activity by other mechanisms, such as the pruning of synapses, both glutamatergic and GABAergic synaptic signaling, and extracellular potassium homeostasis (reviewed in: Fang and Bai, 2023). To examine the oligodendrocyte lineage, rat brain section stains were conducted for oligodendrocyte transcription factor 2 (Olig2), a marker of both OPCs and mature oligodendrocytes, however, only OPCs are able to proliferate. Proliferating cell nuclear antigen (PCNA) was used to identify proliferating, PCNA+ and Olig2⁺ OPCs.

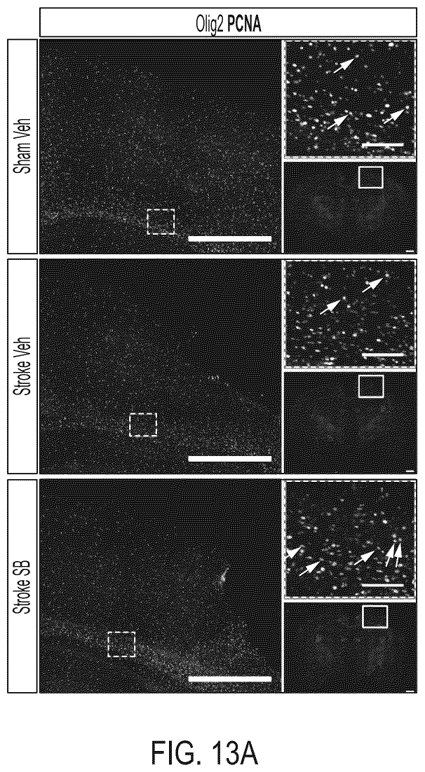

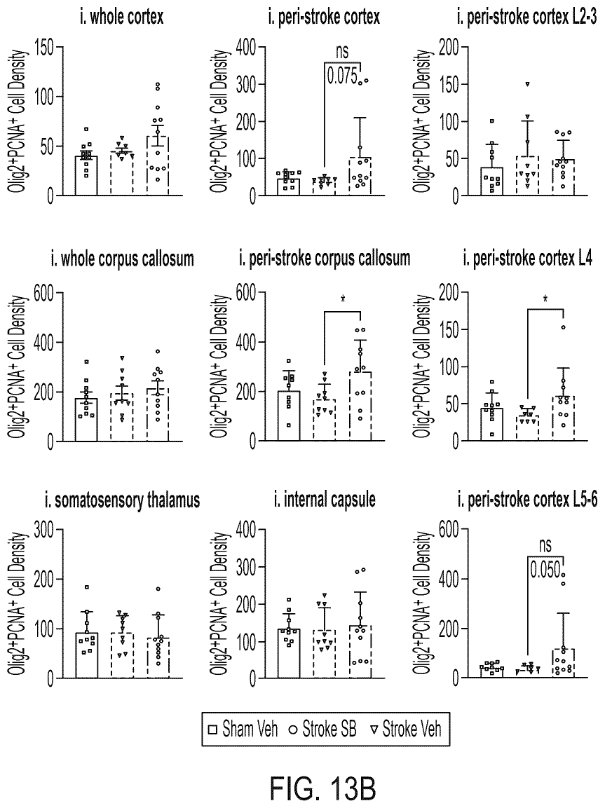

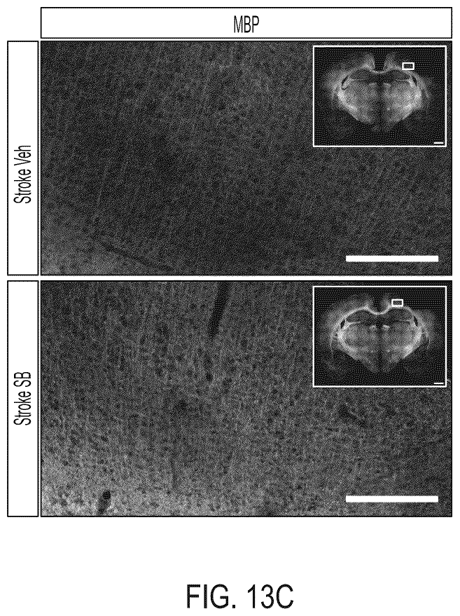

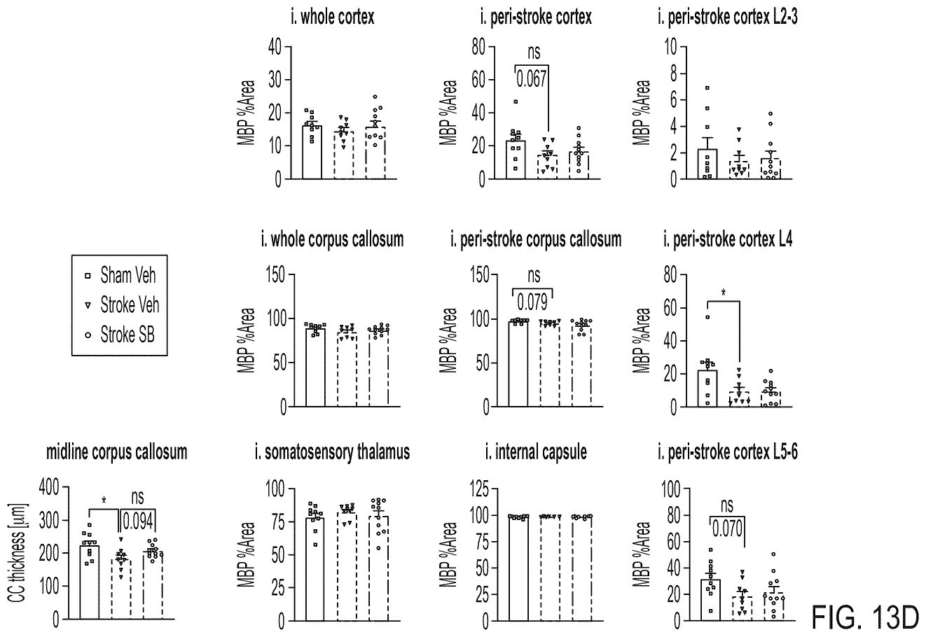

FIG. 13A are images showing Olig2 and PCNA staining in the peri-stroke region.

FIG. 13B illustrates the results of the staining illustrated in FIG. 13A, via an analysis of the density of proliferating oligodendrocyte precursor cells (OPCs) in each ROI in the sham vehicle (Sham Veh, squares), stroke vehicle (Stroke Veh, triangles) and stroke vandefitemcel (Stroke SB, circles) groups. Vandefitemcel treatment significantly increased the density of proliferating OPCs (Olig2⁺PCNA⁺; arrowheads) in the peri-stroke region in the corpus callosum and in cortical layer 4 (with a similar trend of p=0.050 in layers 5-6).

Neither stroke nor vandefitemcel cell treatment/transplantation had an effect on the total area of Olig2 staining in the brain regions analyzed. Stroke also did not change the density of proliferating OPCs (PCNA⁺, Olig2⁺). However, vandefitemcel cell treatment/transplantation significantly increased the density of proliferating OPCs (Olig2⁺PCNA⁺) in the peri-stroke corpus callosum and the peri-stroke cortex in layer 4 and a similar trend (p=0.050) was observed in layers 5-6 (see FIG. 13B). These results suggest that seven days post-treatment vandefitemcel cells caused a local increase in OPCs which might influence neural circuit activity in their progenitor stage and later may contribute to myelin repair once they differentiate to oligodendrocytes.

### Vandefitemcel Transplantation Induces Cellular Plasticity in Brain Tissue by Stimulating Myelin Remodeling

As previously discussed, cortical hyperexcitability reflects an increase of both the presynaptic and postsynaptic responses to white matter stimulation (see FIGS. 3A-3G and FIGS. 5A-5E). Presynaptic hyperexcitability suggests that stroke enhanced axonal excitability in the rats, which is consistent with reduced myelin binding protein (MBP) expression given that demyelination has been associated with axonal hyperexcitability (de Curtis et al., 2021). Such axonal hyperexcitability could explain, at least in part, the stronger postsynaptic responses in stroke rats compared with rats in the sham group.

Myelin basic protein (MBP) is a component of the myelin sheath. FIG. 13C are images representing staining of MBP in the ipsilesional hemisphere.

FIG. 13D illustrates the results of the staining shown in FIG. 13C, by the percentage of each ROI's area stained for MBP in the three experimental groups. A quantification of the MBP staining showed a significant stroke-induced decrease of MBP⁺ area in the peri-stroke cortex in layer 4. Similar trends were observed in the peri-stroke cortex in layers 5-6 (p=0.070) and the peri-stroke corpus callosum (p=0.079).

Vandefitemcel cell treatment/transplantation did not have any significant effects on MBP⁺ area in the regions analyzed in the ipsilesional hemisphere. Stroke also significantly reduced the midline thickness of the corpus callosum and vandefitemcel cell treatment/transplantation showed a slight trend toward reversing this effect (p=0.094) (see FIG. 13D).

Interestingly, stroke also induced a significant decrease in MBP staining in the contralesional cortex that was reversed by vandefitemcel cell treatment/transplantation. Similar trends were observed in other contralesional regions analyzed.

FIG. 14C are images representing MBP staining in the contralesional hemisphere in the location corresponding to the peri-stroke region in the ipsilesional hemisphere.

FIG. 14D illustrates the results of the staining illustrated in FIG. 14C, by the percentage of each contralesional ROI's area stained for MBP in the three experimental groups. Stroke significantly decreased the percentage area of MBP staining in the contralesional cortex (with similar trends in the contralesional corpus callosum, p=0.095, the region equivalent to the peri-stroke cortex, p=0.098, specifically also in layers 5-6, p=0.081). Compared to the stroke vehicle group, after vandefitemcel treatment, a significant increase of MBP percentage area was found in the contralesional cortex, specifically the region corresponding to the peri-stroke cortex layers 5-6, and the contralesional corpus callosum (with a similar trend, p=0.098, in the region corresponding to the whole peri-stroke cortex).

The effect of stroke and vandefitemcel cells on myelin remodeling in the contralesional hemisphere is interesting, as the contralesional hemisphere is involved in functional recovery after stroke, not only for motor abilities but also for somatosensory performance (Bestmann et al., 2010; Volz et al., 2017; Grefkes and Fink, 2020; Lv et al., 2022).

The experimental results disclosed herein are the first to show that vandefitemcel cell treatment/transplantation increased OPC proliferation and stimulated myelin remodeling (via an increase in MBP) in vivo. Since BDNF stimulates myelin plasticity and repair (reviewed in Fletcher et al., 2018), it is possible that the vandefitemcel-induced increase of endogenous BDNF production that was discussed in the earlier section facilitates the cells' effects on OPCs and myelin.

### Vandefitemcel Transplantation Induces Cellular Plasticity in Brain Tissue by Increasing Astrocytes and Microglia

Astrocytes modulate neuronal signaling by regulating synaptic transmission and synaptogenesis, recycling neurotransmitters, for example via GAT-1, and maintaining extracellular ion homeostasis (reviewed in: Sofroniew, 2020; Xiong et al., 2022; Bazargani and Attwell, 2016; Nagai et al., 2021). Another important function of astrocytes is to support recovery after CNS damage, for example by producing trophic factors such as BDNF and providing metabolic support for neurons (reviewed, for example, in: Linnerbauer and Rothhammer, 2020; Nagai et al., 2021).

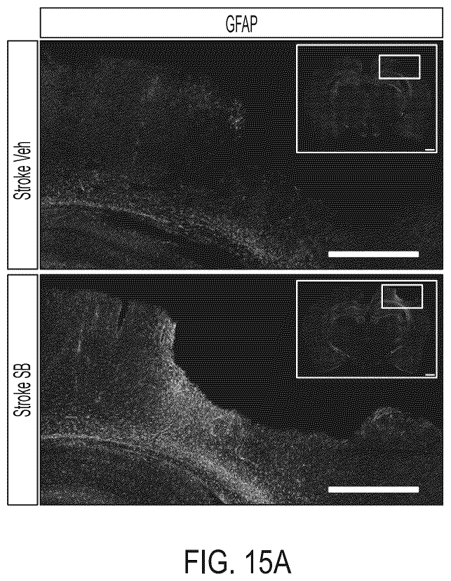

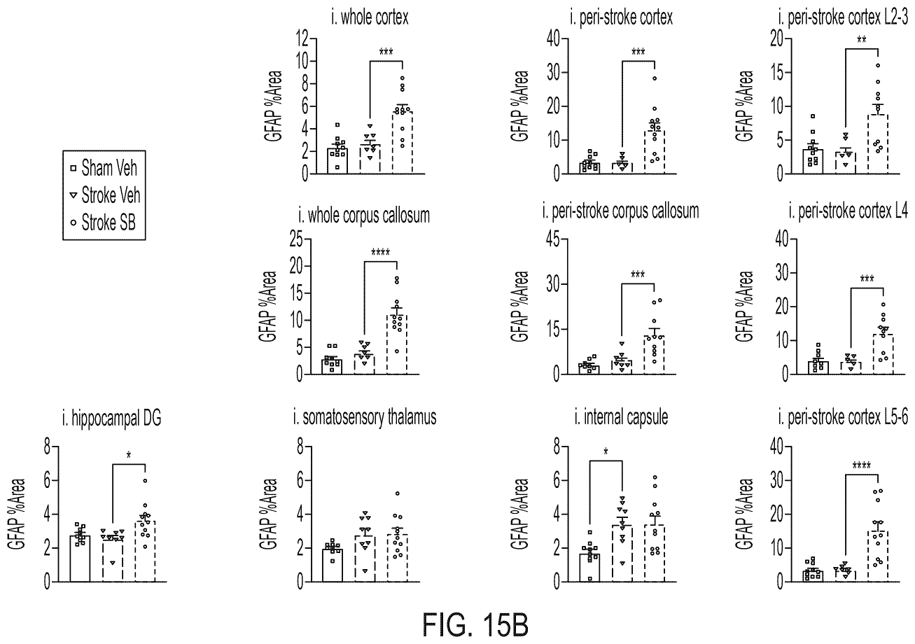

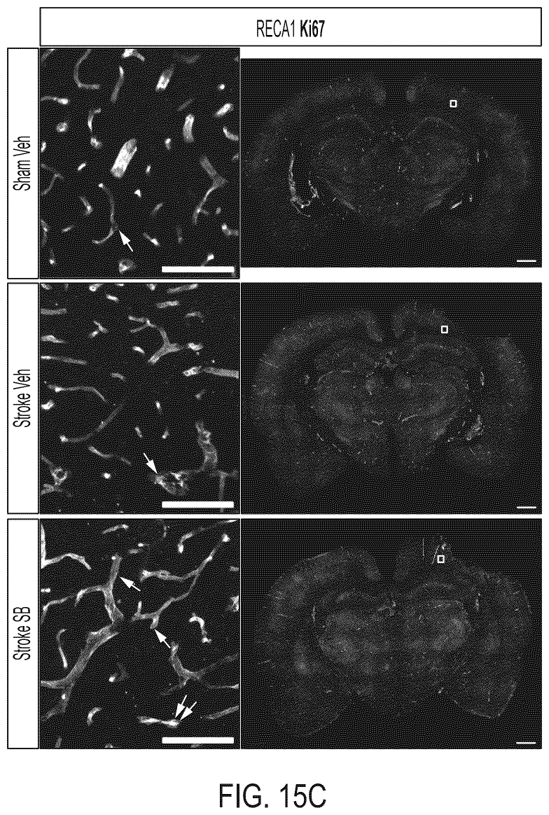

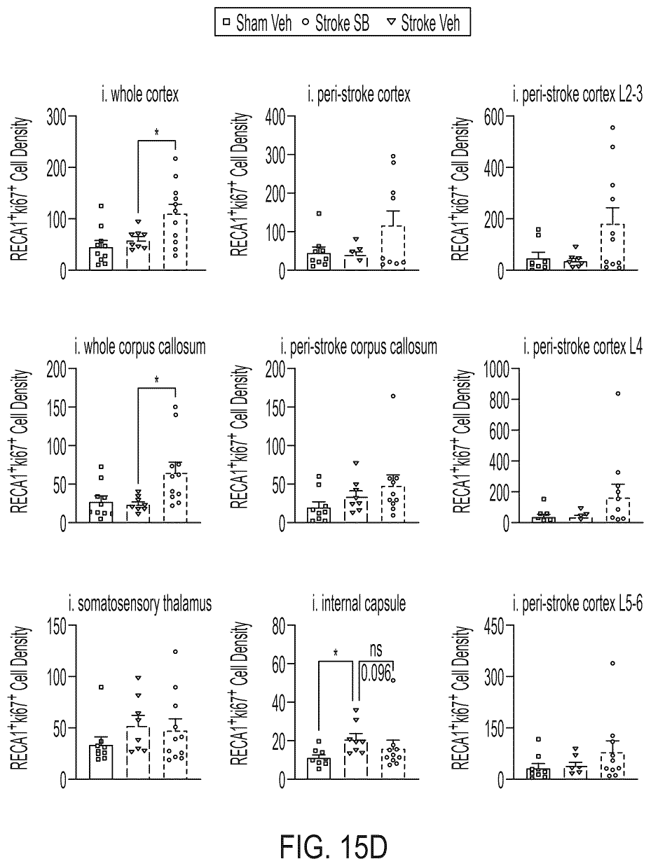

FIG. 15A are images representing glial fibrillary acidic protein (GFAP) staining in the peri-stroke region.

FIG. 15B illustrates the results of the staining illustrated in FIG. 15A, by the percentage of each ROI's area stained for GFAP in the sham vehicle (Sham Veh, squares), stroke vehicle (Stroke Veh, triangles), and stroke vandefitemcel (Stroke SB, circles) groups. Stroke significantly increased the GFAP⁺ percentage area only in the ipsilesional internal capsule. After vandefitemcel treatment, there was a significantly higher GFAP⁺ percentage area of astrocytes in the cortex and the corpus callosum (and in all peri-stroke subregions analyzed).

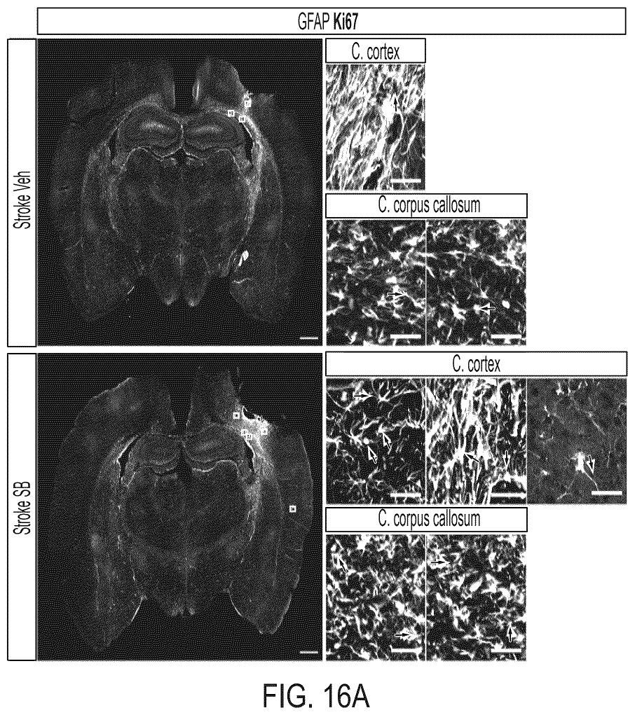

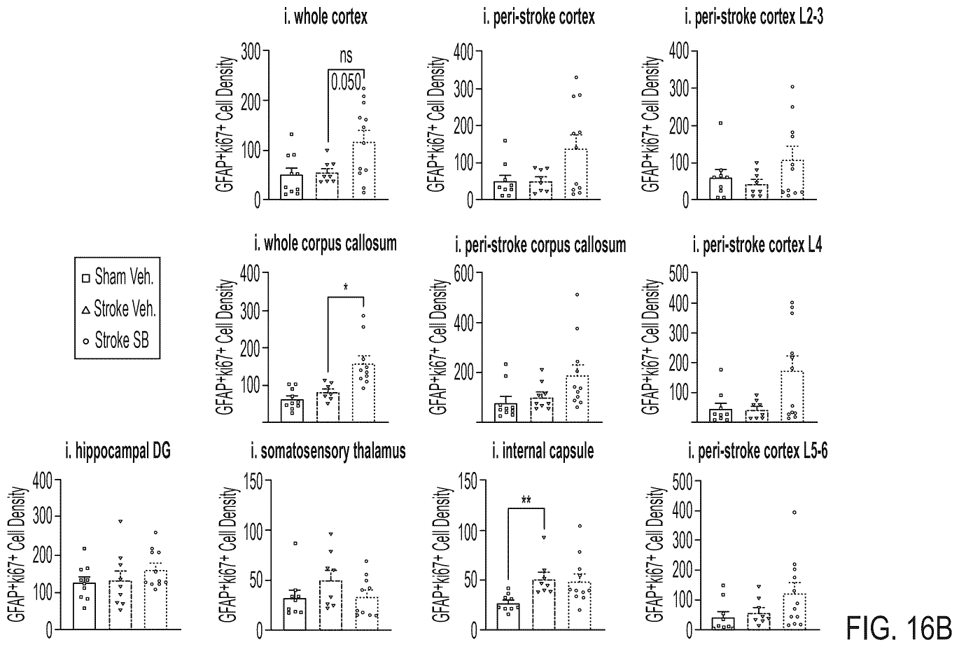

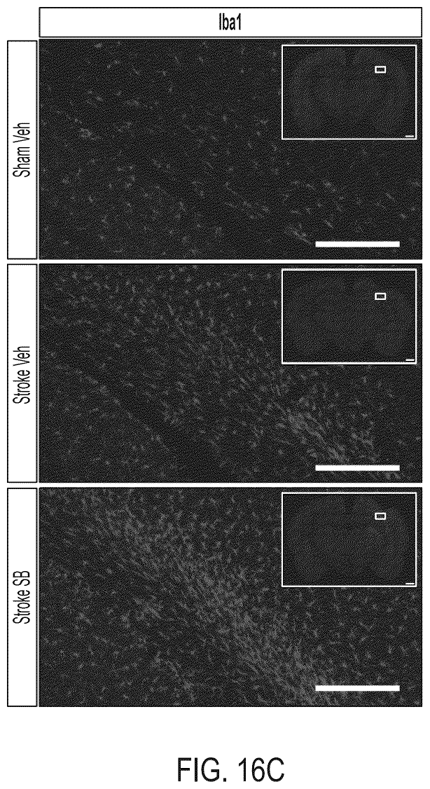

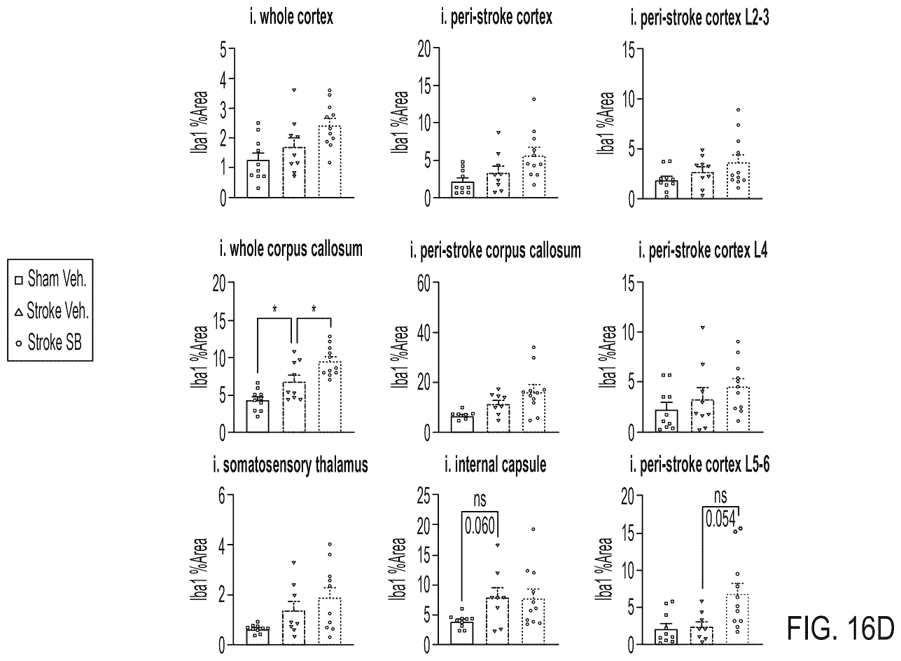

Interestingly, staining for the astrocyte marker GFAP (see FIGS. 15A and 15B) showed that stroke had no detectable effect on the expression of this marker (see FIGS. 15A and 15B) or the density of proliferating astrocytes (ki67⁺ and GFAP⁺; see FIGS. 16A and 16B) in any of the ROIs examined except in the ipsilesional internal capsule, where it significantly increased staining. Seven days after administration/transplantation into the peri-stroke cortex, vandefitemcel cell treatment/transplantation induced a clear increase in expression of the astrocyte marker GFAP in the ipsilesional cortex and corpus callosum, and in the peri-stroke region (see FIGS. 15A and 15B).

FIG. 16A are images representing staining for ki67⁺ and GFAP⁺ in the peri-stroke region (arrowheads within the staining plots show examples for double-positive cells).

FIG. 16B illustrates the results of the staining illustrated in FIG. 16A, by the density of proliferating GFAP⁺ astrocytes in each ROI in the sham vehicle (Sham Veh, squares), stroke vehicle (Stroke Veh, triangles) and stroke vandefitemcel (Stroke SB, circles) groups. In the stroke vehicle group, the density of ki67⁺ and GFAP⁺ cells was significantly higher than in the sham vehicle group in the ipsilesional internal capsule. In the stroke SB group, the density of proliferating GFAP⁺ astrocytes was significantly increased in the ipsilesional corpus callosum and, as a trend of p<0.050, also in the ipsilesional cortex compared to the stroke vehicle group.

A higher density of proliferating GFAP⁺ astrocytes indicates that this GFAP increase is not only due to morphological changes but also an increase in astrocyte numbers (see FIGS. 16A and 16B).

FIG. 16C are images representing ionized calcium-binding adapter molecule 1 (Iba1) staining in the peri-stroke region.

FIG. 16D illustrates the results of the staining illustrated in FIG. 16C, showing that compared to the sham vehicle group, the stroke vehicle group Iba1⁺ percentage area was significantly higher the ipsilesional corpus callosum, and for the ipsilesional internal capsule a similar trend (p=0.060) was found. In comparison to the stroke vehicle group, there was a further increase of Iba1⁺ percentage area in the stroke vandefitemcel group in the ipsilesional corpus callosum, and a trend (p=0.060) for an Iba1 increase in layers 5-6 of the peri-stroke cortex.

Stroke significantly increased Iba1⁺ microglia area in the ipsilesional corpus callosum, and a similar trend was observed in the ipsilesional internal capsule (p=0.060). Vandefitemcel cell treatment/transplantation further significantly increased Iba1⁺ microglia area in the ipsilesional corpus callosum and also caused a trend (p=0.054) for an Iba1⁺ increase in the peri-stroke cortex in layers 5-6 (see FIG. 16D).

The current discovery that vandefitemcel treatment leads to an increase in GFAP⁺ astrocytes as well as Iba1⁺ microglia/macrophages, seven days after intracortical administration, is consistent with the time sequence that has been described for intracerebral MSC treatment. For MSC grafts into the CNS, the sequence of immune cell infiltration and recruitment of astrocytes and microglia/macrophages in the days after cell transplantation has been established, and it has been hypothesized that this endogenous response of immune cells and glia contributes to the therapeutic regenerative effects (Le Blon et al., 2017).

FIG. 15C are images representing staining of RECA1 and ki67 in the ipsilesional hemisphere.

FIG. 15D illustrates the results of the staining illustrated in FIG. 15C, by an analysis of the density of proliferating OPCs in each ROI in the three groups in the experiment.

Stroke led to a significantly higher density of proliferating RECA1⁺ endothelial cells in the ipsilesional internal capsule. Vandefitemcel treatment significantly increased the density of proliferating endothelial cells in the whole cortex and corpus callosum and there was a slight trend (p=0.096) for a reduction of the density of RECA1+ and ki67⁺ cells in the internal capsule.

Restoration of blood supply through angiogenesis is important for stroke recovery (Fang et al., 2023). Staining of RECA1, a marker for endothelial cells (see FIG. 15C) did not show any significant changes in the total area that was RECA1⁺, but stroke significantly increased the density of proliferating (ki67⁺) RECA1⁺ endothelial cells in the ipsilesional internal capsule. Vandefitemcel cell treatment/transplantation significantly increased the density of proliferating RECA⁺ki67⁺ endothelial cells in the cortex and corpus callosum as a whole and showed a slight trend (p=0.096) for a reduction of the density of RECA1⁺ki67⁺ cells in the ipsilesional internal capsule.

A proangiogenic effect of vandefitemcel cells has been found in vitro (Dao et al., 2013). Treatment with vandefitemcel cells increased arteriogenesis and improved tissue perfusion (the latter starting as early as 2 weeks post-treatment) in a rat hindlimb ischemia model (Maeda et al., 2021), and two weeks after vandefitemcel cell administration into a rat MCAO ischemic stroke model in the acute phase post-stroke, more laminin-labeled blood vessels were found in the ischemic border zone (Yabuno et al., 2023). While there was no overall change in the presence of RECA1⁺ endothelial cells, a significant increase in the density of proliferating endothelial cells in the ipsilesional cortex and corpus callosum was discovered after vandefitemcel cell treatment/transplantation (see FIG. 15D), which might indicate that 7 days post-treatment, angiogenesis is starting and might lead to improved vessel coverage at later time points.

### Vandefitemcel Transplantation Reverses Stroke-Induced Changes in Whole-Blood Gene Expression

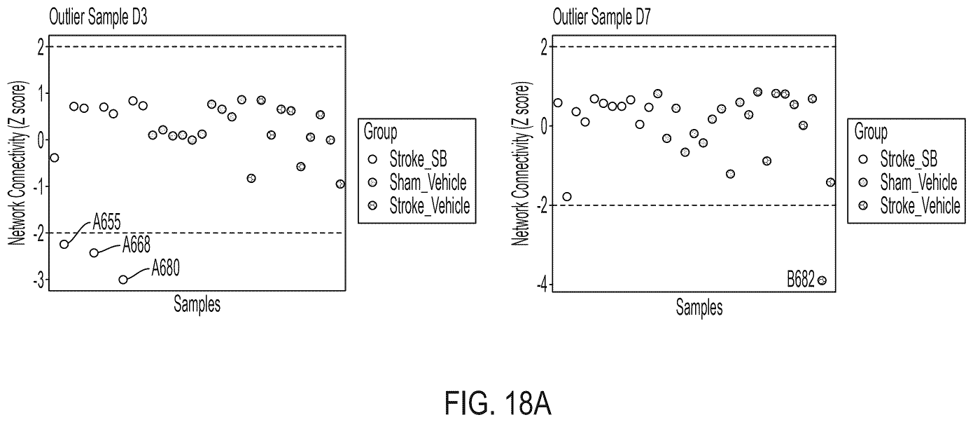

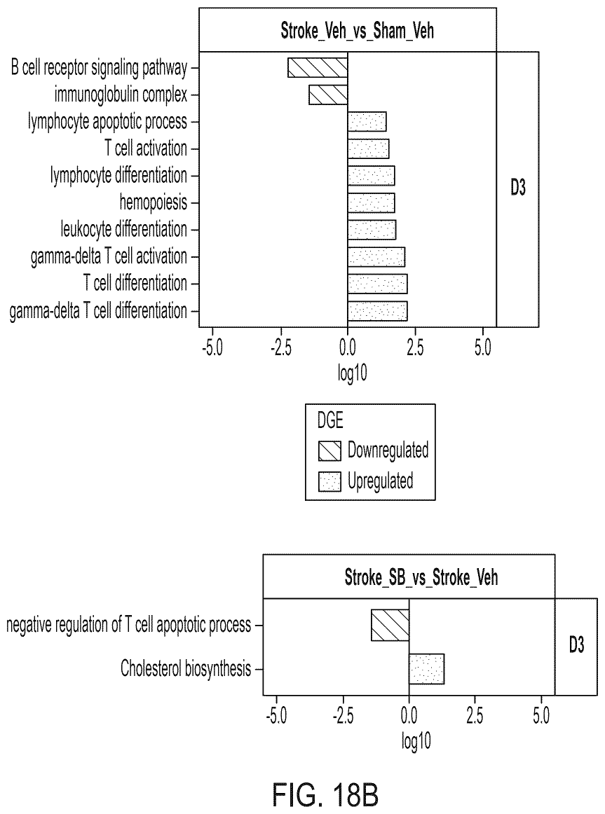

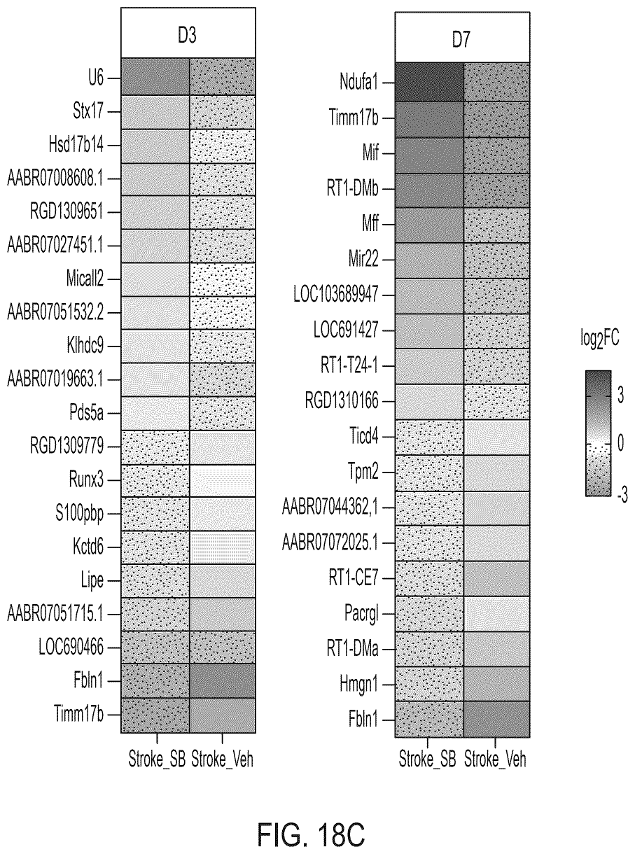

Next, experiments were undertaken to determine the extent of stroke-induced changes in the peripheral blood, in particular in the peripheral immune system, and to what degree these changes are reversed by the cell transplants. To this end, whole-blood RNA was collected 3 days and 7 days after the cell transplant or vehicle treatment, and it was analyzed via bulk RNAseq (for visualization of outlier analysis see FIG. 18A).

FIG. 18A (left panel) is a plot with sample outlier removal performed at Day 3 and FIG. 18A (right panel) is a plot with sample outlier removal performed at Day 7 based on Z-scores of standardized network connectivity.

Stroke had a considerable impact on gene expression in the peripheral blood on both analysis days. The number of genes significantly affected by vandefitemcel cell treatment/transplantation, on both Day 3 and Day 7 post-treatment, was smaller than the number of genes significantly affected by stroke.

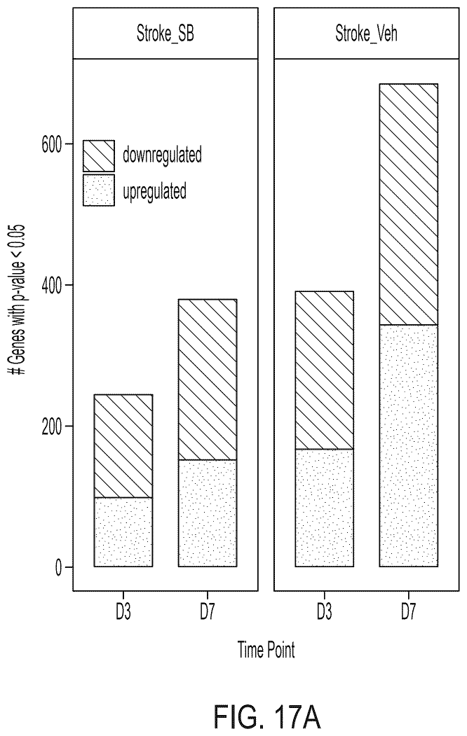

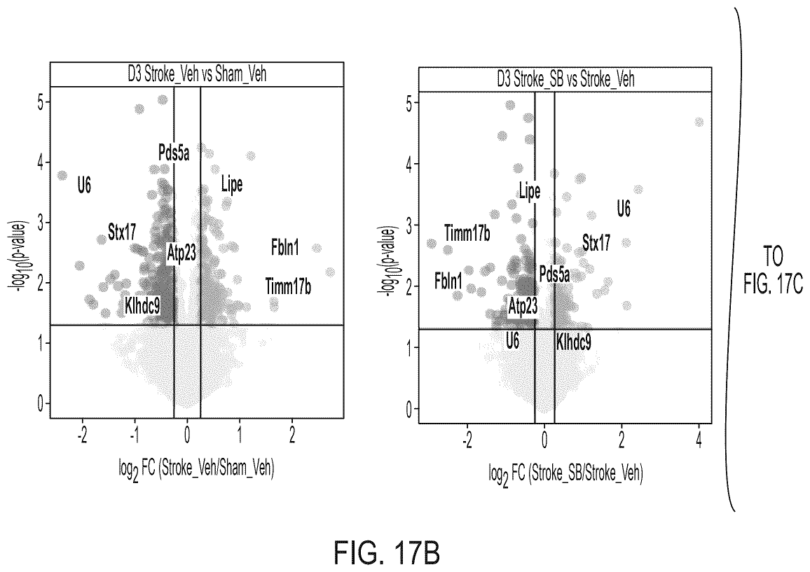

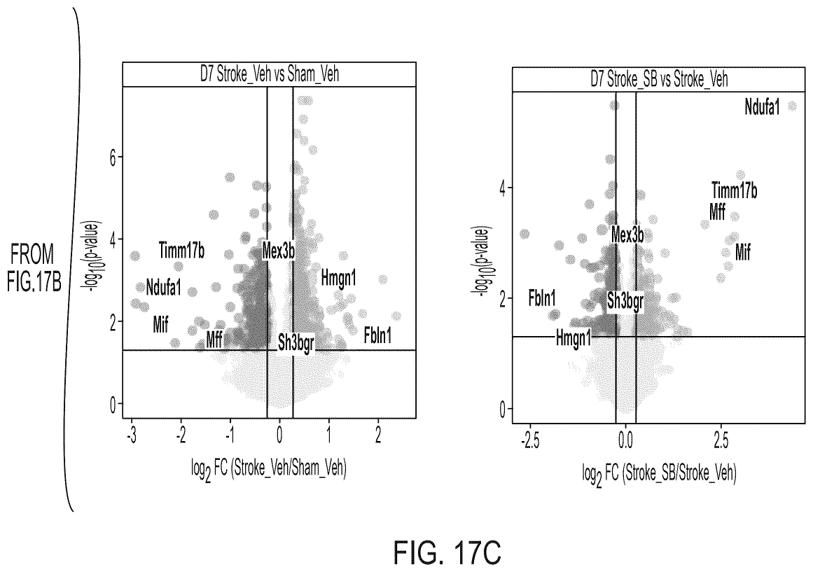

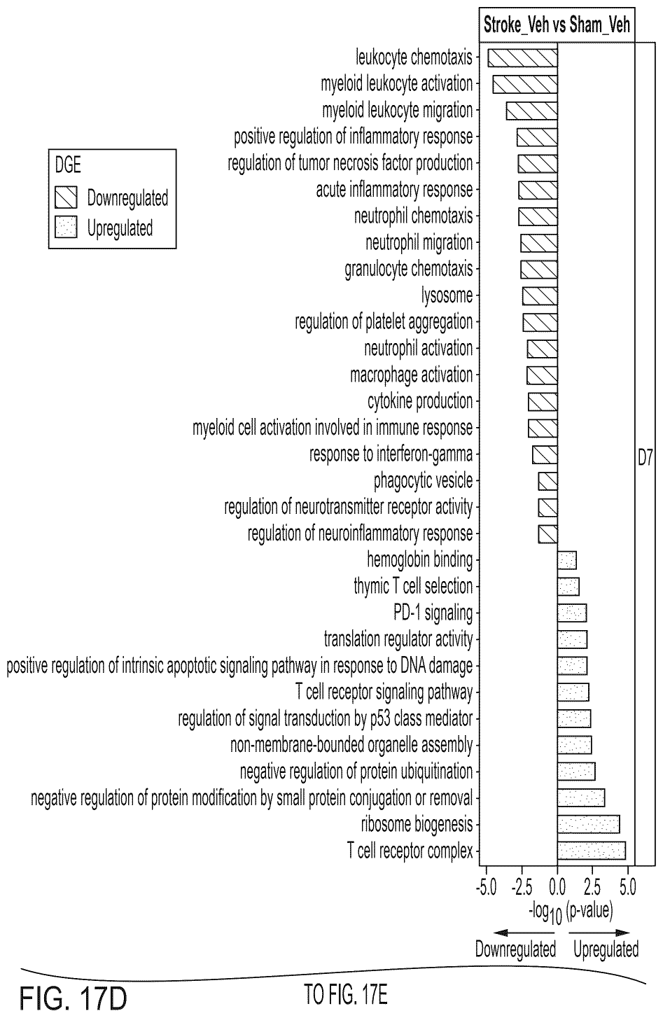

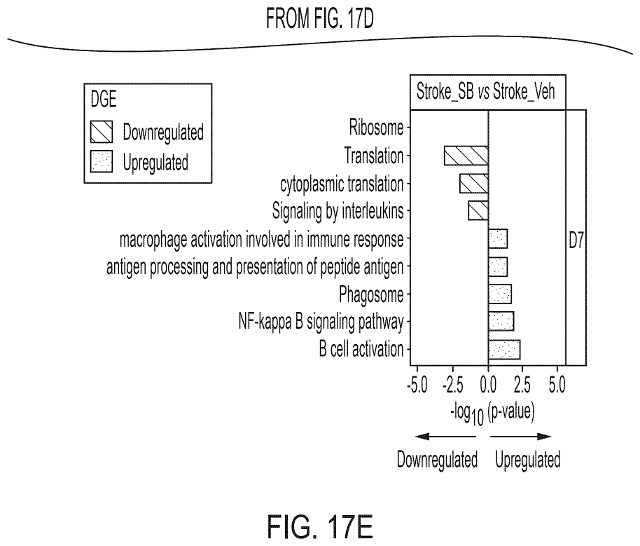

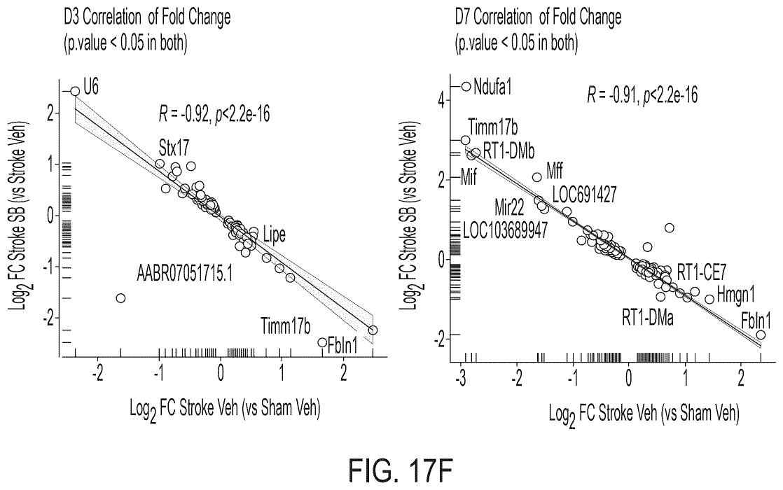

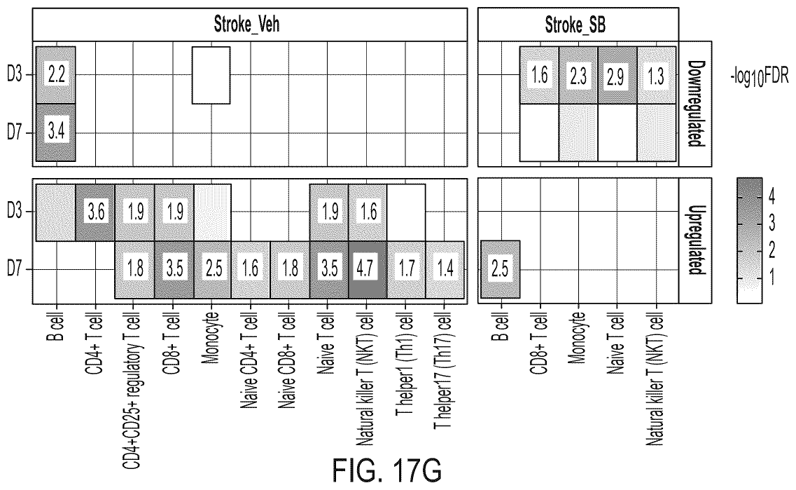

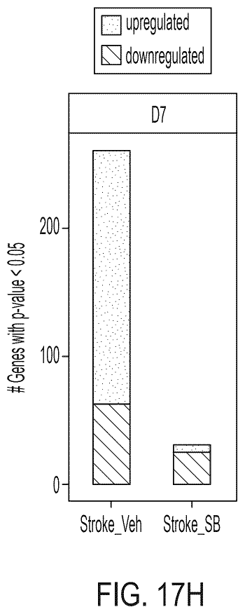

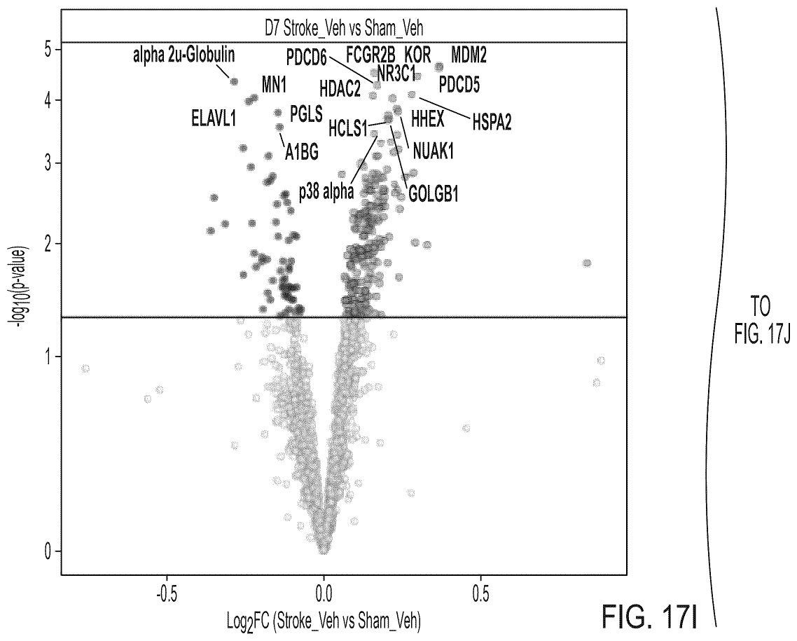

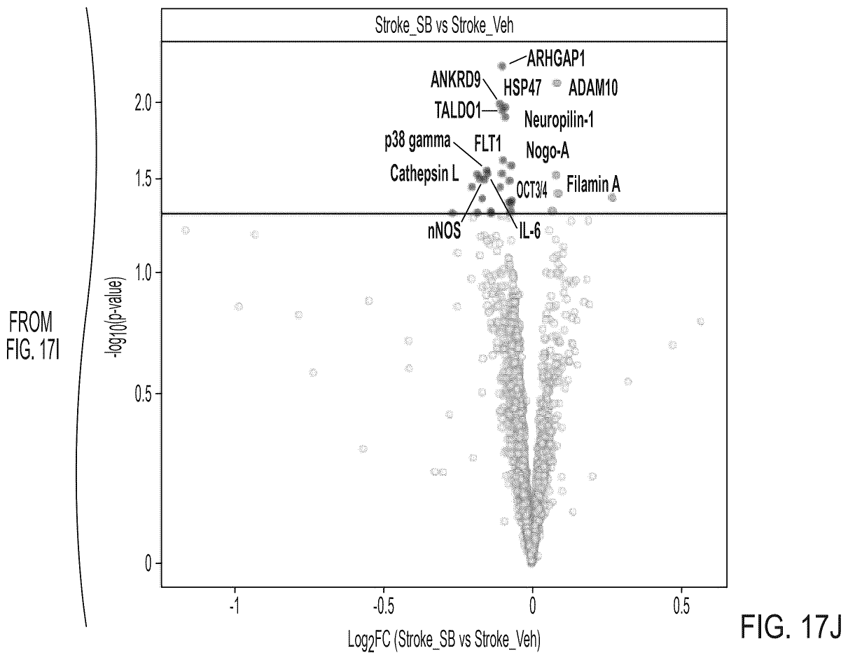

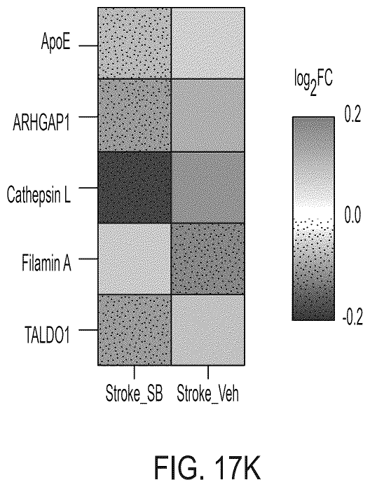

FIG. 17A (left panel) shows the number of whole-blood genes significantly downregulated and upregulated in the stroke vandefitemcel group (Stroke_SB) compared to the stroke vehicle (Stroke_Veh) group on Day 3 (D3) and Day 7 (D7) after treatment. FIG. 17A (right panel) shows the same comparison between the stroke vehicle group and the sham vehicle group. Data represents 7-10 rats per group on Day 3 and 9-11 rats per group on Day 7.

FIGS. 17B and 17C are volcano plots of differentially expressed genes for the stroke vehicle group compared to the sham vehicle group (Sham_Veh) and the stroke vandefitemcel group compared to the stroke vehicle group for Day 3 and Day 7. Downregulated genes are represented in darker shading and unregulated genes are represented in lighter shading. The x-axes show the log₂ fold-change (FC) of the differentially expressed genes and the y-axes the −log₁₀ of the p-value.

FIG. 18B illustrates a pathway analysis of differentially expressed whole-blood genes on blood collection Day 3 post-treatment, with stroke vehicle vs. sham vehicle graphs (top) and stroke vandefitemcel vs. stroke vehicle graphs (bottom). The contribution of upregulated or downregulated proteins to specific GO, KEGG, or Reactome terms are shown, respectively. The pathway enrichment analysis showed that more pathways were significantly changed by stroke than by vandefitemcel treatment on post-treatment Day 3, on which overall fewer pathways were significantly enriched (see FIG. 18B), and Day 7 (see FIGS. 17D and 17E).

FIGS. 17D and 17E display the topmost relevant Gene Ontology (GO), Kyoto Encyclopedia of Genes and Genomes (KEGG), and Reactome pathways for up-regulated and down-regulated genes in the differential gene expression (DGE) analysis at Day 7, respectively, in two comparisons: stroke vehicle vs. sham vehicle (FIG. 17D) and stroke vandefitemcel vs. stroke vehicle (FIG. 17E). These enrichments highlight the biological processes most significantly affected by stroke or treatment with vandefitemcel cells compared to either sham or vehicle controls.

The pathway enrichment analysis suggested that some stroke-induced changes in the blood (for example, a downregulation of pathways associated with phagocytic vesicles and lysosome) are counteracted by administration of vandefitemcel cells into the peri-stroke cortex (which, for example, upregulated a pathway related to phagocytosis). For this reason, a correlation analysis was conducted between all significantly changed genes on Day 3 and Day 7 after treatment with either vehicle or vandefitemcel cells.

FIG. 17F illustrates a correlation of fold change between the stroke effect (comparison stroke vehicle vs. sham vehicle group) on the x-axis and the vandefitemcel effect (comparison stroke vandefitemcel vs. stroke vehicle) on the y-axis in genes that are nominally significant (p<0.05) for both effects after treatment one-month post-stroke. The left panel of FIG. 17F shows results for Day 3 and the right panel of FIG. 17F shows results for Day 7.

This analysis showed a highly significant negative correlation between genes significantly changed by stroke (in the comparison between the stroke vehicle group and the sham vehicle group) and those significantly changed by vandefitemcel cell treatment/transplantation (in the comparison between the stroke vandefitemcel group and the stroke vehicle group) on Day 3 as well as on Day 7 post-treatment (see FIG. 17F).

Based on these results, a list of “rescued” genes was generated, which were defined as having significant stroke-induced expression changes that were significantly counteracted (or “rescued”) by the vandefitemcel cell treatment/transplantation on one of the blood collection days.

FIG. 18C illustrates the top 20 genes that were induced by stroke and rescued by vandefitemcel treatment in the peri-stroke cortex, showing Day 3 (left) and Day 7 (right) examples of stroke vandefitemcel vs. stroke vehicle groups. This list of “rescued” genes contained 29 upregulated stroke genes that were downregulated by vandefitemcel cells on Day 3, and 82 such genes on Day 7, and 26 and 58 genes that were upregulated by stroke and downregulated by vandefitemcel cells on Day 3 and Day 7 post-treatment, respectively.

The top 20 “rescued” genes on Day 3 and Day 7 (see FIG. 18C) include U6, Stx 17, Hsd17b14, AABR07008608.1, RGD1309651, AABR07027451.1, Micall2, AABRO7051532.2, Klhdc9, AABR07019663.1, Pds5a, RGD1309779, Runx3, S100pbp, Kctd6, Lipe, AABRO7051715.1, LOC690466, Fbin1, Timm17b on Day 3 and Ndufa1, Timm17b, Mif, RT1-DMb, Mff, Mir22, LOC103689947, LOC691427, RT1-T-24-1, RGD1310166, Ticd4, Tpm2, AABR07044362.1, AABR07072025.1, RT1-CE7, Pacrgl, RT1-DMa, Hmgn1, and Fbin1.

Among the stroke-induced changes that were “rescued” by vandefitemcel cell treatment/transplantation was, for example, CD8b, which is expressed by cytotoxic T cells and was downregulated in the stroke vandefitemcel group on both Day 3 and Day 7. This is in line with an in vitro study that demonstrated immunomodulatory properties of vandefitemcel cells, including the inhibition of activation and proliferation of T cells (Dao et al., 2011). Other examples of genes that were downregulated by vandefitemcel cells on Day 3 post-treatment include Scarf1 and Dusp1 that have been described as stroke “hub genes” (Wu et al., 2022; Yang et al., 2022; Zhou et al., 2023). In a proteome-wide and transcriptome-wide association study, Scarf1 was identified as one of five genes in human blood with causal relationships to stroke and its subtypes (Wu et al., 2022). Dusp1 was found among five identified hub genes involved in the pathogenesis of ischemic stroke in the peripheral blood of patients (Yang et al., 2022), and as one of nine hub genes in patients' whole-blood mRNA involved in oxidative stress after ischemic stroke (Zhou et al., 2023). Another gene that was “rescued” (downregulated) by vandefitemcel cells was Csnk2a1, one of two genes encoding the catalytic subunit of protein kinase CK2, on Day 3 post-treatment. Targets of CK2 play important roles in several neurodegenerative diseases, such as Alzheimer's disease and Parkinson's disease, in which CK2 activity may contribute to disease pathology (e.g., by promoting Tau hyperphosphorylation or alpha synuclein aggregation) but also have protective effects (e.g., by reducing Abeta production) (reviewed in: Borgo et al., 2021). It is interesting that vandefitemcel cell treatment/transplantation not only modulates stroke-induced changes in expression of genes with known associations with stroke pathophysiology, but also with neurodegenerative diseases.

A co-expression analysis of the DGE results showed three modules that were significantly changed after vandefitemcel cell treatment/transplantation in comparison to vehicle treatment.

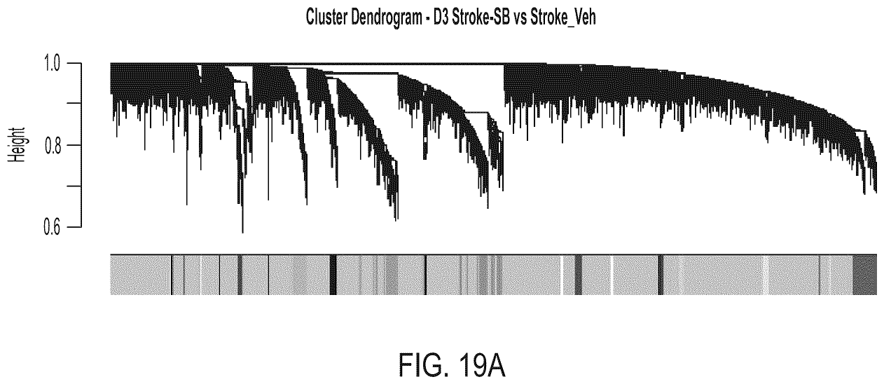

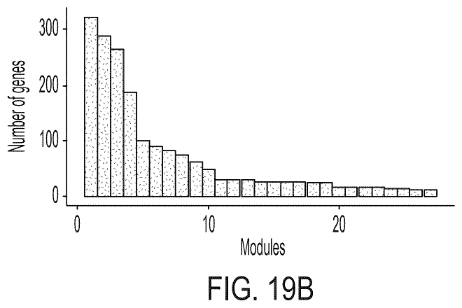

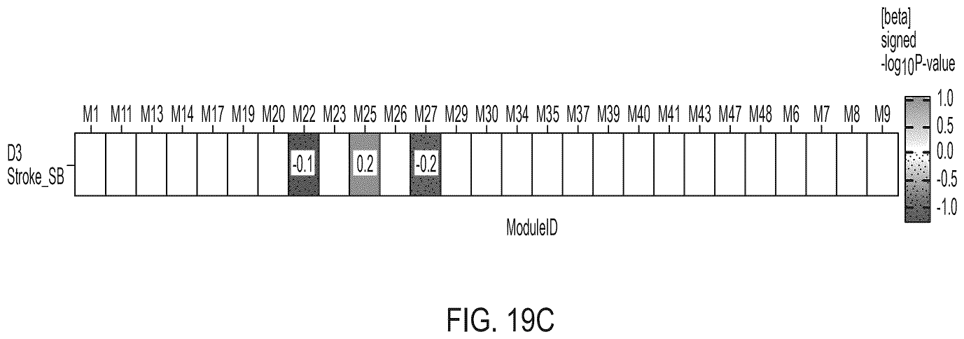

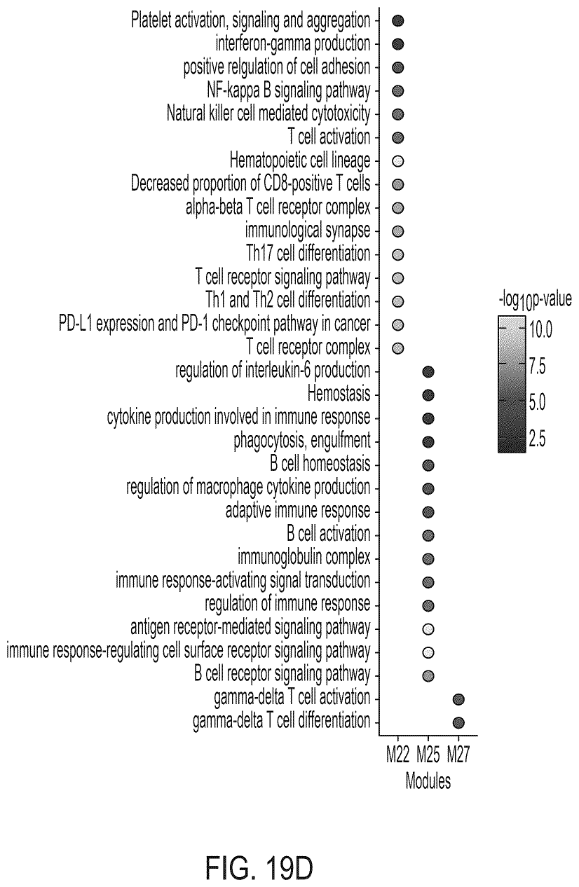

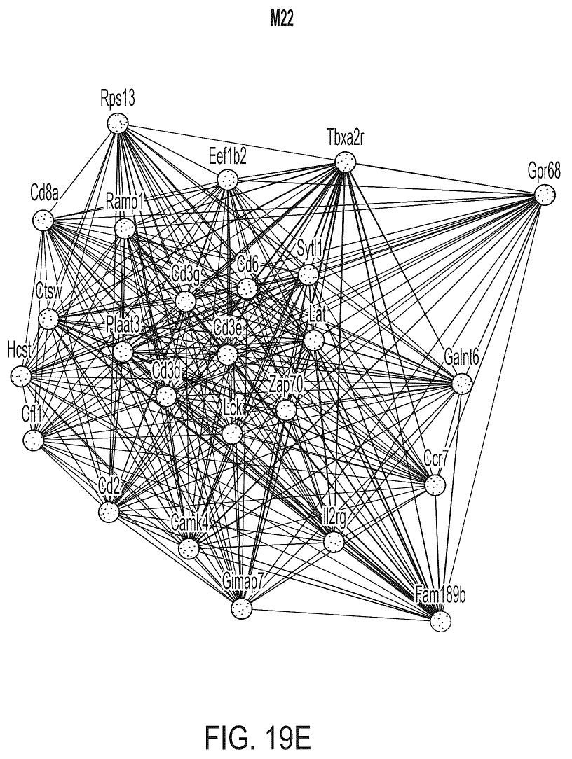

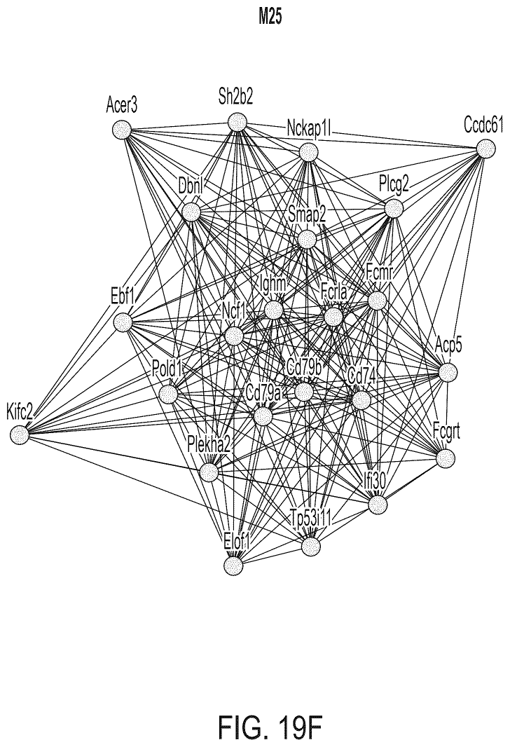

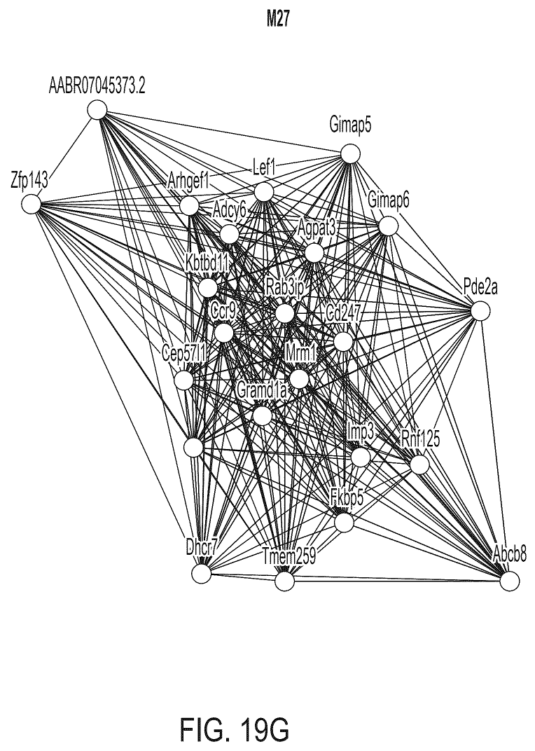

FIG. 19A illustrates a clustering dendrogram of genes with gene dissimilarity based on topological overlap on Day 3 post-treatment. FIG. 19B illustrates the number of genes per co-expression module detected on Day 3 post-treatment. FIG. 19C illustrates significant modules associated with the comparison stroke vandefitemcel group vs. the stroke vehicle group on blood collection Day 3 post-treatment. Modules 22 and 27 are downregulated while module 25 is upregulated for the stroke vandefitemcel group.

FIG. 19D illustrates the topmost relevant GO, KEGG, WikiPathways (WP), and Reactome upregulated and downregulated pathways for significant co-expression modules (modules 22, 25, and 27). Selected pathways have a minimum of 3 associated genes (intersect 22>=3).

FIG. 19E illustrates a network of the most connected genes (“eigengenes”) in module 22. FIG. 19F illustrates a network of the most connected genes (“eigengenes”) in module 25. FIG. 19G illustrates a network of the most connected genes (“eigengenes”) in module 27.

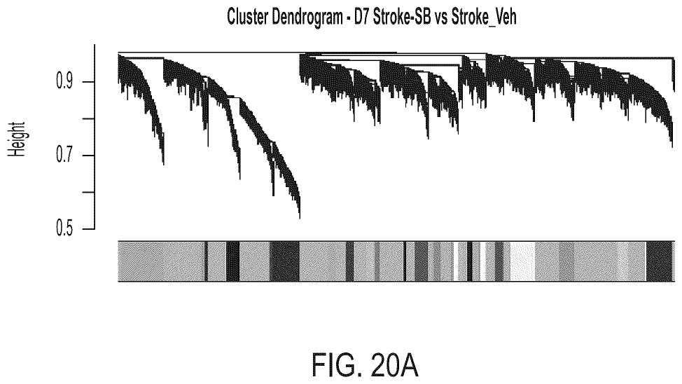

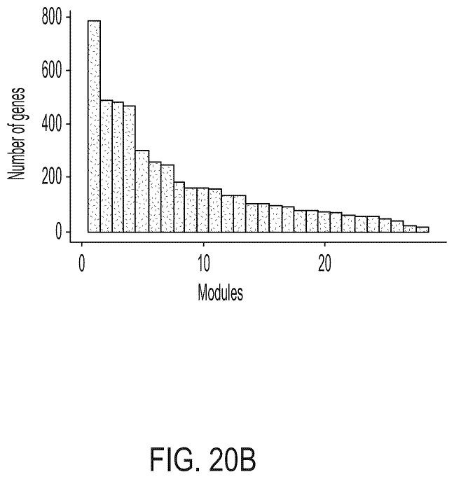

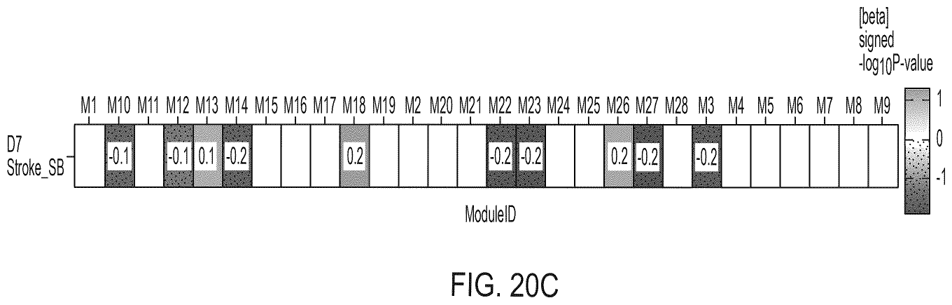

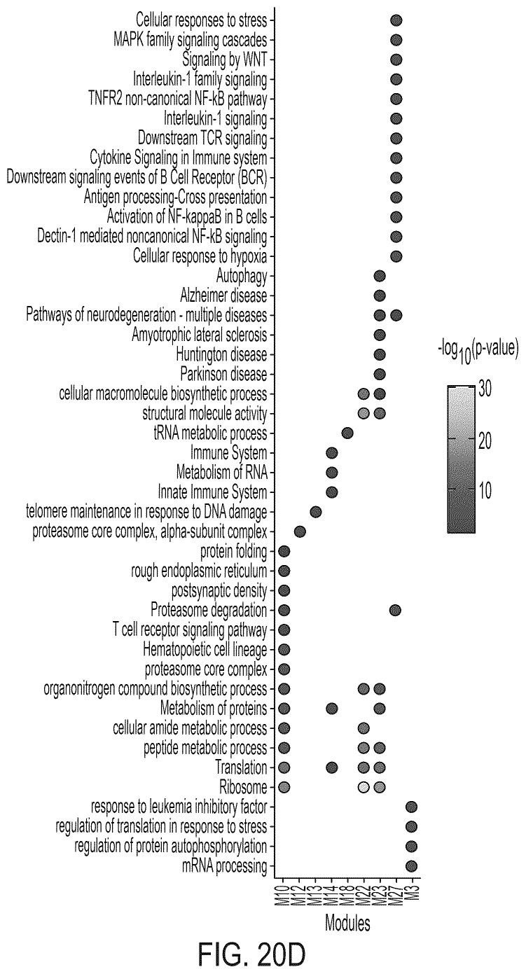

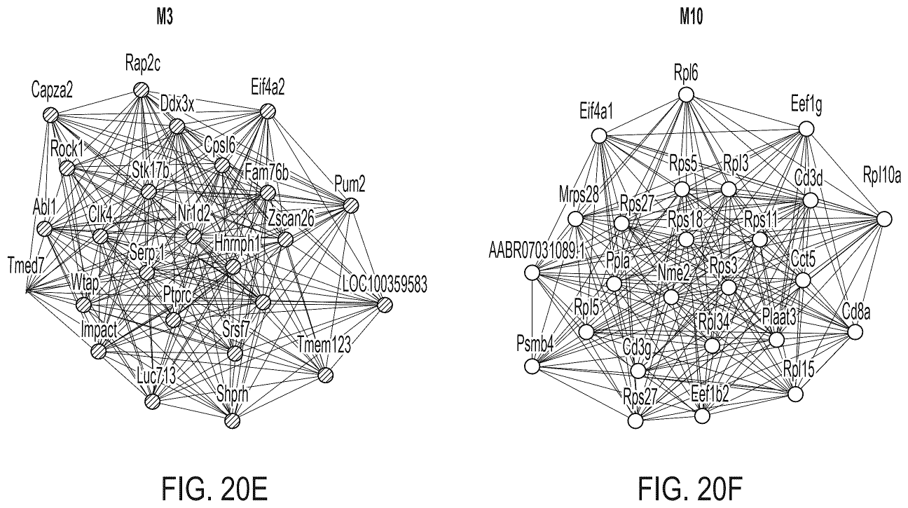

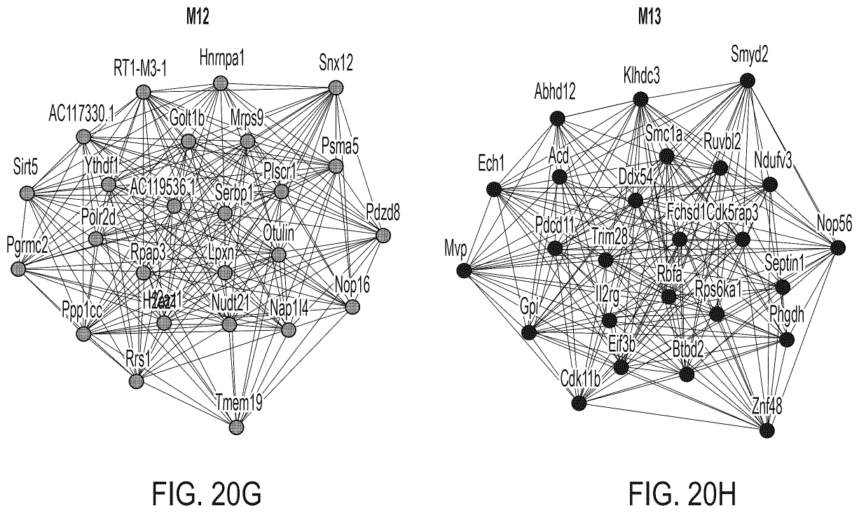

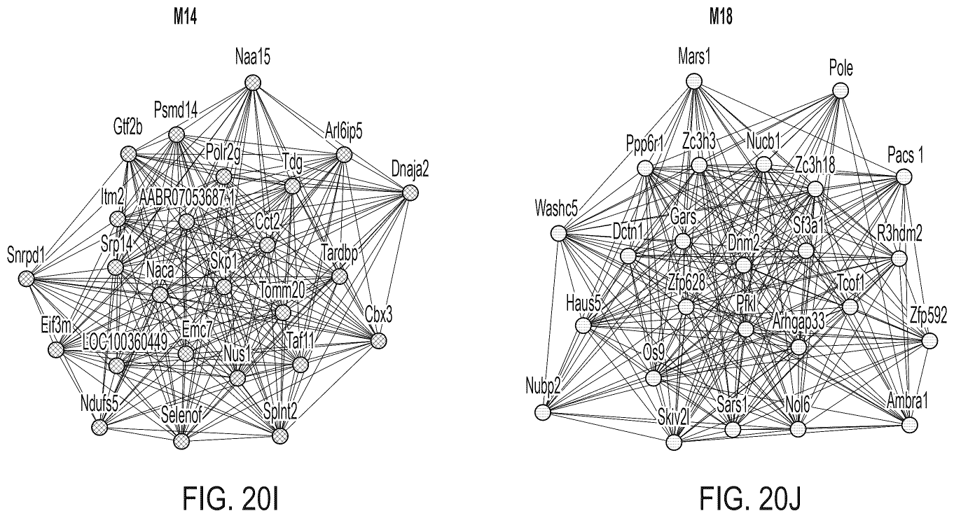

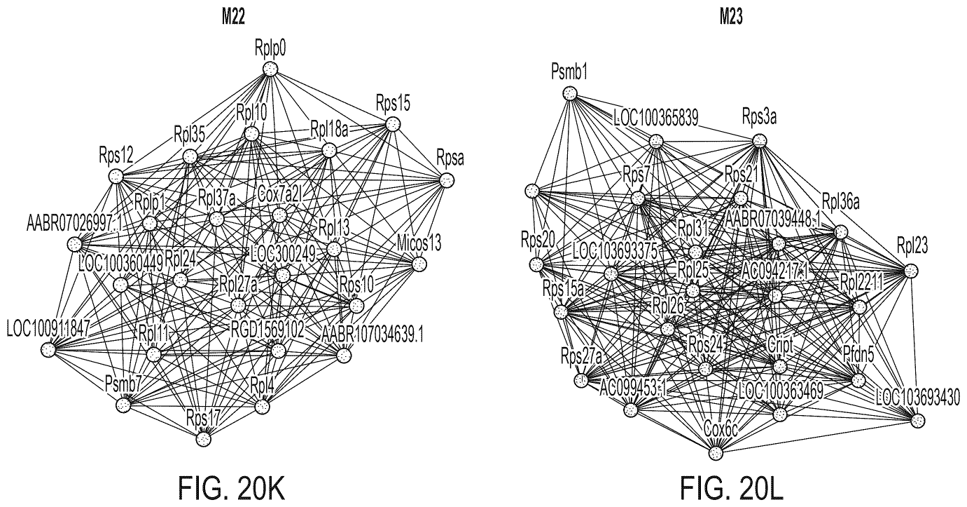

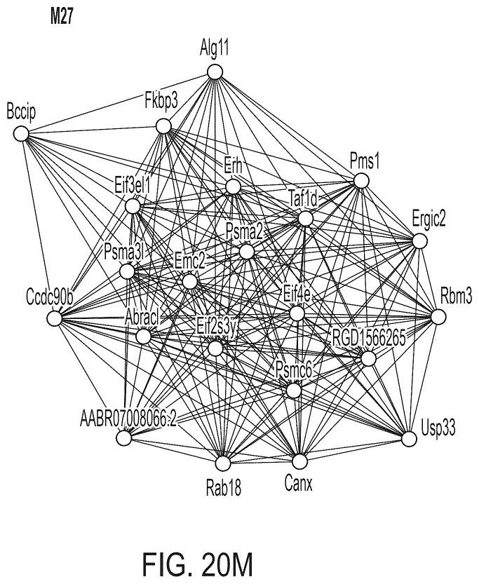

FIG. 20A illustrates another clustering dendrogram of genes with gene dissimilarity based on topological overlap on Day 7 post-treatment. FIG. 20B illustrates the number of genes per co-expression module detected on Day 7 post-treatment.

The co-expression analysis of the DGE results (as depicted in FIGS. 19A-19G and FIGS. 20A-20B) showed three modules that were significantly changed after vandefitemcel cell treatment/transplantation in comparison to the vehicle treatment group after stroke Day 3 (see FIGS. 19A-19G) and 10 modules on Day 7 post-treatment (see FIGS. 20A-20B).

FIG. 20C illustrates significant modules associated with the comparison stroke hMSC-SB623 group (or stroke vandefitemcel group) vs. the stroke vehicle group on blood collection Day 7 post-treatment. FIG. 20D illustrates the topmost relevant GO, KEGG, WP, and Reactome upregulated and downregulated pathways for the significant co-expression modules (modules 3, 10, 12, 13, 14, 18, 22, 23, and 27). Selected pathways have a minimum of 3 associated genes (intersect >=3).

FIG. 20E illustrates networks of the most connected genes (“eigengenes”) in module 3. FIG. 20F illustrates networks of the most connected genes (“eigengenes”) in module 10. FIG. 20G illustrates networks of the most connected genes (“eigengenes”) in module 12. FIG. 20H illustrates networks of the most connected genes (“eigengenes”) in module 13. FIG. 20I illustrates networks of the most connected genes (“eigengenes”) in module 14. FIG. 20J illustrates networks of the most connected genes (“eigengenes”) in module 18. FIG. 20K illustrates networks of the most connected genes (“eigengenes”) in module 22. FIG. 20L illustrates networks of the most connected genes (“eigengenes”) in module 23. FIG. 20M illustrates networks of the most connected genes (“eigengenes”) in module 27.

Cell type enrichment analysis showed that at the time of analysis stroke still strongly activated different immune cell populations even though it had been induced more than one month earlier.

FIG. 17G is a heatmap depicting cell-type specificity of enrichment signals. Downregulated (top rows) and upregulated (bottom rows) differentially expressed features show substantial enrichment for known blood cell type markers on Day 3 and Day 7 after treatment with vandefitemcel cells or vehicle one-month post-stroke. The shading indicates the −log₁₀ of the false discovery rate (FDR).

Only genes related to B cells were significantly downregulated in the stroke vehicle group compared to the sham vehicle group on both days. Genes associated with several different T cell populations were upregulated on both days (naïve T cells, CD4⁺CD25⁺ regulatory T cells, CD8⁺ T cells, and natural killer T (NKT) cells). CD4⁺ T cell genes were only upregulated on Day 3, whereas naive CD4⁺ and CD8⁺ T cell genes, as well as Th1 and Th17 and monocyte genes were upregulated only on Day 7 (see FIG. 17G). Vandefitemcel cell treatment/transplantation (in the comparison between the stroke vandefitemcel group and the stroke vehicle group) induced few significant changes in cell type enrichment. The only cell type upregulated by vandefitemcel cell treatment/transplantation were B cell genes on Day 7 post-treatment, whereas naïve T cell and CD8⁺ T cell genes were significantly down-regulated only on Day 3 after vandefitemcel cell treatment/transplantation compared to the stroke vehicle group.

This is the first time that such a wide range of effects of intracortical administration of vandefitemcel cells on gene expression in the peripheral blood has been shown. The most surprising and interesting observation is that vandefitemcel cell treatment/transplantation reverses part of the stroke-induced changes and affects targets that were previously described not only in the context of stroke but also other neurological disorders such as neurodegeneration.

### Vandefitemcel Transplantation Affects Protein Expression in Serum and Ameliorates Some Stroke-induced Changes

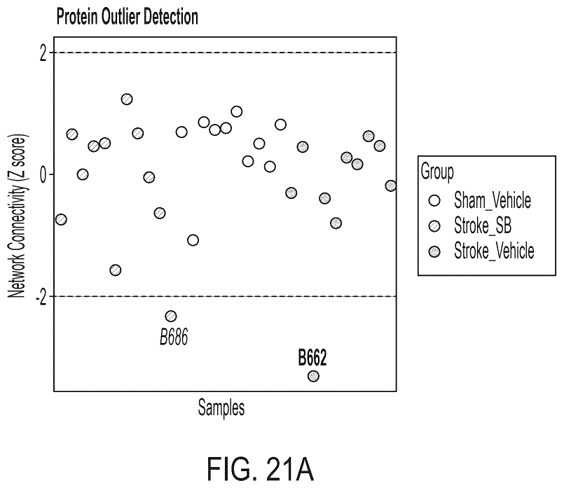

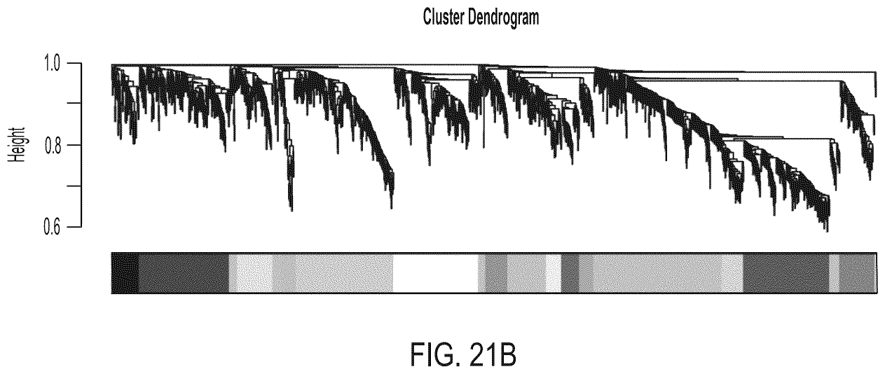

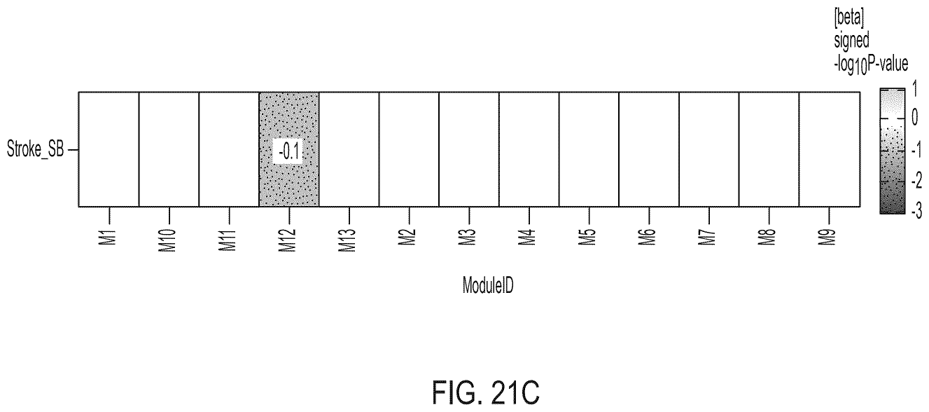

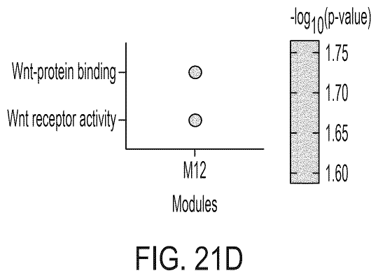

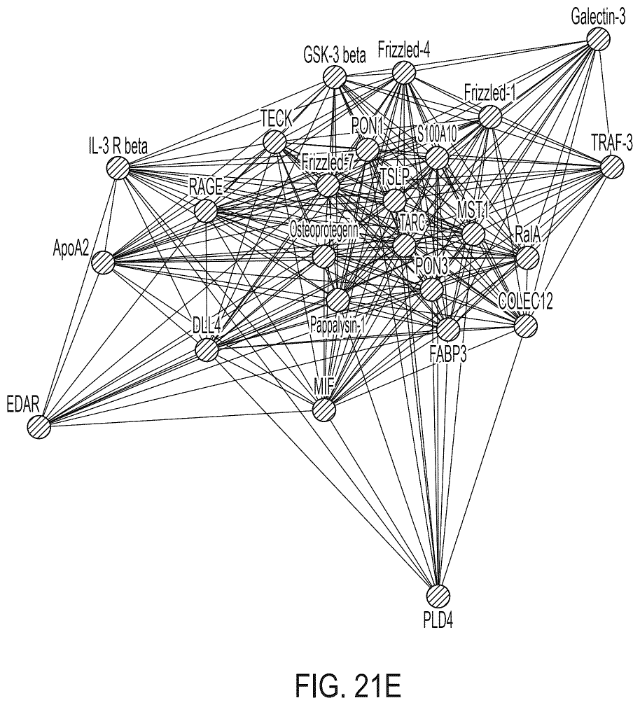

Proteomics analysis was done on serum samples collected on Day 7 post-treatment (see FIG. 21A for outlier analysis). Stroke had a considerable effect on serum protein levels—even more than a month after it had been induced.

FIG. 17H illustrates the total number of significantly downregulated or upregulated serum proteins on Day 7 after treatment with vandefitemcel cells or with the vehicle one-month post-stroke.

FIGS. 17I and 17J are volcano plots of differentially expressed proteins for the stroke vehicle group compared to the sham vehicle (group FIG. 17I) and the stroke vandefitemcel group compared to the stroke vehicle group (FIG. 17J) on Day 7 post-treatment. Downregulated genes are represented in darker shading and unregulated genes are represented in lighter shading.

In the stroke vandefitemcel group, a comparison to the stroke vehicle group showed five significantly up-regulated proteins and 25 significantly down-regulated proteins (see FIGS. 17H, 17I, and 17J).

FIG. 17K illustrates a list of “rescued” proteins that were upregulated or downregulated by stroke and upregulated or downregulated by vandefitemcel treatment. The color shading indicates the log₂ of the FC.

For five of these proteins, vandefitemcel cell treatment/transplantation reversed serum protein level changes that were induced by stroke: Filamin A (upregulated), Cathepsin L (downregulated), ApoE (downregulated), ARHGAP1 (downregulated), and TALDO1 (downregulated) (see FIG. 17K).

Cathepsin L has been described to play a role in brain tissue loss and inflammation after ischemic stroke, and Cathepsin L plasma levels are increased in the acute phase after ischemic stroke (Ma et al., 2022). Apart from its role in Alzheimer's disease, polymorphisms in the APOE gene have been found to be risk factors for ischemic stroke (Qiao et al., 2022), and APOE4 homozygosity has been shown to accelerate the development of dementia after stroke (Pendlebury et al., 2020). A co-expression analysis of the proteomics results found one protein module that was downregulated in the stroke vandefitemcel group compared to the stroke vehicle group. This module is associated with pathways for signaling receptor activity (which includes the following proteins COLEC12, GABRA4, LGALS3, CSF2RB, AGER, TGFBR1, FLT4) and more specifically also Wnt receptor activity (with proteins such as FZD1, FZD4, FZD7) (see FIGS. 21A-21E).

FIG. 21A is a plot with a sample outlier removal performed based on Z-scores of standardized network connectivity for serum samples collected on Day 7 after treatment with vandefitemcel, sham vehicle, or stroke vehicle which were administered one month post-stroke. FIG. 21B illustrates a clustering dendrogram of proteins with protein dissimilarity based on topological overlap.

FIG. 21C illustrates modules associated with the comparison of the stroke vandefitemcel group vs. the stroke vehicle group on blood collection Day 7 post-treatment. Module 12 is downregulated for the stroke vandefitemcel group. FIG. 21D illustrates the topmost relevant GO downregulated pathways for the significant co-expression module 12. Selected pathways have a minimum of three associated genes (intersect >=3). FIG. 21E illustrates a network of the most connected genes (“eigengenes”) in module 12.

These results show that intracortical/intracerebral treatment with vandefitemcel cells not only affects gene expression in the peripheral blood but also reverses some of the stroke-induced changes in serum proteins.

Gene Expression Changes in Blood Correlate with BDNF Brain Histology

One application of the blood analysis disclosed herein is to identify biomarkers in the blood that can reveal the effect of the cell transplants on the brain. To find such potential biomarkers, experiments were conducted for genes whose expression in the blood correlated with specific effects of the transplants, focusing on BDNF, since it was upregulated by the cell treatment/transplantation in different brain regions. To this end, all genes from blood samples obtained from the rats that were significantly upregulated or down-regulated after treatment with vandefitemcel were reviewed and their expression levels were correlated with the amount of BDNF staining in the brain of such rats. All rats and brain ROIs were included in this analysis.

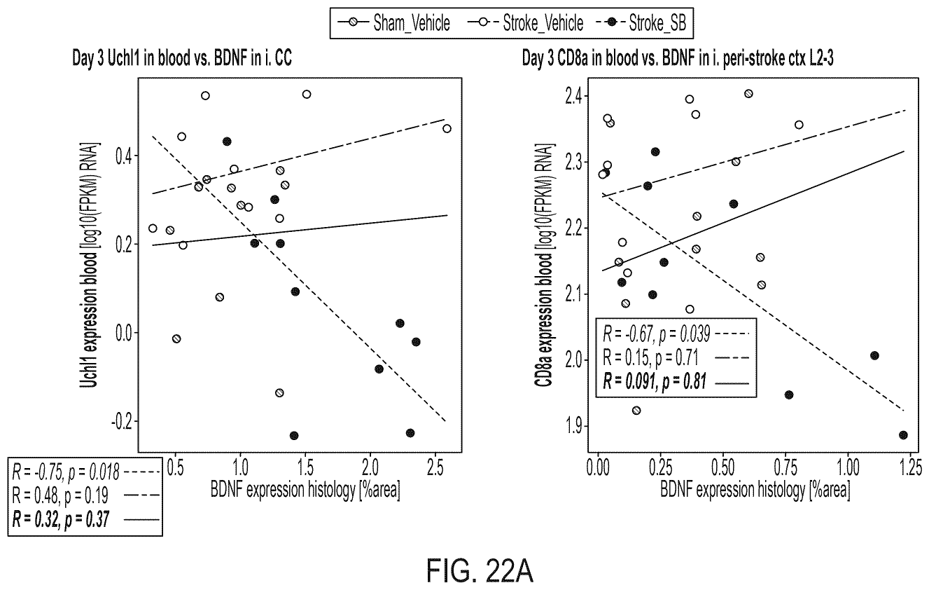

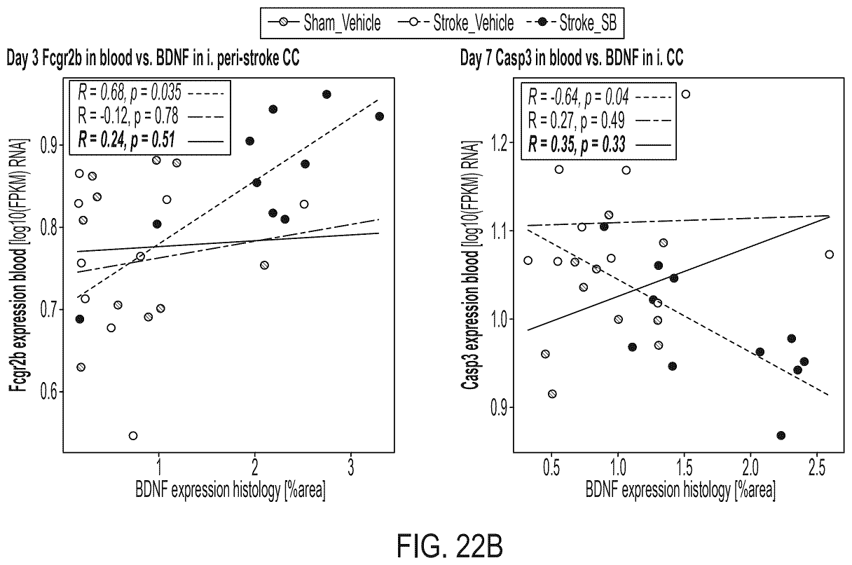

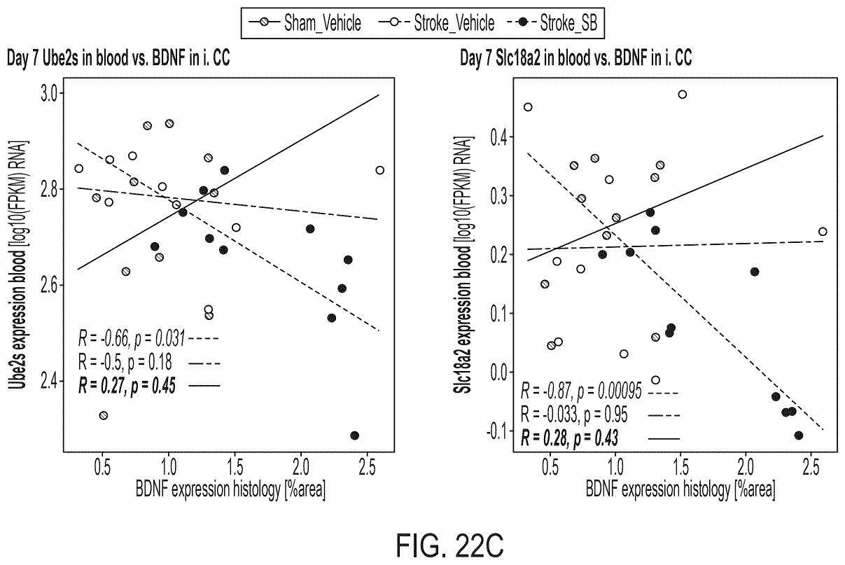

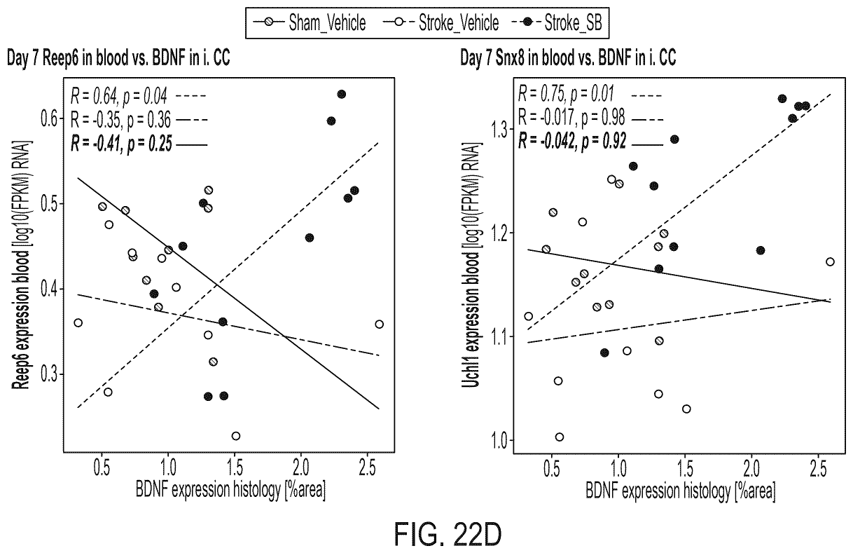

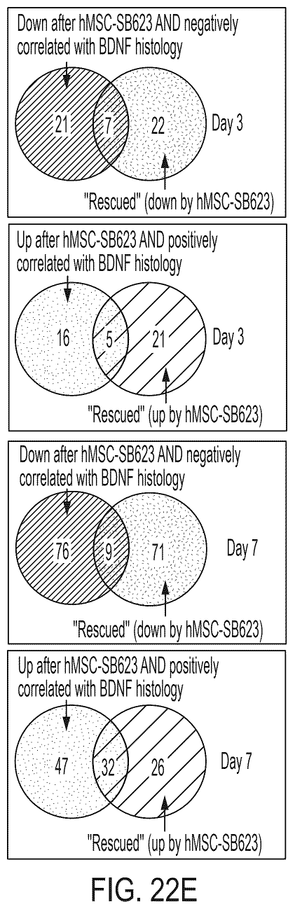

FIGS. 22A to 22D illustrate various examples of whole-blood genes that were differentially expressed in the stroke vandefitemcel group compared to the stroke vehicle group and significantly correlated (p<0.05, R>0.6; exact values indicated in the graphs) with BDNF expression (% area) in the brain on post-treatment Day 7 in at least one of the analyzed ROIs in the stroke vandefitemcel group. On Day 3 (FIGS. 22A and 22B) post-treatment, Uchl1 and CD8a were downregulated in blood in the stroke vandefitemcel group (compared to the stroke vehicle group) and negatively correlated with Day 7 BDNF brain histology the ipsilesional corpus callosum and peri-stroke cortex L2-3, respectively, in the stroke hMSC-SB623 group, whereas Fcgr2b was upregulated in blood and positively correlated with Day 7 BDNF brain histology in the peri-stroke corpus callosum. On Day 7 (FIGS. 22C and 22D), Casp3, Ube2s, and Slc18a2 genes were downregulated and correlated negatively with BDNF protein expression in the ipsilesional corpus callosum in the stroke vandefitemcel group, whereas Reep6 and Snx8 were upregulated and positively correlated with BDNF in the ipsilesional corpus callosum.

To make the interpretation of the results easier, only downregulated blood genes that were negatively correlated with BDNF expression in brain histology or upregulated blood genes that were positively correlated with BDNF in brain tissue (examples are shown in FIGS. 22A-22D) were selected.

In a second step, any of the vandefitemcel-modulated blood genes that significantly correlated with brain BDNF levels in the vandefitemcel group were also included in the list of “rescued” blood genes (see FIG. 17F) were also checked. “Rescued” refers to genes whose expression was modified in opposite directions by stroke and vandefitemcel cell treatment/transplantation in the blood. The results of this analysis are reported in FIG. 22E.

FIG. 22E are diagrams illustrating the overlap between the post-treatment Day 3 (top two images) and post-treatment Day 7 (bottom two images) downregulated/upregulated rescued whole-blood genes (genes that were upregulated/downregulated by stroke and downregulated by vandefitemcel treatment) and the whole-blood genes that were upregulated/downregulated by vandefitemcel cells (compared to vehicle treatment) on Day 3 or Day 7 and negatively/positively correlated with BDNF protein expression in the brain on Day 7 after treatment.

Having collected blood on Day 3 post-treatment, which was four days before brain tissue harvest, potential biomarkers in the blood that might precede vandefitemcel-mediated stimulation of BDNF expression in different brain regions were identified, although this would have to be confirmed in experiments with additional earlier time points for histology.

The most intriguing among these factors, Uchl1, CD8a, and Fcgr2b, with a strong correlation of their expression in the blood and brain BDNF levels in the vandefitemcel stroke group, that is not evident in the other two groups, are shown in FIGS. 22A-22D.

Uchl1, the gene for ubiquitin C-terminal hydrolase, was downregulated in whole-blood on Day 3 after treatment with vandefitemcel cells (compared to vehicle treatment) and negatively correlated with BDNF protein expression in multiple brain regions (for example, in the ipsilesional whole corpus callosum, FIGS. 22A-22D, and cortex, as well as in the peri-stroke regions of these two structures. UCHL1 is a protein that is expressed in neurons and is important for axonal function. It also plays a role in functional recovery from axonal injury and cerebral ischemia (Liu et al., 2019), and has been shown to have potential, that might even be correlated with functional outcome, as a blood biomarker for TBI as well as ischemic and hemorrhagic stroke (Ren et al., 2016; Yigit et al., 2017; Chen et al., 2021; Korley et al., 2022).

Interestingly, both CD8a (see FIGS. 22A-22D) and CD8b that were downregulated after vandefitemcel cell administration in blood collected on Day 3 post-treatment (with CD8b also being “rescued” and downregulated on post-treatment Day 7) also negatively correlated with BDNF expression in brain histology of the stroke vandefitemcel group.

On Day 3 post-treatment Fcgr2b RNA was upregulated in the blood as well as positively correlated with BDNF protein expression in multiple brain regions, for example, in the ipsilesional cortex and corpus callosum as well as the peri-stroke corpus callosum (see FIGS. 22A-22D). Fcgr2b is the gene for the inhibitory Fe gamma receptor IIB and is broadly expressed on leukocytes and regulates B cell function (Tanigaki et al., 2015). Fcgr2b has been identified as a hub gene in network analyses in two studies in rodent photothrombotic cortical stroke models investigating transcriptomic changes in the ischemic penumbra (Choi et al., 2019) and in the spinal cord (Kaiser et al., 2019). These results show that early after vandefitemcel treatment factors in the blood that are associated with the innate and adaptive immune system, and neuro-recovery not only after stroke but also after TBI, are modulated and correlate with brain BDNF levels four days later.

The most interesting blood factors that were strongly correlated with brain BDNF expression in the vandefitemcel stroke group on the same day as brain tissue collection are shown in FIGS. 22A-22D, and are as follows: On Day 7, Casp3, Ube2s, and Slc18a2 genes were downregulated and correlated negatively with BDNF protein expression in the ipsilesional corpus callosum in the stroke vandefitemcel group, whereas Reep6 and Snx8 were upregulated and positively correlated with BDNF in the ipsilesional corpus callosum (see FIGS. 22A-22D). Serum levels of caspase-3 (the protein encoded by Casp3) and one of its cleavage products, caspase-cleaved cytokeratin-18, have shown some promise as blood biomarkers in TBI, intracerebral hemorrhage, and cerebral ischemic stroke (Lorente et al., 2015; Glushakova et al., 2017; Sun et al., 2017; Lorente et al., 2019; Lorente et al., 2020). Ube2s, encoding ubiquitin-conjugating enzyme E2 S, UBE2S, has been identified as a hub gene in the peripheral blood of ischemic stroke patients (Chen and Wu, 2021; Zheng et al., 2022). Slc18a2 encodes vesicular monoamine transporter 2 (VMAT2) and is involved in dopamine and serotonin release, and a mutation in this gene is the cause of Brain Dopamine-Serotonin Vesicular Transport Disease (Rilstone et al., 2013).

VMAT2 dysfunction has also been implicated in Parkinson's Disease (Lohr and Miller, 2014), and a PET tracer targeting this protein for brain imaging has shown great promise for tracking Parkinson's disease progression (Beauchamp et al., 2023). A deficiency in Reep6 (which was upregulated after vandefitemcel cell treatment/transplantation in peripheral blood and positively correlated with BDNF expression in the brain) has been shown to lead to retinal degeneration because of disturbance of ER homeostasis and trafficking of proteins (Agrawal et al., 2017). Snx8 encodes for a sortin nexin, a class of proteins that are involved in different aspects of protein trafficking and endocytosis, and have been proposed as therapeutic targets for cardiovascular disorders (Yarmohammadi et al., 2022). To summarize, on Day 7 after intracortical vandefitemcel transplantation, not only factors with known connections with stroke and brain injury are changed in the blood and correlate with brain BDNF expression, but also factors with roles in neurodegenerative and cardiovascular disease.

For the serum proteins, only ANKRD9, downregulated in the serum, was negatively correlated with BDNF expression in brain histology of the vandefitemcel group, whereas only Ephrin-B2, upregulated in the serum, was positively correlated with brain BDNF levels (both proteins were not “rescued”) (see FIG. 22E). Interestingly, in a rat ischemic hindlimb ischemia model, treatment with vandefitemcel cells improved tissue perfusion and led, e.g., to a trend towards an increase of ephrin-B2 and higher EphB4 RNA in the treated muscle tissue (Maeda et al., 2021).

In summary, these correlations between factors in peripheral blood that were changed by vandefitemcel cell injection into the peri-stroke cortex and BDNF protein levels in the blood suggest that it is feasible to identify biomarkers in the peripheral blood of patients treated intracortically with vandefitemcel cells that are meaningful for functional recovery after ischemic stroke in the future. The following factors seem to be the most promising to follow up with: CD8a/b expression in T cells to measure the immunomodulatory effects of vandefitemcel that previously only had been shown in vitro (Dao et al., 2011); Fcgr2b because of its regulatory immune function (Tanigaki et al., 2015) and connection with CNS ischemia (Choi et al., 2019; Kaiser et al., 2019); Uchl1 because of its association with axonal function and neuro-recovery after TBI and cerebral stroke (Ren et al., 2016; Yigit et al., 2017; Liu et al., 2019; Chen et al., 2021; Korley et al., 2022); and Ube2s because of its role in ischemic stroke (Chen and Wu, 2021; Zheng et al., 2022).

Based on the experimental results discussed here, it is clear that vandefitemcel cell transplantation, even one month after a stroke, can normalize aberrant cortical excitability and induce, among other things, synaptic plasticity, oligodendrogenesis, and astrocytogenesis.

The experimental results also revealed: 1) increased levels of synaptic vesicles in the peri-stroke cortex, suggesting changes to synaptic activity, and 2) decreased ratio of PNNs between the peri-stroke cortex and the corresponding contralesional region. The observed increase in synaptic vesicles in the peri-stroke cortex indicates synaptogenesis or synaptic plasticity, both of which would alter synaptic signaling, which in turn could change overall cortical excitability. Similarly, MSCs or MSC-derived exosomes have been shown to stimulate synaptic plasticity in models of vascular dementia and ischemic stroke (Wang et al., 2019; Xin et al., 2013).

Vandefitemcel transplantation can also alter perineuronal nets (PNNs) and stimulate the expression of a GABA reuptake transporter. Additionally, the experimental results revealed that intracortical vandefitemcel treatment modulates stroke-induced changes in peripheral immune factors, some of which might be associated with its pro-regenerative effects such as an increase in brain BDNF levels. PNNs modulate neuronal excitability, in part by stabilizing synaptic connections; a transient loosening of PNNs has been associated with post-stroke synaptic remodeling (Dzyubenko et al., 2023).

Vandefitemcel cell transplants acted as disease modifiers, meaning these transplanted cells reversed the peri-stroke hyperexcitability. The efficacy of the vandefitemcel cell transplants was surprising for two reasons: only a small number of these transplanted cells survived (which is consistent with what others have described in Yasuhara et al., 2009; Tajiri et al., 2013; Kawauchi et al., 2022) and the transplant occurs after the cortical hyperexcitability has already been established and is presumably hard to modify.

Vandefitemcel cells are derived from mesenchymal stem/stromal cells (MSCs), which are renowned for remarkable “hit-and-run” therapeutic effects, long after their initial administration (Ng et al., 2015). This characteristic stems from their ability to modulate the microenvironment and influence various cellular processes. Despite a relatively low number of surviving cells (Toma et al., 2009), MSCs exert a sustained impact by producing extracellular vesicles (EVs) (Rani et al., 2015) and releasing a diverse array of bioactive molecules, including growth factors (Luo et al., 2012) and cytokines (Ranganath et al., 2012). Similar processes might be involved in the long-lasting treatment effects of vandefitemcel. Thus, vandefitemcel transplantation is a great tool to target disease-modifying mechanisms for chronic cortical network hyperexcitability.

Also, MBP, a component of myelin, was reduced by stroke in the peri-injury cortex, a region in which vandefitemcel treatment stimulated the proliferation of OPCs which may later differentiate into myelinating oligodendrocytes. Also, in regions of the contralesional hemisphere, such as the cortex, stroke and cell treatment/transplantation had opposite effects on MBP expression. These results suggest that vandefitemcel may correct cortical hyperexcitability, at least in part, by influencing the pathophysiological mechanisms of stroke that impact brain circuitry. Vandefitemcel cells correct cortical excitability by partly reversing alterations caused by the stroke injury (e.g., BDNF and MBP) but perhaps also by other compensatory pro-regenerative processes that are not necessarily affected by stroke. Mechanistically, it seems plausible that some of the observed neuro-regenerative processes, such as the increase in synaptic vesicles, and the myelin plasticity happen downstream of the vandefitemcel-mediated stimulation of endogenous BDNF production, since it has been previously demonstrated that BDNF stimulates synaptic plasticity and myelination (Berretta et al., 2014; Liu et al., 2020a).

In addition to their effect on the brain, the transcriptomic and proteomic blood analyses showed that intracortical transplantation of vandefitemcel cells into the peri-stroke cortex reversed the effects of stroke on peripheral blood factors that were not only immunomodulatory, such as CD8b, but also had associations with previously described stroke, TBI and/or neurodegenerative disease pathways, such as Cathepsin L (Ma et al., 2022), ApoE (Pendlebury et al., 2020; Qiao et al., 2022), Scarf1 (Wu et al., 2022), Dusp1 (Yang et al., 2022; Zhou et al., 2023).

Vandefitemcel likely release factors such as cytokines, growth factors, microRNAs, and extracellular vesicles, locally, and that these factors then affect immune cells in the blood. Vandefitemcel cells might also stimulate endogenous processes in the brain tissue that in turn, directly or indirectly, influence factors in the blood as a consequence of the active crosstalk between CNS and periphery which is also involved in recovery from cerebral stroke (reviewed for example in Liu et al., 2020b; Zhaolong et al., 2022).

The finding that part of the observed vandefitemcel-induced changes in blood factors significantly correlate with brain BDNF levels (see FIGS. 22A-22E), suggests that some of the identified changes in peripheral blood represent correlates of endogenous regenerative processes that are triggered by vandefitemcel cell injection. Promising factors seem to be, for example, the known stroke hub gene Ube2s (Chen and Wu, 2021; Zheng et al., 2022), or Uchl1, because of its involvement in neuro-recovery processes in the context of TBI and cerebral stroke (Ren et al., 2016; Yigit et al., 2017; Liu et al., 2019; Chen et al., 2021; Korley et al., 2022), and potential immunomodulatory effects on CD8⁺ T cells.

The experimental results show that vandefitemcel transplantation counteracts cortical hyperexcitability that develops after ischemic stroke and suggests that they exert protective effects on the brain by stimulating adaptive neural and myelin plasticity. Because cortical hyperexcitability is associated with poor outcomes after stroke in patients, vandefitemcel cell transplants acted as disease modifiers even when such cells were administered at a chronic time point by “reopening” the window of adaptive plasticity.

Methods of Treatment and Biomarker Panels Discovered/Developed in view of the Experimental Results

One unexpected discovery from the experiments and experimental results described herein is that the administration or implantation of vandefitemcel to a region of the brain of a subject can reduce chronic cortical hyperexcitability in the subject. As previously discussed, the vandefitemcel cells are cells descended from mesenchymal stem cells (MSCs) transiently-transfected by a polynucleotide encoding a Notch intracellular domain (NICD).

The vandefitemcel can treat the chronic cortical hyperexcitability by increasing production of gamma-aminobutyric acid (GABA) transporters 1 (GAT1) in the brain of the subject, increasing production of brain-derived neurotrophic factors (BDNF) in the brain of the subject, increasing production of doublecortin-positive (DCX⁺) neuronal progenitor cells (NPCs), increasing production of oligodendrocyte precursor cells (OPCs), increasing production of myelin basic protein (MBP), increasing production of glial fibrillary acidic protein-positive (GFAP⁺) astrocytes, increasing production of ionized calcium-binding adaptor molecule 1-positive (Iba1⁺) microglia in the brain of the subject, or a combination thereof.

The OPCs can be oligodendrocyte transcription factor 2-positive (Olig2+) OPCs or proliferating cell nuclear antigen-positive (PCNA+) OPCs.

The vandefitemcel can treat the chronic cortical hyperexcitability by increasing the production of GAT1 in at least one of a peri-stroke cortex, corpus callosum, somatosensory thalamus, internal capsule, ipsilesional hemisphere, and a contralesional hemisphere of the brain of the subject.

The production of MBP can be increased in a contralesional cortex of the subject in the brain of the subject.

The vandefitemcel can also treat the chronic cortical hyperexcitability by inducing synaptogenesis in a peri-stroke cortex of the brain of the subject.

The vandefitemcel can also treat the chronic cortical hyperexcitability by reducing glutamatergic synaptic vesicles in an ipsilesional region of a peri-stroke cortex of the brain compared to a contralesional region of the peri-stroke cortex.

The vandefitemcel can also treat the chronic cortical hyperexcitability by reducing perineuronal nets in an ipsilesional region of a peri-stroke cortex of the brain compared to a contralesional region of the peri-stroke cortex.

The vandefitemcel can be administered (parenterally) via intracerebral implantation. For example, administering the vandefitemcel can further include injecting the vandefitemcel at multiple sites within the region of the brain. The vandefitemcel can be administered stereotactically via a burr hole in a skull of the subject. The vandefitemcel can be administered in a peri-stroke cortex of the brain.

Administering the vandefitemcel can further include administering between about 1.0 million cells and about 10.0 million cells in total. For example, the amount of vandefitemcel cells administered can be approximately 2.5 million cells in total (or 2.5 million cells ±0.1 million cells), approximately 5.0 million cells in total (or 5.0 million cells ±0.1 million cells), or between about 2.5 million cells and about 5.0 million cells in total. The vandefitemcel can further be administered in accordance with administration procedures or protocols disclosed in U.S. Pat. No. 12,121,544, the content of which is incorporated herein by reference in its entirety.

Another unexpected discovery from the experiments and experimental results described herein is that the administration or implantation of vandefitemcel to a region of the brain of a subject can reverse stroke-induced changes in whole-blood gene expression. The changes can be induced by an ischemic stroke and the vandefitemcel can be administered thirty days or more after the ischemic stroke.

The vandefitemcel can reverse stroke-induced changes in whole-blood gene expression by downregulating CD8a gene expression, downregulating CD8b gene expression, downregulating Uchl1 gene expression, downregulating Casp3 gene expression, downregulating Ube2s gene expression, downregulating Slc18a2 gene expression, upregulating Fcgr2b gene expression, or a combination thereof.

Moreover, the vandefitemcel can reverse stroke-induced changes in whole-blood gene expression by downregulating Scarf1 gene expression, downregulating Dusp1 gene expression, downregulating Csnk2a1 gene expression, or a combination thereof.

Yet another unexpected discovery from the experiments and experimental results described herein is that the administration or implantation of vandefitemcel to a region of the brain of a subject can reverse stroke-induced changes in serum protein expression. The changes can be induced by an ischemic stroke and the vandefitemcel can be administered thirty days or more after the ischemic stroke.

The vandefitemcel can reverse stroke-induced changes in serum protein expression by upregulating Filamin A protein expression, downregulating Cathepsin L protein expression, downregulating ApoE protein expression, downregulating ARHGAP1 protein expression, downregulating TALDO1 protein expression, or a combination thereof.

A further unexpected discovery from the experiments and experimental results described herein is that the administration or implantation of vandefitemcel to a region of the brain of a subject can induce endogenous production of BDNFs in the brain of the subject. For example, the endogenous production of BDNFs can be increased in a peri-stroke cortex, a corpus callosum, a somatosensory thalamus, an internal capsule, an ipsilesional hemisphere, and/or a contralesional hemisphere of the brain of the subject.

One technical problem faced by the applicant is how to assess the efficacy of stem cell treatments (e.g., vandefitemcel treatment) for stroke or chronic conditions caused by stroke when the stem cell treatment is administered intracerebrally. One technical solution discovered and developed by the applicant is a blood-based biomarker panel for assessing the efficacy of a stem cell treatment when the stem cells are administered intracerebrally to a subject.

The blood-based biomarker panel can include testing for the following biomarker genes: CD8a, CD8b, Uchl1, Casp3, Ube2s, Slc18a2, Fcgr2b, Scarf1, Dusp1, Csnk2al, or a combination thereof. The blood-based biomarker panel can also include testing for the following protein biomarkers: Cathepsin L, ApoE, ARHGAP1, TALDO1, or a combination thereof.

In other variations, the blood-based biomarker panel can also include testing for the following biomarker gens: U6, Stx 17, Hsd17b14, AABR07008608.1, RGD1309651, AABR07027451.1, Micall2, AABR07051532.2, Klhdc9, AABR07019663.1, Pds5a, RGD1309779, Runx3, S100pbp, Kctd6, Lipe, AABRO7051715.1, LOC690466, Fbin1, Timm17b on Day 3 and Ndufa1, Timm17b, Mif, RT1-DMb, Mff, Mir22, LOC103689947, LOC691427, RT1-T-24-1, RGD1310166, Ticd4, Tpm2, AABR07044362.1, AABR07072025.1, RT1-CE7, Pacrgl, RT1-DMa, Hmgn1, Fbin1, or a combination thereof.

For example, a method of assessing an efficacy of a treatment for a chronic condition caused by stroke can comprise obtaining a first sample of blood (a pre-treatment sample) from a subject before the treatment; administering vandefitemcel to a region of the brain of a subject; obtaining a second sample of blood (a post-treatment sample) from a subject at least three days (or at least seven days) after administering the vandefitemcel; comparing gene expression levels between the second sample of blood and the first sample of blood for at least one of the following biomarker genes: CD8a, CD8b, Uchl1, Casp3, Ube2s, and Slc18a2; and classifying or categorizing the vandefitemcel administration as effective in ameliorating or improving the chronic condition caused by the stroke when at least one of the CD8a gene, the CD8b gene, the Uchl1 gene, the Casp3 gene, the Ube2s gene, and the Slc18a2 gene in the second sample of blood (the post-treatment sample) is downregulated relative to the first sample of blood (the pre-treatment sample).

The method can further comprise comparing a gene expression level between the second sample of blood (the post-treatment sample) and the first sample of blood (the pre-treatment sample) for the biomarker gene Fcgr2b and classifying or categorizing the vandefitemcel administration as effective in ameliorating or improving the chronic condition caused by the stroke when the biomarker gene Fcgr2b in the second sample of blood (the post-treatment sample) is upregulated relative to the first sample of blood (the pre-treatment sample).

The method can also comprise comparing gene expression levels between the second sample of blood (the post-treatment sample) and the first sample of blood (the pre-treatment sample) for at least one of the following biomarker genes: Scarf1, Dusp1, and Csnk2a1 and classifying the vandefitemcel administration as effective in ameliorating or improving the chronic condition caused by the stroke when at least one of the Scarf1 gene, the Dusp1 gene, and the Csnk2a1 gene in the second sample of blood (the post-treatment sample) is downregulated relative to the first sample of blood (the pre-treatment sample).

The method can further comprise comparing protein expression levels between the second sample of blood (the post-treatment sample) and the first sample of blood (the pre-treatment sample) for at least one of the following protein biomarkers: Cathepsin L, ApoE, ARHGAP1, and TALDO1 and classifying the vandefitemcel administration as effective in ameliorating or improving the chronic condition caused by the stroke when at least one of the Cathepsin L protein biomarker, the ApoE protein biomarker, the ARHGAP1 protein biomarker, and the TALDO1 protein biomarker in the second sample of blood (the post-treatment sample) is downregulated relative to the first sample of blood (the pre-treatment sample).

The method can also comprise comparing a protein expression level between the second sample of blood (the post-treatment sample) and the first sample of blood (the pre-treatment sample) for the protein biomarker Filamin A and classifying the vandefitemcel administration as effective in ameliorating or improving the chronic condition caused by the stroke when the protein biomarker Filamin A in the second sample of blood (the post-treatment sample) is upregulated relative to the first sample of blood (the pre-treatment sample).

The stroke can be ischemic stroke and the vandefitemcel can be administered thirty days or more after the ischemic stroke.

### Composition, Formulations, and Kits

Another unexpected discovery from the experiments and experimental results described herein is that a composition comprising vandefitemcel can treat chronic cortical hyperexcitability. The composition can comprise vandefitemcel in an amount between about 1.0 million cells and about 10.0 million cells (e.g., about 2.5 million cells, about 5.0 million cells, or between about 2.5 million cells and 5.0 million cells).

For example, the composition can comprise a 0.3 mL cell suspension with a cell concentration of approximately 8.5*10⁶ cells/mL. Also, for example, the composition can comprise a 0.3 mL cell suspension with a cell concentration of approximately 17.0*10⁶ cells/mL.

The composition comprising the vandefitemcel can be administered or implanted within a region of the brain of a subject.

The vandefitemcel can be produced by modifying mesenchymal stem cells derived from human bone marrow. The composition can also comprise one or more pharmaceutically acceptable excipients. The vandefitemcel can be made by a process including providing a culture of mesenchymal stem cells, contacting the culture of mesenchymal stem cells with a polynucleotide encoding a Notch intracellular domain (NICD) (the polynucleotide does not encode a full-length Notch protein), selecting cells that include the polynucleotide, and further culturing the selected cells in the absence of selection for the polynucleotide. The mesenchymal stem cells can be transiently-transfected with a plasmid vector including the polynucleotide encoding the NICD. The composition can also comprise some mesenchymal stem cells not transiently-transfected by the polynucleotide.

The composition can treat the chronic cortical hyperexcitability by increasing the production of gamma-aminobutyric acid (GABA) transporters 1 (GAT1) in the brain of a subject, increasing the production of brain-derived neurotrophic factors (BDNF) in the brain of the subject, or a combination thereof.

The composition can treat chronic cortical hyperexcitability by increasing the production of GAT1 in a peri-stroke cortex or ischemic penumbra of the brain of the subject. The composition can also treat chronic cortical hyperexcitability by increasing the production of BDNF in at least one of a peri-stroke cortex (or ischemic penumbra), a corpus callosum, a somatosensory thalamus, an internal capsule, a contralesional hemisphere, and an ipsilesional hemisphere of the brain of the subject.

The composition can treat chronic cortical hyperexcitability by increasing the production of doublecortin-positive (DCX⁺) neuronal progenitor cells (NPCs) in the brain of the subject.

The composition can also the chronic cortical hyperexcitability by increasing the production of oligodendrocyte precursor cells (OPCs). For example, the OPCs can be oligodendrocyte transcription factor 2-positive (Olig2⁺) OPCs, proliferating cell nuclear antigen-positive (PCNA⁺) OPCs, or a combination thereof.

The composition can treat chronic cortical hyperexcitability by increasing the production of myelin basic protein (MBP) in a contralesional cortex of the subject.

The composition can also treat chronic cortical hyperexcitability by increasing the production of glial fibrillary acidic protein-positive (GFAP⁺) astrocytes in the brain of the subject.

The composition can treat chronic cortical hyperexcitability by increasing the production of ionized calcium-binding adaptor molecule 1-positive (Iba1⁺) microglia in the brain of the subject.

The composition can also treat chronic cortical hyperexcitability by inducing synaptogenesis in a peri-stroke cortex of the brain of the subject.

The composition can also treat chronic cortical hyperexcitability by reducing glutamatergic synaptic vesicles in an ipsilesional region of a peri-stroke cortex of the brain compared to a contralesional region of the peri-stroke cortex.

The composition can also treat the chronic cortical hyperexcitability by reducing perineuronal nets (PNNs) in an ipsilesional region of a peri-stroke cortex of the brain compared to a contralesional region of the peri-stroke cortex.

Another unexpected discovery from the experiments and experimental results described herein is that a composition comprising vandefitemcel can treat chronic cortical hyperexcitability by reversing stroke-induced changes in whole-blood gene expression. The composition, comprising the vandefitemcel, can reverse the stroke-induced changes in whole-blood gene expression by downregulating CD8a gene expression, downregulating CD8b gene expression, downregulating Uchl1 gene expression, downregulating Casp3 gene expression, downregulating Ube2s gene expression, downregulating Slc18a2 gene expression, upregulating Fcgr2b gene expression, or a combination thereof. The composition comprising the vandefitemcel can be administered or implanted within a region of the brain of a subject.

The composition can also reverse stroke-induced changes in whole-blood gene expression by downregulating Scarf1 gene expression.

The composition can reverse stroke-induced changes in whole-blood gene expression by downregulating Dusp1 gene expression.

The composition can reverse stroke-induced changes in whole-blood gene expression by downregulating Csnk2a1 gene expression.

Yet another unexpected discovery from the experiments and experimental results described herein is that a composition comprising vandefitemcel can treat chronic cortical hyperexcitability by reversing stroke-induced changes in serum protein expression. The composition can reverse the stroke-induced changes in serum protein expression by upregulating Filamin A protein expression, downregulating Cathepsin L protein expression, downregulating ApoE protein expression, downregulating ARHGAP1 protein expression, and downregulating TALDO1 protein expression. The composition comprising the vandefitemcel can be administered or implanted within a region of the brain of a subject.

An additional unexpected discovery from the experiments and experimental results described herein is that a composition comprising vandefitemcel can induce endogenous production of BDNFs in a brain of a subject. The composition comprising the vandefitemcel can be administered or implanted within a region of the brain of a subject.

The vandefitemcel can be suspended in a sterile isotonic crystalloid solution.

The one or more pharmaceutically acceptable excipients can comprise at least one of buffers, proteins, stabilizers, and preservatives. The one or more pharmaceutically acceptable excipients can also comprise carriers or diluents to form the cell suspension.

The pharmaceutically acceptable carrier can be a physiologically compatible carrier for implantation. As used herein, the term “physiologically compatible carrier” can refer to a carrier that is compatible with the other ingredients of the formulation and not deleterious to the recipient thereof. Examples of suitable carriers or diluents include cell culture medium (e.g., Eagle's minimal essential medium), phosphate buffered saline, Hank's balanced salt solution+/−glucose (HBSS), and multiple electrolyte solutions. The pharmaceutically acceptable carrier or diluent can also be or comprise a sterile isotonic crystalloid solution such as Plasma-Lyte™ A (Baxter Healthcare Corporation).

The composition can comprise the vandefitemcel packaged in a sealed vial. In some instances, the sealed vial can comprise 0.3 mL of the cell suspension with a cell concentration of approximately 8.5*10⁶ cells/mL. Alternatively, the sealed vial can comprise 0.3 mL of the cell suspension with a cell concentration of approximately 17.0*10⁶ cells/mL.

Also disclosed are other examples of materials which can serve as pharmaceutically-acceptable carriers or excipients comprising: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances. Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the composition.

Other examples of pharmaceutically-acceptable carriers or excipients include substances that stimulate angiogenesis (“pro-angiogenic agents”). In some instances, the pro-angiogenic agent can be a protein (e.g., fibroblast growth factor, platelet-derived growth factor, transforming growth factor alpha, hepatocyte growth factor, vascular endothelial growth factor, sonic hedgehog, MAGP-2, HIF-1, PR-39, RTEF-1, c-Myc, TFII, Egr-1, ETS-1) or a nucleic acid encoding such a protein. See, for example, Vincent et al. (2007) Gene Therapy 14:781-789. In other instances, the pro-angiogenic agent can be a small RNA molecule (e.g., siRNA, shRNA, microRNA) or a ribozyme that targets a nucleic acid encoding an inhibitor of angiogenesis. Moreover, the pro-angiogenic agent can be a triplex-forming nucleic acid that binds to DNA sequences regulating the expression of a protein that inhibits angiogenesis, so as to block transcription of the gene encoding the protein.

Exemplary formulations comprise those suitable for parenteral administration, e.g., intrapulmonary, intra-arterial, intra-ocular, intra-cranial, sub-meningeal, or subcutaneous administration, including formulations encapsulated in micelles, liposomes or drug-release capsules (active agents incorporated within a biocompatible coating designed for slow-release); ingestible formulations; formulations for topical use, such as eye drops, creams, ointments, and gels; and other formulations such as inhalants, aerosols and sprays. The dosage of the compositions of the disclosure can vary according to the extent and severity of the need for treatment, the activity of the administered composition, the general health of the subject, and other considerations well known to the skilled artisan.

In additional embodiments, the compositions described herein can also be delivered locally. Localized delivery allows for the delivery of the composition non-systemically, thereby reducing the body burden of the composition as compared to systemic delivery. Such local delivery can be achieved, for example, through the use of various medically implanted devices including, but not limited to, stents and catheters, or can be achieved by inhalation, injection or surgery. Methods for coating, implanting, embedding, and otherwise attaching desired agents to medical devices such as stents and catheters are established in the art and contemplated herein.

Another aspect of the present disclosure relates to kits for carrying out the administration of the cells to a subject. For example, the kit can comprise the composition of cells, formulated as appropriate (e.g., in a pharmaceutical carrier), in one or more separate pharmaceutical preparations.

Compositions comprising vandefitemcel can be used in combination with other compositions comprising substances that stimulate angiogenesis (“pro-angiogenic agents”). The compositions can be administered sequentially in any order or concurrently. Accordingly, therapeutic compositions as disclosed herein can contain both vandefitemcel and a pro-angiogenic agent. In additional embodiments, separate therapeutic compositions, one comprising vandefitemcel and the other comprising a pro-angiogenic agent, can be administered to the subject, either separately or together.

The pro-angiogenic agent can be a transcription factor that activates expression of a pro-angiogenic molecule (e.g., protein). Naturally-occurring transcription factors (such as, for example, HIF-1alpha) that regulate the expression of pro-angiogenic proteins, are known. In addition, synthetic transcriptional regulatory proteins can be constructed by genetic engineering. For example, methods for the design of zinc finger DNA-binding domains that bind to a sequence of interest, and methods for the fusion of such zinc finger DNA-binding domains to transcriptional activation and repression domains, have been described. See, for example, U.S. Pat. Nos. 6,534,261: 6,607,882; 6,785,613; 6,794,136; 6,824,978; 6,979,539; 7,013,219: 7,177,766; 7,220,719; and 7,788,044. These methods can be used to synthesize non-naturally-occurring proteins that activate transcription of any gene encoding a pro-angiogenic protein. In addition, synthetic zinc finger transcriptional activators of the vascular endothelial growth factor (VEGF) gene have been described. See, e.g., U.S. Pat. Nos. 7,026,462; 7,067,317; 7,560,440: 7,605,140; and 8,071,564. Accordingly, a non-naturally-occurring (i.e., synthetic) zinc finger protein that activates transcription of the VEGF gene can be used, in combination with vandefitemcel, for augmenting angiogenesis, e.g., in the treatment of stroke. Furthermore, a natural or synthetic transcriptional regulatory protein (e.g., a synthetic zinc finger transcriptional regulatory protein) that inhibits transcription of an anti-angiogenic molecule can also be used as a pro-angiogenic agent.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of,” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having,” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth but is intended to cover alternatives, modifications, and equivalents of the variations or embodiments described herein. Further, the scope of the disclosure fully encompasses other variations or embodiments that may become obvious to those skilled in the art in view of this disclosure.

### REFERENCES

Agrawal, S. A., Burgoyne, T., Eblimit, A., Bellingham, J., Parfitt, D. A., Lane, A., Nichols, R., Asomugha, C., Hayes, M. J., Munro, P. M., Xu, M., Wang, K., Futter, C. E., Li, Y., Chen, R., Cheetham, M. E., 2017. REEP6 deficiency leads to retinal degeneration through disruption of ER homeostasis and protein trafficking. Hum. Mol. Genet. 26, 2667-2677. https://doi.org/10.1093/hmg/ddx149
Aizenman, C. D., Kirkwood, A., Bear, M. F., 1996. A current source density analysis of evoked responses in slices of adult rat visual cortex: implications for the regulation of long-term potentiation. Cereb. Cortex N. Y. N 1991 6, 751-758. https://doi.org/10.1093/cercor/6.6.751
Aizman, I., Tate, C. C., McGrogan, M., Case, C. C., 2009. Extracellular matrix produced by bone marrow stromal cells and by their derivative, SB623 cells, supports neural cell growth. J. Neurosci. Res. 87, 3198-3206. https://doi.org/10.1002/jnr.22146
Aizman, I., Tirumalashetty, B. J., McGrogan, M., Case, C. C., 2014. Comparison of the neuropoietic activity of gene-modified versus parental mesenchymal stromal cells and the identification of soluble and extracellular matrix-related neuropoietic mediators. Stem Cell Res. Ther. 5, 29. https://doi.org/10.1186/scrt418
Artavanis-Tsakonas, Spyros, Kenji Matsuno, and Mark E. Fortini. Notch signaling. Science 268.5208 (1995): 225-232.
Bardy, C., van den Hurk, M., Eames, T., Marchand, C., Hernandez, R. V., Kellogg, M., Gorris, M., Galet, B., Palomares, V., Brown, J., Bang, A. G., Mertens, J., Bohnke, L., Boyer, L., Simon, S., Gage, F. H., 2015. Neuronal medium that supports basic synaptic functions and activity of human neurons in vitro. Proc Natl Acad Sci USA 112, E2725-2734.
Bazargani, N., Attwell, D., 2016. Astrocyte calcium signaling: the third wave. Nat. Neurosci. 19, 182-189. https://doi.org/10.1038/nn.4201
Beauchamp, L. C., Dore, V., Villemagne, V. L., Xu, S., Finkelstein, D., Barnham, K. J., Rowe, C., 2023. Using 18F-AV-133 VMAT2 PET Imaging to Monitor Progressive Nigrostriatal Degeneration in Parkinson Disease. Neurology 101, e2314-e2324. https://doi.org/10.1212/WNL.0000000000207748
Berretta, A., Tzeng, Y.-C., Clarkson, A. N., 2014. Post-stroke recovery: the role of activity-dependent release of brain-derived neurotrophic factor. Expert Rev. Neurother. 14, 1335-1344. https://doi.org/10.1586/14737175.2014.969242
Bestmann, S., Swayne, O., Blankenburg, F., Ruff, C. C., Teo, J., Weiskopf, N., Driver, J., Rothwell, J. C., Ward, N. S., 2010. The role of contralesional dorsal premotor cortex after stroke as studied with concurrent TMS-fMRI. J. Neurosci. Off. J. Soc. Neurosci. 30, 11926-11937. https://doi.org/10.1523/JNEUROSCI.5642-09.2010
Bhatt, M., Gauthier-Manuel, L., Lazzarin, E., Zerlotti, R., Ziegler, C., Bazzone, A., Stockner, T., Bossi, E., 2023. A comparative review on the well-studied GAT1 and the understudied BGT-1 in the brain. Front. Physiol. 14.
Biose, I. J., Chastain, W. H., Wang, H., Ouvrier, B., & Bix, G. J. (2022). Optimizing intraluminal monofilament model of ischemic stroke in middle-aged Sprague-Dawley rats. BMC neuroscience, 23(1), 75.
Borgo, C., D'Amore, C., Sarno, S., Salvi, M., Ruzzene, M., 2021. Protein kinase CK2: a potential therapeutic target for diverse human diseases. Signal Transduct. Target. Ther. 6, 183. https://doi.org/10.1038/s41392-021-00567-7
Brent, R., et al. Current Protocols in Molecular Biology (Janssen, K., ed) Vol. 1. (1994).
Buzsáki, G., Anastassiou, C. A., Koch, C., 2012. The origin of extracellular fields and currents--EEG, ECoG, LFP and spikes. Nat. Rev. Neurosci. 13, 407-420. https://doi.org/10.1038/nrn3241
Campagnoli, Cesare, et al. Identification of mesenchymal stem/progenitor cells in human first-trimester fetal blood, liver, and bone marrow. Blood, The Journal of the American Society of Hematology 98.8 (2001): 2396-2402.
Campero-Romero, A. N., Real, F. H., Santana-Martinez, R. A., Molina-Villa, T., Aranda, C., Ríos-Castro, E., Tovar-Y-Romo, L. B., 2023. Extracellular vesicles from neural progenitor cells promote functional recovery after stroke in mice with pharmacological inhibition of neurogenesis. Cell Death Discov. 9, 272. https://doi.org/10.1038/s41420-023-01561-4
Ceanga, M., Dahab, M., Witte, O. W., Keiner, S., 2021. Adult Neurogenesis and Stroke: A Tale of Two Neurogenic Niches. Front. Neurosci. 15, 700297. https://doi.org/10.3389/fnins.2021.700297
Chen, C.-H., Chu, H.-J., Hwang, Y.-T., Lin, Y.-H., Lee, C.-W., Tang, S.-C., Jeng, J.-S., 2021. Plasma neurofilament light chain level predicts outcomes in stroke patients receiving endovascular thrombectomy. J. Neuroinflammation 18, 195. https://doi.org/10.1186/s12974-021-02254-4
Chen, X., Wu, D., 2021. Comprehensive Analysis of Hub Genes Associated With Competing Endogenous RNA Networks in Stroke Using Bioinformatics Analysis. Front. Genet. 12, 779923. https://doi.org/10.3389/fgene.2021.779923
Cho, F. S., Clemente, A., Holden, S., Paz, J. T., 2017. Chapter 19—Thalamic Models of Seizures In Vitro, in: Pitkanen, A., Buckmaster, P. S., Galanopoulou, A. S., Moshé, S. L. (Eds.), Models of Seizures and Epilepsy (Second Edition). Academic Press, pp. 273-284. https://doi.org/10.1016/B978-O-12-804066-9.00019-5
Choi, I.-A., Yun, J. H., Kim, J.-H., Kim, H. Y., Choi, D.-H., Lee, J., 2019. Sequential Transcriptome Changes in the Penumbra after Ischemic Stroke. Int. J. Mol. Sci. 20, 6349. https://doi.org/10.3390/ijms20246349
Cruikshank, S. J., Rose, H. J., Metherate, R., 2002. Auditory thalamocortical synaptic transmission in vitro. J. Neurophysiol. 87, 361-384. https://doi.org/10.1152/jn.00549.2001
Dao, M., Tate, C. C., McGrogan, M., Case, C. C., 2013. Comparing the angiogenic potency of naïve marrow stromal cells and Notch-transfected marrow stromal cells. J. Transl. Med. 11, 81. https://doi.org/10.1186/1479-5876-11-81
Dao, M. A., Tate, C. C., Aizman, I., McGrogan, M., Case, C. C., 2011. Comparing the immunosuppressive potency of naïve marrow stromal cells and Notch-transfected marrow stromal cells. J. Neuroinflammation 8, 133. https://doi.org/10.1186/1742-2094-8-133
de Curtis, M., Garbelli, R., Uva, L., 2021. A hypothesis for the role of axon demyelination in seizure generation. Epilepsia 62, 583-595. https://doi.org/10.1111/epi.16824
Dezawa, Mari, et al. Sciatic nerve regeneration in rats induced by transplantation of in vitro differentiated bone—marrow stromal cells. European Journal of Neuroscience 14.11 (2001):1771-1776.
Dezawa, Mari, et al. Specific induction of neuronal cells from bone marrow stromal cells and application for autologous transplantation. The Journal of clinical investigation 113.12 (2004):1701-1710.
Dhamoon, M. S., Longstreth, W. T., Bartz, T. M., Kaplan, R. C., Elkind, M. S. V., 2017.
Disability trajectories before and after stroke and myocardial infarction: the Cardiovascular Health Study. JAMA Neurol. 74, 1439-1445. https://doi.org/10.1001/jamaneurol.2017.2802
Dolgalev, Igor, 2021. Unbiased Single-Cell Transcriptomic Data Cell Type Identification [WWW Document]. URL https://igordot.github.io/clustermole/(accessed 11.29.23).
Durinck, S., Moreau, Y., Kasprzyk, A., Davis, S., De Moor, B., Brazma, A., Huber, W., 2005. BioMart and Bioconductor: a powerful link between biological databases and microarray data analysis. Bioinforma. Oxf. Engl. 21, 3439-3440. https://doi.org/10.1093/bioinformatics/bti525
Dzyubenko, E., Willig, K. I., Yin, D., Sardari, M., Tokmak, E., Labus, P., Schmermund, B., Hermann, D. M., 2023. Structural changes in perineuronal nets and their perforating GABAergic synapses precede motor coordination recovery post stroke. J. Biomed. Sci. 30, 76. https://doi.org/10.1186/s12929-023-00971-x
Ehebauer, Matthias, Penelope Hayward, and Alfonso Martinez-Arias. Notch signaling pathway. Science's STKE 2006.364 (2006): cm7-cm7.
Einevoll, G. T., Kayser, C., Logothetis, N. K., Panzeri, S., 2013. Modelling and analysis of local field potentials for studying the function of cortical circuits. Nat. Rev. Neurosci. 14, 770-785. https://doi.org/10.1038/nrn3599
Erices, Alejandro, Paulette Conget, and Jos6 J. Minguell. Mesenchymal progenitor cells in human umbilical cord blood. British journal of haematology 109.1 (2000): 235-242.
Fang, J., Wang, Z., Miao, C.-Y., 2023. Angiogenesis after ischemic stroke. Acta Pharmacol. Sin. 44, 1305-1321. https://doi.org/10.1038/s41401-023-01061-2
Fang, L.-P., Bai, X., 2023. Oligodendrocyte precursor cells: the multitaskers in the brain. Pflugers Arch. 475, 1035-1044. https://doi.org/10.1007/s00424-023-02837-5
Ferguson, B., Glick, C., Huguenard, J. R., 2023. Prefrontal PV interneurons facilitate attention and are linked to attentional dysfunction in a mouse model of absence epilepsy. eLife 12, e78349. https://doi.org/10.7554/eLife.78349
Fisher, Marc, and Birgul Bastan. Identifying and utilizing the ischemic penumbra. Neurology 79.13_supplement_1 (2012): S79-S85.
Fletcher, J. L., Murray, S. S., Xiao, J., 2018. Brain-Derived Neurotrophic Factor in Central Nervous System Myelination: A New Mechanism to Promote Myelin Plasticity and Repair. Int. J. Mol. Sci. 19, 4131. https://doi.org/10.3390/ijms19124131
Freeman, J. A., Nicholson, C., 1975. Experimental optimization of current source-density technique for anuran cerebellum. J. Neurophysiol. 38, 369-382. https://doi.org/10.1152/jn.1975.38.2.369
Frerking, M., Malenka, R. C., Nicoll, R. A., 1998. Brain-derived neurotrophic factor (BDNF) modulates inhibitory, but not excitatory, transmission in the CA1 region of the hippocampus. J. Neurophysiol. 80, 3383-3386. https://doi.org/10.1152/jn.1998.80.6.3383
Galovic, M., Döhler, N., Erdélyi-Canavese, B., Felbecker, A., Siebel, P., Conrad, J., Evers, S., Winklehner, M., von Oertzen, T. J., Haring, H.-P., Serafini, A., Gregoraci, G., Valente, M., Janes, F., Gigli, G. L., Keezer, M. R., Duncan, J. S., Sander, J. W., Koepp, M. J., Tettenborn, B., 2018. Prediction of late seizures after ischaemic stroke with a novel prognostic model (the SeLECT score): a multivariable prediction model development and validation study. Lancet Neurol. 17, 143-152. https://doi.org/10.1016/S1474-4422(17)30404-0
Galovic, M., Ferreira-Atuesta, C., Abraira, L., Döhler, N., Sinka, L., Brigo, F., Bentes, C., Zelano, J., Koepp, M. J., 2021. Seizures and Epilepsy After Stroke: Epidemiology, Biomarkers and Management. Drugs Aging 38, 285-299. https://doi.org/10.1007/s40266-021-00837-7
Glushakova, O. Y., Glushakov, A. A., Wijesinghe, D. S., Valadka, A. B., Hayes, R. L., Glushakov, A. V., 2017. Prospective clinical biomarkers of caspase-mediated apoptosis associated with neuronal and neurovascular damage following stroke and other severe brain injuries: Implications for chronic neurodegeneration. Brain Circ. 3, 87-108. https://doi.org/10.4103/bc.bc_27_16
Gold, C., Henze, D. A., Koch, C., Buzsiki, G., 2006. On the origin of the extracellular action potential waveform: A modeling study. J. Neurophysiol. 95, 3113-3128. https://doi.org/10.1152/jn.00979.2005
Grefkes, C., Fink, G. R., 2020. Recovery from stroke: current concepts and future perspectives. Neurol. Res. Pract. 2, 17. https://doi.org/10.1186/s42466-020-00060-6
Hasan, F., Gordon, C., Wu, D., Huang, H.-C., Yuliana, L. T., Susatia, B., Marta, O. F. D., Chiu, H.-Y., 2021. Dynamic Prevalence of Sleep Disorders Following Stroke or Transient Ischemic Attack: Systematic Review and Meta-Analysis. Stroke 52, 655-663. https://doi.org/10.1161/STROKEAHA.120.029847
He, J. Q., Sussman, E. S., Steinberg, G. K., 2020. Revisiting Stem Cell-Based Clinical Trials for Ischemic Stroke. Front Aging Neurosci 12, 575990.
Hodgkin, A. L., Huxley, A. F., 1952. A quantitative description of membrane current and its application to conduction and excitation in nerve. J. Physiol. 117, 500-544.
Jiang, Yuehua, et al. Pluripotency of mesenchymal stem cells derived from adult marrow. Nature 418.6893 (2002): 41-49.
Jin, K., Wang, X., Xie, L., Mao, X. O., Greenberg, D. A., 2010. Transgenic ablation of doublecortin-expressing cells suppresses adult neurogenesis and worsens stroke outcome in mice. Proc. Natl. Acad. Sci. U.S.A 107, 7993-7998. https://doi.org/10.1073/pnas.1000154107
Kaiser, J., Maibach, M., Salpeter, I., Hagenbuch, N., de Souza, V. B. C., Robinson, M. D., Schwab, M. E., 2019. The Spinal Transcriptome after Cortical Stroke: In Search of Molecular Factors Regulating Spontaneous Recovery in the Spinal Cord. J. Neurosci. Off. J. Soc. Neurosci. 39, 4714-4726. https://doi.org/10.1523/JNEUROSCI.2571-18.2019
Katan, M., Luft, A., 2018. Global Burden of Stroke. Semin. Neurol. 38, 208-211. https://doi.org/10.1055/s-0038-1649503
Kawabori, M., Weintraub, A. H., Imai, H., Zinkevych, L., McAllister, P., Steinberg, G. K., Frishberg, B. M., Yasuhara, T., Chen, J. W., Cramer, S. C., Achrol, A. S., Schwartz, N. E., Suenaga, J., Lu, D. C., Semeniv, I., Nakamura, H., Kondziolka, D., Chida, D., Kaneko, T., Karasawa, Y., Paadre, S., Nejadnik, B., Bates, D., Stonehouse, A. H., Richardson, R. M., Okonkwo, D. O., 2021. Cell Therapy for Chronic TBI: Interim Analysis of the Randomized Controlled STEMTRA Trial. Neurology 96, e1202-1214. https://doi.org/10.1212/WNL.0000000000011450
Kawauchi, S., Yasuhara, T., Kin, K., Yabuno, S., Sugahara, C., Nagase, T., Hosomoto, K., Okazaki, Y., Tomita, Y., Umakoshi, M., Sasaki, T., Kameda, M., Borlongan, C. V., Date, I., 2022. Transplantation of modified human bone marrow-derived stromal cells affords therapeutic effects on cerebral ischemia in rats. CNS Neurosci. Ther. 28, 1974-1985. https://doi.org/10.1111/cns.13947
Koenig, J. B., Cantu, D., Low, C., Sommer, M., Noubary, F., Croker, D., . . . & Dulla, C. G. 2019. Glycolytic inhibitor 2-deoxyglucose prevents cortical hyperexcitability after traumatic brain injury. JCI insight, 4(11), e126506.
Kokaia, M., Ernfors, P., Kokaia, Z., Elmér, E., Jaenisch, R., Lindvall, O., 1995. Suppressed epileptogenesis in BDNF mutant mice. Exp. Neurol. 133, 215-224. https://doi.org/10.1006/exnr.1995.1024
Korley, F. K., Jain, S., Sun, X., Puccio, A. M., Yue, J. K., Gardner, R. C., Wang, K. K. W., Okonkwo, D. O., Yuh, E. L., Mukherjee, P., Nelson, L. D., Taylor, S. R., Markowitz, A. J., Diaz-Arrastia, R., Manley, G. T., TRACK-TBI Study Investigators, 2022. Prognostic value of day-of-injury plasma GFAP and UCH-L1 concentrations for predicting functional recovery after traumatic brain injury in patients from the US TRACK-TBI cohort: an observational cohort study. Lancet Neurol. 21, 803-813. https://doi.org/10.1016/S1474-4422(22)00256-3
Labat-gest, V., Tomasi, S., 2013. Photothrombotic Ischemia: A Minimally Invasive and Reproducible Photochemical Cortical Lesion Model for Mouse Stroke Studies. J. Vis. Exp. JoVE 50370. https://doi.org/10.3791/50370
Langfelder, P., Horvath, S., 2008. WGCNA: an R package for weighted correlation network analysis. BMC Bioinformatics 9, 559. https://doi.org/10.1186/1471-2105-9-559
Le Blon, D., Hoornaert, C., Detrez, J. R., Bevers, S., Daans, J., Goossens, H., De Vos, W. H., Berneman, Z., Ponsaerts, P., 2017. Immune remodelling of stromal cell grafts in the central nervous system: therapeutic inflammation or (harmless) side-effect?J. Tissue Eng. Regen. Med. 11, 2846-2852. https://doi.org/10.1002/term.2188
Li, S., 2017. Spasticity, Motor Recovery, and Neural Plasticity after Stroke. Front. Neurol. 8, 120. https://doi.org/10.3389/fneur.2017.00120
Lin, Y., Yao, M., Wu, H., Wu, F., Cao, S., Ni, H., Dong, J., Yang, D., Sun, Y., Kou, X., Li, J., Xiao, H., Chang, L., Wu, J., Liu, Y., Luo, C., Zhu, D., 2021. Environmental enrichment implies GAT-1 as a potential therapeutic target for stroke recovery. Theranostics 11, 3760-3780. https://doi.org/10.7150/thno.53316
Linnerbauer, M., Rothhammer, V., 2020. Protective Functions of Reactive Astrocytes Following Central Nervous System Insult. Front. Immunol. 11.
Liu, F., Cheng, X., Zhong, S., Liu, C., Jolkkonen, J., Zhang, X., Liang, Y., Liu, Z., Zhao, C., 2020b. Communications Between Peripheral and the Brain-Resident Immune System in Neuronal Regeneration After Stroke. Front. Immunol. 11, 1931. https://doi.org/10.3389/fimmu.2020.01931
Liu, H., Povysheva, N., Rose, M. E., Mi, Z., Banton, J. S., Li, W., Chen, F., Reay, D. P., Barrionuevo, G., Zhang, F., Graham, S. H., 2019. Role of UCHL1 in axonal injury and functional recovery after cerebral ischemia. Proc. Natl. Acad. Sci. U.S.A 116, 4643-4650. https://doi.org/10.1073/pnas.1821282116
Liu, W., Wang, X., O'Connor, M., Wang, G., Han, F., 2020a. Brain-Derived Neurotrophic Factor and Its Potential Therapeutic Role in Stroke Comorbidities. Neural Plast. 2020, 1969482. https://doi.org/10.1155/2020/1969482
Liu, Z., Li, Y., Zhang, Z. G., Cui, X., Cui, Y., Lu, M., Savant-Bhonsale, S., Chopp, M., 2010. Bone marrow stromal cells enhance inter- and intracortical axonal connections after ischemic stroke in adult rats. J. Cereb. Blood Flow Metab. Off. J. Int. Soc. Cereb. Blood Flow Metab. 30, 1288-1295. https://doi.org/10.1038/jcbfm.2010.8
Lohr, K. M., Miller, G. W., 2014. VMAT2 and Parkinson's disease: harnessing the dopamine vesicle. Expert Rev. Neurother. 14, 1115-1117. https://doi.org/10.1586/14737175.2014.960399
Lorente, L., Martin, M. M., Argueso, M., Ramos, L., Solé-Violán, J., Riaño-Ruiz, M., Jiménez, A., Borreguero-León, J. M., 2015. Serum caspase-3 levels and mortality are associated in patients with severe traumatic brain injury. BMC Neurol. 15, 228. https://doi.org/10.1186/s12883-015-0485-z
Lorente, L., Martín, M. M., Pérez-Cejas, A., Gonzilez-Rivero, A. F., Sabatel, R., Ramos, L., Argueso, M., Solé-Violán, J., Cáceres, J. J., Jiménez, A., Garcia-Marín, V., 2020. Non-Survivor Ischemic Stroke Patients Maintain High Serum Caspase-Cleaved Cytokeratin-18 Levels. Brain Sci. 10, 132. https://doi.org/10.3390/brainsci10030132
Lorente, L., Martín, M. M., Pérez-Cejas, A., González-Rivero, A. F., Sabatel, R., Ramos, L., Argueso, M., Solé-Violán, J., Cáceres, J. J., Jiménez, A., García-Marín, V., 2019. Serum Caspase-3 Levels and Early Mortality of Patients with Malignant Middle Cerebral Artery Infarction. Neurocrit. Care 31, 486-493. https://doi.org/10.1007/s12028-019-00739-y
Luo, H., Zhang, Y., Zhang, Z., Jin, Y., 2012. The protection of MSCs from apoptosis in nerve regeneration by TGFβ1 through reducing inflammation and promoting VEGF-dependent angiogenesis. Biomaterials 33, 4277-4287. https://doi.org/10.1016/j.biomaterials.2012.02.042
Lv, Q., Zhang, J., Pan, Y., Liu, X., Miao, L., Peng, J., Song, L., Zou, Y., Chen, X., 2022. Somatosensory Deficits After Stroke: Insights From MRI Studies. Front. Neurol. 13, 891283. https://doi.org/10.3389/fneur.2022.891283
Ma, L., Wu, S., Gusdon, A. M., Chen, H., Hu, H., Paz, A. S., Aronowski, J., Savarraj, J. P., Kitagawa, R. S., Choi, H. A., Ren, X. S., 2022. Cathepsin L and acute ischemic stroke: A mini-review. Front. Stroke 1. https://doi.org/10.3389/fstro.2022.1050536
Maeda, S., Miyagawa, S., Kawamura, T., Shibuya, T., Watanabe, K., Nakagawa, T., Harada, A., Chida, D., Sawa, Y., 2021. Notch signaling-modified mesenchymal stem cells improve tissue perfusion by induction of arteriogenesis in a rat hindlimb ischemia model. Sci. Rep. 11, 2543. https://doi.org/10.1038/s41598-021-82284-3
Markgraf, C. G., Kraydieh, S., Prado, R., Watson, B. D., Dietrich, W. D., & Ginsberg, M. D. (1993). Comparative histopathologic consequences of photothrombotic occlusion of the distal middle cerebral artery in Sprague-Dawley and Wistar rats. Stroke, 24(2), 286-292.
Medalla, M., Chang, W., Calderazzo, S. M., Go, V., Tsolias, A., Goodliffe, J. W., Pathak, D., De Alba, D., Pessina, M., Rosene, D. L., Buller, B., Moore, T. L., 2020. Treatment with Mesenchymal-Derived Extracellular Vesicles Reduces Injury-Related Pathology in Pyramidal Neurons of Monkey Perilesional Ventral Premotor Cortex. J. Neurosci. 40, 3385-3407. https://doi.org/10.1523/JNEUROSCI.2226-19.2020
Meis, S., Endres, T., Munsch, T., Lessmann, V., 2019. Impact of Chronic BDNF Depletion on GABAergic Synaptic Transmission in the Lateral Amygdala. Int. J. Mol. Sci. 20, 4310. https://doi.org/10.3390/ijms20174310
Menon, P., Higashihara, M., van den Bos, M., Geevasinga, N., Kiernan, M. C., & Vucic, S. 2020. Cortical hyperexcitability evolves with disease progression in ALS. Annals of clinical and translational neurology, 7(5), 733-741.
Mikulec, A. A., Pittson, S., Amagasu, S. M., Monroe, F. A., MacIver, M. B., 1998. Halothane depresses action potential conduction in hippocampal axons. Brain Res. 796, 231-238. https://doi.org/10.1016/s0006-8993(98)00348-5
Mishra, N. K., Engel, J., Liebeskind, D. S., Sharma, V. K., Hirsch, L. J., Kasner, S. E., French, J. A., Devinsky, O., Friedman, A., Dawson, J., Quinn, T. J., Selim, M., de Havenon, A., Yasuda, C. L., Cendes, F., Benninger, F., Zaveri, H. P., Burneo, J. G., Srivastava, P., Bhushan Singh, M., Bhatia, R., Vishnu, V. Y., Bentes, C., Ferro, J., Weiss, S., Sivaraju, A., Kim, J. A., Galovic, M., Gilmore, E. J., Pitkanen, A., Davis, K., Sansing, L. H., Sheth, K. N., Paz, J. T., Singh, A., Sheth, S., Worrall, B. B., Grotta, J. C., Casillas-Espinos, P. M., Chen, Z., Nicolo, J.-P., Yan, B., Kwan, P., for International Post Stroke Epilepsy Research Consortium (IPSERC), 2022. International Post Stroke Epilepsy Research Consortium (IPSERC): A consortium to accelerate discoveries in preventing epileptogenesis after stroke. Epilepsy Behav. EB 127, 108502. https://doi.org/10.1016/j.yebeh.2021.108502
Mitzdorf, U., 1985. Current source-density method and application in cat cerebral cortex: investigation of evoked potentials and EEG phenomena. Physiol. Rev. 65, 37-100. https://doi.org/10.1152/physrev.1985.65.1.37
Mumm, Jeffrey S., and Raphael Kopan. Notch signaling: from the outside in. Developmental biology 228.2 (2000): 151-165.
Nagai, J., Yu, X., Papouin, T., Cheong, E., Freeman, M. R., Monk, K. R., Hastings, M. H., Haydon, P. G., Rowitch, D., Shaham, S., Khakh, B. S., 2021. Behaviorally consequential astrocytic regulation of neural circuits. Neuron 109, 576-596. https://doi.org/10.1016/j.neuron.2020.12.008
Ng, K. S., Kuncewicz, T. M., Karp, J. M., 2015. Beyond Hit-and-Run: Stem Cells Leave a Lasting Memory. Cell Metab. 22, 541-543. https://doi.org/10.1016/j.cmet.2015.09.019
Nugent, F. S., Hwong, A. R., Udaka, Y., Kauer, J. A., 2008. High-frequency afferent stimulation induces long-term potentiation of field potentials in the ventral tegmental area. Neuropsychopharmacol. Off. Publ. Am. Coll. Neuropsychopharmacol. 33, 1704-1712. https://doi.org/10.1038/sj.npp.1301561
Oldham, M. C., Langfelder, P., Horvath, S., 2012. Network methods for describing sample relationships in genomic datasets: application to Huntington's disease. BMC Syst. Biol. 6, 63. https://doi.org/10.1186/1752-0509-6-63
Olsen, T. S., Høgenhaven, H., Thage, O., 1987. Epilepsy after stroke. Neurology 37, 1209-1211. https://doi.org/10.1212/wnl.37.7.1209
Pai, E. L.-L., Vogt, D., Clemente-Perez, A., McKinsey, G. L., Oho, F. S., Hu, J. S., Wimer, M., Paul, A., Darbandi, S. F., Pla, R., Nowakowski, T. J., Goodrich, L. V., Paz, J. T., Rubenstein, J. L. R., 2019. Mafb and c-Maf Have Prenatal Compensatory and Postnatal Antagonistic Roles in Cortical Interneuron Fate and Function. Cell Rep. 26, 1157-1173.e5. https://doi.org/10.1016/j.celrep.2019.01.031
Paolucci, S., Iosa, M., Toni, D., Barbanti, P., Bovi, P., Cavallini, A., Candeloro, E., Mancini, A., Mancuso, M., Monaco, S., Pieroni, A., Recchia, S., Sessa, M., Strambo, D., Tinazzi, M., Cruccu, G., Truini, A., Neuropathic pain special interest group of the Italian Neurological Society, 2016. Prevalence and Time Course of Post-Stroke Pain: A Multicenter Prospective Hospital-Based Study. Pain Med. Malden Mass 17, 924-930. https://doi.org/10.1093/pm/pnv019
Paz, J. T., Christian, C. A., Parada, I., Prince, D. A., Huguenard, J. R., 2010. Focal cortical infarcts alter intrinsic excitability and synaptic excitation in the reticular thalamic nucleus. J. Neurosci. Off. J. Soc. Neurosci. 30, 5465-5479. https://doi.org/10.1523/JNEUROSCI.5083-09.2010
Paz, J. T., Davidson, T. J., Frechette, E. S., Delord, B., Parada, I., Peng, K., Deisseroth, K., Huguenard, J. R., 2013. Closed-loop optogenetic control of thalamus as a tool for interrupting seizures after cortical injury. Nat Neurosci 16, 64-70. https://doi.org/10.1038/nn.3269
Paz, J. T., Huguenard, J. R., 2015. Microcircuits and their interactions in epilepsy: is the focus out of focus?Nat. Neurosci. 18, 351-359. https://doi.org/10.1038/nn.3950
Pendlebury, S. T., Poole, D., Burgess, A., Duerden, J., Rothwell, P. M., Oxford Vascular Study, 2020. APOE-ε4 Genotype and Dementia Before and After Transient Ischemic Attack and Stroke: Population-Based Cohort Study. Stroke 51, 751-758. https://doi.org/10.1161/STROKEAHA.119.026927
Pettersen, K. H., Devor, A., Ulbert, I., Dale, A. M., Einevoll, G. T., 2006. Current-source density estimation based on inversion of electrostatic forward solution: effects of finite extent of neuronal activity and conductivity discontinuities. J. Neurosci. Methods 154, 116-133. https://doi.org/10.1016/j.jneumeth.2005.12.005
Pu, L., Wang, L., Zhang, R., Zhao, T., Jiang, Y., Han, L., 2023. Projected Global Trends in Ischemic Stroke Incidence, Deaths and Disability-Adjusted Life Years From 2020 to 2030. Stroke 54, 1330-1339. https://doi.org/10.1161/STROKEAHA.122.040073
Qiao, S.-Y., Shang, K., Chu, Y.-H., Yu, H.-H., Chen, X., Qin, C., Pan, D.-J., Tian, D.-S., 2022. Apolipoprotein E ε4 Polymorphism as a Risk Factor for Ischemic Stroke: A Systematic Review and Meta-Analysis. Dis. Markers 2022, 1407183. https://doi.org/10.1155/2022/1407183
Quattromani, M. J., Pruvost, M., Guerreiro, C., Backlund, F., Englund, E., Aspberg, A., Jaworski, T., Hakon, J., Ruscher, K., Kaczmarek, L., Vivien, D., Wieloch, T., 2018. Extracellular Matrix Modulation Is Driven by Experience-Dependent Plasticity During Stroke Recovery. Mol. Neurobiol. 55, 2196-2213. https://doi.org/10.1007/s12035-017-0461-2
Ranganath, S. H., Levy, O., Inamdar, M. S., Karp, J. M., 2012. Harnessing the Mesenchymal Stem Cell Secretome for the Treatment of Cardiovascular Disease. Cell Stem Cell 10, 244-258. https://doi.org/10.1016/j.stem.2012.02.005
Rani, S., Ryan, A. E., Griffin, M. D., Ritter, T., 2015. Mesenchymal Stem Cell-derived Extracellular Vesicles: Toward Cell-free Therapeutic Applications. Mol. Ther. 23, 812-823. https://doi.org/10.1038/mt.2015.44
Raudvere, U., Kolberg, L., Kuzmin, I., Arak, T., Adler, P., Peterson, H., Vilo, J., 2019. g:Profiler: a web server for functional enrichment analysis and conversions of gene lists (2019 update). Nucleic Acids Res. 47, W191-W198. https://doi.org/10.1093/nar/gkz369
Ren, C., Kobeissy, F., Alawieh, A., Li, Na, Li, Ning, Zibara, K., Zoltewicz, S., Guingab-Cagmat, J., Lamer, S. F., Ding, Y., Hayes, R. L., Ji, X., Mondello, S., 2016. Assessment of Serum UCH-L1 and GFAP in Acute Stroke Patients. Sci. Rep. 6, 24588. https://doi.org/10.1038/srep24588
Rilstone, J. J., Alkhater, R. A., Minassian, B. A., 2013. Brain dopamine-serotonin vesicular transport disease and its treatment. N. Engl. J. Med. 368, 543-550. https://doi.org/10.1056/NEJMoa1207281
Ritter-Makinson, S., Clemente-Perez, A., Higashikubo, B., Cho, F. S., Holden, S. S., Bennett, E., Chkhaidze, A., Eelkman Rooda, O. H. J., Comet, M.-C., Hoebeek, F. E., Yamakawa, K., Cilio, M. R., Delord, B., Paz, J. T., 2019. Augmented Reticular Thalamic Bursting and Seizures in Scnla-Dravet Syndrome. Cell Rep. 26, 54-64.e6. https://doi.org/10.1016/j.celrep.2018.12.018
Schroeter, Eric H., Jeffrey A. Kisslinger, and Raphael Kopan. Notch-1 signalling requires ligand-induced proteolytic release of intracellular domain. Nature 393.6683 (1998): 382-386.
Sofroniew, M. V., 2020. Astrocyte Reactivity: Subtypes, States, and Functions in CNS Innate Immunity. Trends Immunol. 41, 758-770. https://doi.org/10.1016/j.it.2020.07.004 Steinberg, G. K., Kondziolka, D., Wechsler, L. R., Lunsford, L. D., Kim, A. S., Johnson, J. N.,
Bates, D., Poggio, G., Case, C., McGrogan, M., Yankee, E. W., Schwartz, N. E., 2018. Two-year safety and clinical outcomes in chronic ischemic stroke patients after implantation of modified bone marrow-derived mesenchymal stem cells (SB623): a phase 1/2a study. J. Neurosurg. 1-11. https://doi.org/10.3171/2018.5.JNS173147
Steinschneider, M., Tenke, C. E., Schroeder, C. E., Javitt, D. C., Simpson, G. V., Arezzo, J. C., Vaughan, H. G., 1992. Cellular generators of the cortical auditory evoked potential initial component. Electroencephalogr. Clin. Neurophysiol. 84, 196-200. https://doi.org/10.1016/0168-5597(92)90026-8
Sun, D.-B., Xu, M.-J., Chen, Q.-M., Hu, H.-T., 2017. Significant elevation of serum caspase-3 levels in patients with intracerebral hemorrhage. Clin. Chim. Acta Int. J. Clin. Chem. 471, 62-67. https://doi.org/10.1016/j.cca.2017.05.021
Tajiri, N., Kaneko, Y., Shinozuka, K., Ishikawa, H., Yankee, E., McGrogan, M., Case, C., Borlongan, C. V., 2013. Stem cell recruitment of newly formed host cells via a successful seduction?Filling the gap between neurogenic niche and injured brain site. PloS One 8, e74857. https://doi.org/10.1371/journal.pone.0074857
Tanaka, T., Saito, H., Matsuki, N., 1997. Inhibition of GABAA Synaptic Responses by Brain-Derived Neurotrophic Factor (BDNF) in Rat Hippocampus. J. Neurosci. 17, 2959-2966. https://doi.org/10.1523/JNEUROSCI.17-09-02959.1997
Tanigaki, K., Sundgren, N., Khera, A., Vongpatanasin, W., Mineo, C., Shaul, P. W., 2015. Fc Receptors and Ligands and Cardiovascular Disease. Circ. Res. 116, 368-384. https://doi.org/10.1161/CIRCRESAHA.116.302795
Tate, C. C., Fonck, C., McGrogan, M., Case, C. C., 2010. Human mesenchymal stromal cells and their derivative, SB623 cells, rescue neural cells via trophic support following in vitro ischemia. Cell Transplant. 19, 973-984. https://doi.org/10.3727/096368910X494885
Toma, C., Wagner, W. R., Bowry, S., Schwartz, A., Villanueva, F., 2009. Fate of culture-expanded mesenchymal stem cells in the microvasculature: in vivo observations of cell kinetics. Circ. Res. 104, 398-402. https://doi.org/10.1161/CIRCRESAHA.108.187724
Uzdensky, A. B., 2018. Photothrombotic Stroke as a Model of Ischemic Stroke. Transl. Stroke Res. 9, 437-451. https://doi.org/10.1007/s12975-017-0593-8
Vaz, S. H., Jørgensen, T. N., Cristóvão-Ferreira, S., Duflot, S., Ribeiro, J. A., Gether, U., Sebastião, A. M., 2011. Brain-derived neurotrophic factor (BDNF) enhances GABA transport by modulating the trafficking of GABA transporter-1 (GAT-1) from the plasma membrane of rat cortical astrocytes. J. Biol. Chem. 286, 40464-40476. https://doi.org/10.1074/jbc.M111.232009
Volkman, R., Offen, D., 2017. Concise Review: Mesenchymal Stem Cells in Neurodegenerative Diseases. Stem Cells 35, 1867-1880.
Volz, L. J., Vollmer, M., Michely, J., Fink, G. R., Rothwell, J. C., Grefkes, C., 2017. Time-dependent functional role of the contralesional motor cortex after stroke. Neurolmage Clin. 16, 165-174. https://doi.org/10.1016/j.nicl.2017.07.024
Wang, F., Zhang, C., Hou, S., Geng, X., 2019. Synergistic Effects of Mesenchymal Stem Cell Transplantation and Repetitive Transcranial Magnetic Stimulation on Promoting Autophagy and Synaptic Plasticity in Vascular Dementia. J. Gerontol. A. Biol. Sci. Med. Sci. 74, 1341-1350. https://doi.org/10.1093/gerona/gly221
Wang, S., Hesen, R., Mossink, B., Kasri, N. N. and Schubert, D., 2023. Generation of glutamatergic/GABAergic neuronal co-cultures derived from human induced pluripotent stem cells for characterizing E/I balance in vitro. STAR protocols, 4(1), p.101967.
Weinmaster, Gerry, Veronica J. Roberts, and Greg Lemke. A homolog of Drosophila Notch expressed during mammalian development. Development 113.1 (1991): 199-205.
Wu, B.-S., Chen, S.-F., Huang, S.-Y., Ou, Y.-N., Deng, Y.-T., Chen, S.-D., Dong, Q., Yu, J.-T., 2022. Identifying causal genes for stroke via integrating the proteome and transcriptome from brain and blood. J. Transl. Med. 20, 181. https://doi.org/10.1186/s12967-022-03377-9
Xin, H., Li, Y., Cui, Y., Yang, J. J., Zhang, Z. G., Chopp, M., 2013. Systemic administration of exosomes released from mesenchymal stromal cells promote functional recovery and neurovascular plasticity after stroke in rats. J. Cereb. Blood Flow Metab. Off. J. Int. Soc. Cereb. Blood Flow Metab. 33, 1711-1715. https://doi.org/10.1038/jcbfm.2013.152
Xiong, X.-Y., Tang, Y., Yang, Q.-W., 2022. Metabolic changes favor the activity and heterogeneity of reactive astrocytes. Trends Endocrinol. Metab. TEM 33, 390-400. https://doi.org/10.1016/j.tem.2022.03.001
Xu, M. Y., 2019. Poststroke seizure: optimising its management. Stroke Vasc. Neurol. 4, 48-56. https://doi.org/10.1136/svn-2018-000175
Yabuno, S., Yasuhara, T., Nagase, T., Kawauchi, S., Sugahara, C., Okazaki, Y., Hosomoto, K., Sasada, S., Sasaki, T., Tajiri, N., Borlongan, C. V., Date, I., 2023. Synergistic therapeutic effects of intracerebral transplantation of human modified bone marrow-derived stromal cells (SB623) and voluntary exercise with running wheel in a rat model of ischemic stroke. Stem Cell Res. Ther. 14, 10. https://doi.org/10.1186/s13287-023-03236-4
Yang, X., Yan, S., Wang, P., Wang, G., 2022. Identification of Hub Genes in the Pathogenesis of Ischemic Stroke Based on Bioinformatics Analysis. J. Korean Neurosurg. Soc. 65, 697-709. https://doi.org/10.3340/jkns.2021.0200
Yarmohammadi, F., Hayes, A. W., Karimi, G., 2022. Sorting nexins as a promising therapeutic target for cardiovascular disorders: An updated overview. Exp. Cell Res. 419, 113304. https://doi.org/10.1016/j.yexcr.2022.113304
Yasuhara, T., Matsukawa, N., Hara, K., Maki, M., Ali, M. M., Yu, S. J., Bae, E., Yu, G., Xu, L., McGrogan, M., Bankiewicz, K., Case, C., Borlongan, C. V., 2009. Notch-induced rat and human bone marrow stromal cell grafts reduce ischemic cell loss and ameliorate behavioral deficits in chronic stroke animals. Stem Cells Dev. 18, 1501-1514. https://doi.org/10.1089/scd.2009.0011
Yigit, I., Atescelik, M., Yilmaz, M., Goktekin, M. C., Gurger, M., Ilhan, N., 2017. Investigation of UCH-L1 levels in ischemic stroke, intracranial hemorrhage and metabolic disorder induced impaired consciousness. Am. J. Emerg. Med. 35, 1895-1898. https://doi.org/10.1016/j.ajem.2017.06.032
Zhaolong Zhang, Lv, M., Zhou, X., Cui, Y., 2022. Roles of peripheral immune cells in the recovery of neurological function after ischemic stroke. Front. Cell. Neurosci. 16, 1013905. https://doi.org/10.3389/fncel.2022.1013905
Zheng, P.-F., Chen, L.-Z., Liu, P., Pan, H. W., Fan, W.-J., Liu, Z.-Y., 2022. Identification of immune-related key genes in the peripheral blood of ischaemic stroke patients using a weighted gene co-expression network analysis and machine learning. J. Transl. Med. 20, 361. https://doi.org/10.1186/s12967-022-03562-w
Zhou, Q., Dong, Y., Wang, K., Wang, Z., Ma, B., Yang, B., 2023. A comprehensive analysis of the hub genes for oxidative stress in ischemic stroke. Front. Neurosci. 17, 1166010. https://doi.org/10.3389/fnins.2023.1166010

We claim:

1. A method of treating chronic cortical hyperexcitability, the method comprising:
administering vandefitemcel to a region of the brain of a subject to reduce chronic cortical hyperexcitability in the subject, wherein the vandefitemcel are cells descended from mesenchymal stem cells (MSCs) transiently-transfected by a polynucleotide encoding a Notch intracellular domain (NICD), wherein the vandefitemcel treats the chronic cortical hyperexcitability by at least one of:
increasing production of gamma-aminobutyric acid (GABA) transporters 1 (GAT1) in the brain of the subject; and
increasing production of brain-derived neurotrophic factors (BDNF) in the brain of the subject.

2. The method of claim 1, wherein the vandefitemcel treats the chronic cortical hyperexcitability by increasing the production of GAT1 in a peri-stroke cortex of the brain of the subject.

3. The method of claim 1, wherein the vandefitemcel treats the chronic cortical hyperexcitability by increasing the production of BDNF in a peri-stroke cortex of the brain of the subject.

4. The method of claim 1, wherein the vandefitemcel treats the chronic cortical hyperexcitability by increasing the production of BDNF in a corpus callosum of the brain of the subject.

5. The method of claim 1, wherein the vandefitemcel treats the chronic cortical hyperexcitability by increasing the production of BDNF in a somatosensory thalamus of the brain of the subject.

6. The method of claim 1, wherein the vandefitemcel treats the chronic cortical hyperexcitability by increasing the production of BDNF in an internal capsule of the brain of the subject.

7. The method of claim 1, wherein the vandefitemcel treats the chronic cortical hyperexcitability by increasing the production of BDNF in a contralesional hemisphere of the brain of the subject.

8. The method of claim 1, wherein the vandefitemcel treats the chronic cortical hyperexcitability by increasing the production of BDNF in an ipsilesional hemisphere of the brain of the subject.

9. The method of claim 1, wherein the vandefitemcel treats the chronic cortical hyperexcitability by increasing production of doublecortin-positive (DCX⁺) neuronal progenitor cells (NPCs) in the brain of the subject.

10. The method of claim 1, wherein the vandefitemcel treats the chronic cortical hyperexcitability by increasing production of oligodendrocyte precursor cells (OPCs).

11. The method of claim 10, wherein the OPCs are oligodendrocyte transcription factor 2-positive (Olig2⁺) OPCs.

12. The method of claim 10, wherein the OPCs are proliferating cell nuclear antigen-positive (PCNA⁺) OPCs.

13. The method of claim 1, wherein the vandefitemcel treats the chronic cortical hyperexcitability by increasing production of myelin basic protein (MBP) in a contralesional cortex of the subject.

14. The method of claim 1, wherein the vandefitemcel treats the chronic cortical hyperexcitability by increasing production of glial fibrillary acidic protein-positive (GFAP⁺) astrocytes in the brain of the subject.

15. The method of claim 1, wherein the vandefitemcel treats the chronic cortical hyperexcitability by increasing production of ionized calcium-binding adaptor molecule 1-positive (Iba1⁺) microglia in the brain of the subject.

16. The method of claim 1, wherein the vandefitemcel treats the chronic cortical hyperexcitability by inducing synaptogenesis in a peri-stroke cortex of the brain of the subject.

17. The method of claim 1, wherein the vandefitemcel treats the chronic cortical hyperexcitability by reducing glutamatergic synaptic vesicles in an ipsilesional region of a peri-stroke cortex of the brain compared to a contralesional region of the peri-stroke cortex.

18. The method of claim 1, wherein the vandefitemcel treats the chronic cortical hyperexcitability by reducing perineuronal nets in an ipsilesional region of a peri-stroke cortex of the brain compared to a contralesional region of the peri-stroke cortex.

19. The method of claim 1, wherein administering the vandefitemcel further comprises administering the vandefitemcel by intracerebral implantation.

20. The method of claim 19, wherein administering the vandefitemcel further comprises injecting the vandefitemcel at multiple sites within the region of the brain.

21. The method of claim 19, wherein administering the vandefitemcel further comprises administering the vandefitemcel stereotactically via a burr hole in a skull of the subject.

22. The method of claim 1, wherein the region of the brain is a peri-stroke cortex.

23. The method of claim 1, wherein administering the vandefitemcel further comprises administering the vandefitemcel by parenteral administration.

24. The method of claim 1, wherein the vandefitemcel is suspended in a sterile isotonic crystalloid solution.

25. The method of claim 1, wherein administering the vandefitemcel further comprises administering between about 1.0 million cells and 10.0 million cells.

26. The method of claim 1, wherein the vandefitemcel is made by a method comprising:
providing a culture of MSCs;
contacting the culture of MSCs with the polynucleotide encoding the NICD, wherein the polynucleotide does not encode a full-length Notch protein,
selecting cells that comprise the polynucleotide; and
further culturing the selected cells in the absence of selection for the polynucleotide.

27. The method of claim 1, wherein the MSCs are human bone marrow-derived cells.

28. A method of reversing stroke-induced changes in whole-blood gene expression, the method comprising:
administering vandefitemcel to a region of the brain of a subject, wherein the vandefitemcel are cells descended from mesenchymal stem cells (MSCs) transiently-transfected by a polynucleotide encoding a Notch intracellular domain (NICD), wherein the vandefitemcel reverses stroke-induced changes in whole-blood gene expression by at least one of:
downregulating CD8a gene expression;
downregulating CD8b gene expression;
downregulating Uchl1 gene expression;
downregulating Casp3 gene expression;
downregulating Ube2s gene expression;
downregulating Slc18a2 gene expression; and
upregulating Fcgr2b gene expression.

29. The method of claim 28, wherein the vandefitemcel reverses stroke-induced changes in whole-blood gene expression by downregulating Scarf1 gene expression.

30. The method of claim 28, wherein the vandefitemcel reverses stroke-induced changes in whole-blood gene expression by downregulating Dusp1 gene expression.

31. The method of claim 28, wherein the vandefitemcel reverses stroke-induced changes in whole-blood gene expression by downregulating Csnk2a1 gene expression.

32. The method of claim 28, wherein administering the vandefitemcel further comprises administering the vandefitemcel by intracerebral implantation.

33. The method of claim 32, wherein administering the vandefitemcel further comprises injecting the vandefitemcel at multiple sites within the region of the brain.

34. The method of claim 32, wherein administering the vandefitemcel further comprises administering the vandefitemcel stereotactically via a burr hole in a skull of the subject.

35. The method of claim 28, wherein the region of the brain is a peri-stroke cortex.

36. The method of claim 28, wherein administering the vandefitemcel further comprises administering between about 1.0 million cells and 10.0 million cells.

37. The method of claim 28, wherein the vandefitemcel is made by a method comprising:
providing a culture of MSCs;
contacting the culture of MSCs with the polynucleotide encoding the NICD, wherein the polynucleotide does not encode a full-length Notch protein,
selecting cells that comprise the polynucleotide; and
further culturing the selected cells in the absence of selection for the polynucleotide.

38. The method of claim 28, wherein the MSCs are human bone marrow-derived cells.

39. The method of claim 28, wherein the vandefitemcel is suspended in a sterile isotonic crystalloid solution.

40. The method of claim 28, wherein the changes are induced by an ischemic stroke and wherein the vandefitemcel are administered thirty days or more after the ischemic stroke.

41. A method of reversing stroke-induced changes in serum protein expression, the method comprising:
administering vandefitemcel to a region of the brain of a subject, wherein the vandefitemcel are cells descended from mesenchymal stem cells (MSCs) transiently-transfected by a polynucleotide encoding a Notch intracellular domain (NICD), wherein the vandefitemcel reverses stroke-induced changes in serum protein expression by at least one of:
upregulating Filamin A protein expression;
downregulating Cathepsin L protein expression;
downregulating ApoE protein expression;
downregulating ARHGAP1 protein expression; and
downregulating TALDO1 protein expression.

42. The method of claim 41, wherein administering the vandefitemcel further comprises administering the vandefitemcel by intracerebral implantation.

43. The method of claim 42, wherein administering the vandefitemcel further comprises injecting the vandefitemcel at multiple sites within the region of the brain.

44. The method of claim 42, wherein administering the vandefitemcel further comprises administering the vandefitemcel stereotactically via a burr hole in a skull of the subject.

45. The method of claim 41, wherein the region of the brain is a peri-stroke cortex.

46. The method of claim 41, wherein administering the vandefitemcel further comprises administering between about 1.0 million cells and 10.0 million cells.

47. The method of claim 41, wherein the vandefitemcel is made by a method comprising:
providing a culture of MSCs;
contacting the culture of MSCs with the polynucleotide encoding the NICD, wherein the polynucleotide does not encode a full-length Notch protein,
selecting cells that comprise the polynucleotide; and
further culturing the selected cells in the absence of selection for the polynucleotide.

48. The method of claim 41, wherein the MSCs are human bone marrow-derived cells.

49. The method of claim 41, wherein the vandefitemcel is suspended in a sterile isotonic crystalloid solution.

50. The method of claim 41, wherein the changes are induced by an ischemic stroke and wherein the vandefitemcel are administered thirty days or more after the ischemic stroke.

51. A method of assessing an efficacy of a treatment for a chronic condition caused by stroke, the method comprising:
obtaining a first sample of blood from a subject;
administering vandefitemcel to a region of the brain of a subject, wherein the vandefitemcel are cells descended from mesenchymal stem cells (MSCs) transiently-transfected by a polynucleotide encoding a Notch intracellular domain (NICD);
obtaining a second sample of blood from a subject at least three days after administering the vandefitemcel;
comparing gene expression levels between the second sample of blood and the first sample of blood for at least one of the following biomarker genes: CD8a, CD8b, Uchl1, Casp3, Ube2s, and Slc18a2; and
classifying the vandefitemcel administration as effective in ameliorating the chronic condition caused by the stroke when at least one of the CD8a gene, the CD8b gene, the Uchl1 gene, the Casp3 gene, the Ube2s gene, and the Slc18a2 gene in the second sample of blood is downregulated relative to the first sample of blood.

52. The method of claim 51, further comprising:
comparing a gene expression level between the second sample of blood and the first sample of blood for the biomarker gene Fcgr2b; and
classifying the vandefitemcel administration as effective in ameliorating the chronic condition caused by the stroke when the biomarker gene Fcgr2b in the second sample of blood is upregulated relative to the first sample of blood.

53. The method of claim 51, further comprising:
comparing gene expression levels between the second sample of blood and the first sample of blood for at least one of the following biomarker genes: Scarf1, Dusp1, and Csnk2a1; and
classifying the vandefitemcel administration as effective in ameliorating the chronic condition caused by the stroke when at least one of the Scarf1 gene, the Dusp1 gene, and the Csnk2a1 gene in the second sample of blood is downregulated relative to the first sample of blood.

54. The method of claim 51, further comprising:
comparing protein expression levels between the second sample of blood and the first sample of blood for at least one of the following protein biomarkers: Cathepsin L, ApoE, ARHGAP1, and TALDO1;
classifying the vandefitemcel administration as effective in ameliorating the chronic condition caused by the stroke when at least one of the Cathepsin L protein biomarker, the ApoE protein biomarker, the ARHGAP1 protein biomarker, and the TALDO1 protein biomarker in the second sample of blood is downregulated relative to the first sample of blood.

55. The method of claim 51, further comprising:
comparing a protein expression level between the second sample of blood and the first sample of blood for the protein biomarker Filamin A; and
classifying the vandefitemcel administration as effective in ameliorating the chronic condition caused by the stroke when the protein biomarker Filamin A in the second sample of blood is upregulated relative to the first sample of blood.

56. The method of claim 51, wherein administering the vandefitemcel further comprises administering the vandefitemcel by intracerebral implantation.

57. The method of claim 56, wherein administering the vandefitemcel further comprises injecting the vandefitemcel at multiple sites within the region of the brain.

58. The method of claim 56, wherein administering the vandefitemcel further comprises administering the vandefitemcel stereotactically via a burr hole in a skull of the subject.

59. The method of claim 51, wherein the region of the brain is a peri-stroke cortex.

60. The method of claim 51, wherein administering the vandefitemcel further comprises administering between about 1.0 million cells and 10.0 million cells.

61. The method of claim 51, wherein the vandefitemcel is made by a method comprising:
providing a culture of MSCs;
contacting the culture of MSCs with the polynucleotide encoding the NICD, wherein the polynucleotide does not encode a full-length Notch protein,
selecting cells that comprise the polynucleotide; and
further culturing the selected cells in the absence of selection for the polynucleotide.

62. The method of claim 51, wherein the MSCs are human bone marrow-derived cells.

63. The method of claim 51, wherein the vandefitemcel is suspended in a sterile isotonic crystalloid solution.

64. The method of claim 51, wherein the stroke is ischemic stroke and wherein the vandefitemcel is administered thirty days or more after the ischemic stroke.

65. A composition for treating chronic cortical hyperexcitability, the composition comprising:
vandefitemcel in an amount between about 1.0 million cells and 10.0 million cells, wherein the vandefitemcel is produced by modifying mesenchymal stem cells derived from bone marrow; and
one or more pharmaceutically acceptable excipients,
wherein the composition treats the chronic cortical hyperexcitability by at least one of:
increasing production of gamma-aminobutyric acid (GABA) transporters 1 (GAT1) in the brain of a subject; and
increasing production of brain-derived neurotrophic factors (BDNF) in the brain of the subject.

66. The composition of claim 65, wherein the composition treats the chronic cortical hyperexcitability by increasing the production of GAT1 in a peri-stroke cortex of the brain of the subject.

67. The composition of claim 65, wherein the composition treats the chronic cortical hyperexcitability by increasing the production of BDNF in at least one of a peri-stroke cortex, corpus callosum, a somatosensory thalamus, an internal capsule, a contralesional hemisphere, and an ipsilesional hemisphere of the brain of the subject.

68. The composition of claim 65, wherein the composition treats the chronic cortical hyperexcitability by increasing production of doublecortin-positive (DCX⁺) neuronal progenitor cells (NPCs) in the brain of the subject.

69. The composition of claim 65, wherein the composition treats the chronic cortical hyperexcitability by increasing production of oligodendrocyte precursor cells (OPCs).

70. The composition of claim 69, wherein the OPCs are oligodendrocyte transcription factor 2-positive (Olig2+) OPCs.

71. The composition of claim 69, wherein the OPCs are proliferating cell nuclear antigen-positive (PCNA+) OPCs.

72. The composition of claim 65, wherein the composition treats the chronic cortical hyperexcitability by increasing production of myelin basic protein (MBP) in a contralesional cortex of the subject.

73. The composition of claim 65, wherein the composition treats the chronic cortical hyperexcitability by increasing production of glial fibrillary acidic protein-positive (GFAP⁺) astrocytes in the brain of the subject.

74. The composition of claim 65, wherein the composition treats the chronic cortical hyperexcitability by increasing production of ionized calcium-binding adaptor molecule 1-positive (Iba1⁺) microglia in the brain of the subject.

75. The composition of claim 65, wherein the composition treats the chronic cortical hyperexcitability by inducing synaptogenesis in a peri-stroke cortex of the brain of the subject.

76. The composition of claim 65, wherein the composition treats the chronic cortical hyperexcitability by reducing glutamatergic synaptic vesicles in an ipsilesional region of a peri-stroke cortex of the brain compared to a contralesional region of the peri-stroke cortex.

77. The composition of claim 65, wherein the composition treats the chronic cortical hyperexcitability by reducing perineuronal nets in an ipsilesional region of a peri-stroke cortex of the brain compared to a contralesional region of the peri-stroke cortex.

78. The composition of claim 65, wherein the vandefitemcel is made by a process comprising:
providing a culture of mesenchymal stem cells;
contacting the culture of mesenchymal stem cells with a polynucleotide encoding a Notch intracellular domain (NICD), wherein the polynucleotide does not encode a full-length Notch protein,
selecting cells that comprise the polynucleotide; and
further culturing the selected cells in the absence of selection for the polynucleotide.

79. The composition of claim 78, wherein the mesenchymal stem cells are human bone marrow-derived cells.

80. The composition of claim 65, wherein the mesenchymal stem cells are transiently-transfected with a plasmid vector comprising the polynucleotide encoding the NICD.

81. The composition of claim 65, wherein the one or more pharmaceutically acceptable excipients comprises at least one of buffers, proteins, stabilizers, and preservatives.

82. The composition of claim 65, wherein the vandefitemcel is suspended in a sterile isotonic crystalloid solution.

83. The composition of claim 65, wherein the one or more pharmaceutically acceptable excipients comprises carriers or diluents.

84. A composition for reversing stroke-induced changes in whole-blood gene expression, the composition comprising:
vandefitemcel in an amount between about 1.0 million cells and 10.0 million cells, wherein the vandefitemcel is produced by modifying mesenchymal stem cells derived from bone marrow; and
one or more pharmaceutically acceptable excipients,
wherein the composition reverses the stroke-induced changes in whole-blood gene expression by at least one of:
downregulating CD8a gene expression;
downregulating CD8b gene expression;
downregulating Uchl1 gene expression;
downregulating Casp3 gene expression;
downregulating Ube2s gene expression;
downregulating Slc18a2 gene expression; and
upregulating Fcgr2b gene expression.

85. The composition of claim 84, wherein the composition reverses stroke-induced changes in whole-blood gene expression by downregulating Scarf1 gene expression.

86. The composition of claim 84, wherein the composition reverses stroke-induced changes in whole-blood gene expression by downregulating Dusp1 gene expression.

87. The composition of claim 84, wherein the composition reverses stroke-induced changes in whole-blood gene expression by downregulating Csnk2a1 gene expression.

88. The composition of claim 84, wherein the vandefitemcel is made by a process comprising:
providing a culture of mesenchymal stem cells;
contacting the culture of mesenchymal stem cells with a polynucleotide encoding a Notch intracellular domain (NICD), wherein the polynucleotide does not encode a full-length Notch protein,
selecting cells that comprise the polynucleotide; and
further culturing the selected cells in the absence of selection for the polynucleotide.

89. The composition of claim 88, wherein the mesenchymal stem cells are human bone marrow-derived cells.

90. The composition of claim 84, wherein the mesenchymal stem cells are transiently-transfected with a plasmid vector comprising the polynucleotide encoding the NICD.

91. The composition of claim 84, wherein the one or more pharmaceutically acceptable excipients comprises at least one of buffers, proteins, stabilizers, and preservatives.

92. The composition of claim 84, wherein the vandefitemcel is suspended in a sterile isotonic crystalloid solution.

93. The composition of claim 84, wherein the one or more pharmaceutically acceptable excipients comprises carriers or diluents.

94. A composition for reversing stroke-induced changes in serum protein expression, the composition comprising:
vandefitemcel in an amount between about 1.0 million cells and 10.0 million cells, wherein the vandefitemcel is produced by modifying mesenchymal stem cells derived from bone marrow; and
one or more pharmaceutically acceptable excipients,
wherein the composition reverses the stroke-induced changes in serum protein expression by at least one of:
upregulating Filamin A protein expression;
downregulating Cathepsin L protein expression;
downregulating ApoE protein expression;
downregulating ARHGAP1 protein expression; and
downregulating TALDO1 protein expression.

95. The composition of claim 94, wherein the vandefitemcel is made by a process comprising:
providing a culture of mesenchymal stem cells;
contacting the culture of mesenchymal stem cells with a polynucleotide encoding a Notch intracellular domain (NICD), wherein the polynucleotide does not encode a full-length Notch protein,
selecting cells that comprise the polynucleotide; and
further culturing the selected cells in the absence of selection for the polynucleotide.

96. The composition of claim 95, wherein the mesenchymal stem cells are human bone marrow-derived cells.

97. The composition of claim 95, wherein the mesenchymal stem cells are transiently-transfected with a plasmid vector comprising the polynucleotide encoding the NICD.

98. The composition of claim 94, wherein the one or more pharmaceutically acceptable excipients comprises at least one of buffers, proteins, stabilizers, and preservatives.

99. The composition of claim 94, wherein the vandefitemcel is suspended in a sterile isotonic crystalloid solution.

100. The composition of claim 94, wherein the one or more pharmaceutically acceptable excipients comprises carriers or diluents.

101. A method of inducing endogenous production of brain-derived neurotrophic factors (BDNFs), the method comprising:
administering vandefitemcel to a region of the brain of a subject, wherein the vandefitemcel are cells descended from mesenchymal stem cells (MSCs) transiently-transfected by a polynucleotide encoding a Notch intracellular domain (NICD).

102. The method of claim 101, wherein the production of BDNF is increased in a peri-stroke cortex of the brain of the subject.

103. The method of claim 101, wherein the production of BDNF is increased in a corpus callosum of the brain of the subject.

104. The method of claim 101, wherein the production of BDNF is increased in a somatosensory thalamus of the brain of the subject.

105. The method of claim 101, wherein the production of BDNF is increased in an internal capsule of the brain of the subject.

106. The method of claim 101, wherein the production of BDNF is increased in a contralesional hemisphere of the brain of the subject.

107. The method of claim 101, wherein the production of BDNF is increased in an ipsilesional hemisphere of the brain of the subject.

108. A composition for inducing endogenous production of brain-derived neurotrophic factors (BDNFs), the composition comprising:
vandefitemcel in an amount between about 1.0 million cells and 10.0 million cells, wherein the vandefitemcel is produced by modifying mesenchymal stem cells derived from bone marrow; and
one or more pharmaceutically acceptable excipients.

109. The composition of claim 108, wherein the production of BDNF is increased in a peri-stroke cortex of the brain of a subject.

110. The composition of claim 108, wherein the production of BDNF is increased in a corpus callosum of the brain of a subject.

111. The composition of claim 108, wherein the production of BDNF is increased in a somatosensory thalamus of the brain of a subject.

112. The composition of claim 108, wherein the production of BDNF is increased in an internal capsule of the brain of a subject.

113. The composition of claim 108, wherein the production of BDNF is increased in a contralesional hemisphere of the brain of a subject.

114. The composition of claim 108, wherein the production of BDNF is increased in an ipsilesional hemisphere of the brain of a subject.

115. The composition of claim 108, wherein the vandefitemcel is made by a process comprising:
providing a culture of mesenchymal stem cells;
contacting the culture of mesenchymal stem cells with a polynucleotide encoding a Notch intracellular domain (NICD), wherein the polynucleotide does not encode a full-length Notch protein,
selecting cells that comprise the polynucleotide; and
further culturing the selected cells in the absence of selection for the polynucleotide.

116. The composition of claim 115, wherein the mesenchymal stem cells are human bone marrow-derived cells.

117. The composition of claim 115, wherein the mesenchymal stem cells are transiently-transfected with a plasmid vector comprising the polynucleotide encoding the NICD.

118. The composition of claim 108, wherein the one or more pharmaceutically acceptable excipients comprises at least one of buffers, proteins, stabilizers, and preservatives.

119. The composition of claim 108, wherein the vandefitemcel is suspended in a sterile isotonic crystalloid solution.

120. The composition of claim 108, wherein the one or more pharmaceutically acceptable excipients comprises carriers or diluents.
